(19) United States
(12) Patent Application Publication
(10) Pub. No.: US 2025/0115657 A1
(43) Pub. Date: Apr. 10, 2025

(54) Bispecific FC Fusion Proteins with sPD-1 and IL-15

(71) Applicant: AKSO BIOPHARMACEUTICAL, INC., Los Angeles, CA (US)

(72) Inventors: Amato J. Giaccia, Oxford (GB); Yu Miao, Sunnyvale, CA (US); Xin Eric Zhang, Newport Coast, CA (US)

(73) Assignee: AKSO BIOPHARMACEUTICAL, INC., Los Angeles, CA (US)

(21) Appl. No.: 18/729,436

(22) PCT Filed: May 27, 2022
(86) PCT No.: PCT/US2022/031445

§ 371 Date: Jul. 16, 2024

Related U.S. Application Data
(60) Provisional application No. 63/194,560, filed on May 28, 2021.

(51) Int. Cl. C07K 14/705; C07K 14/715

(52) U.S. Cl. CPC C07K 14/70521; C07K 14/7155; C07K 2319/30; C07K 2319/32; C07K 2319/75

(57) ABSTRACT

The present disclosure is directed to novel bispecific Fc fusion proteins with an IF-15 domain, an Interleukin-15 receptor alpha (IF-15Rα) sushi domain, an Fc domain and a soluble PD-1 (sPD-1) variant domain, polynucleotides encoding said bispecific Fc fusion proteins, methods of making and using thereof.

### I. FIELD

This invention relates to the field of biomolecules (e.g., cytokine and/or immune checkpoint receptors) induced or stimulated biological responses, more particularly to the field of IL-15 and/or PD-I modulated biological responses. Specifically, this invention relates to bispecific Fc fusion proteins comprising an IL-15 domain, an Interleukin-15 receptor alpha (IL-15Rα) sushi domain, an Fc domain and a soluble PD-1 (sPD-1) variant domain, polynucleotides encoding the bispecific Fc fusion proteins, methods of making the bispecific Fc fusion proteins, and methods of using the bispecific Fc fusion proteins, for example, in preventing and/or treating diseases (e.g., cancers, infections, etc.), modulating immune functions, promoting T cell and/or Natural Killer (NK) cell cytotoxicity, etc.

### II. BACKGROUND

The cytokine, interleukin-15 (IL-15), is a member of the four alpha-helix bundle family of lymphokines. IL-15 plays an important role in modulating the activity of both the innate and adaptive immune system, e.g., maintenance of the memory T-cell response to invading pathogens, inhibition of apoptosis, activation of dendritic cells, and induction of NK cell proliferation and cytotoxic activity (U.S. Ser. No. 10/899,821B2, hereby entirely incorporated by reference).

The IL-15 receptor consists of the IL-2/IL-15Rβ and γc subunits in association with a unique ligand-specific subunit, IL-15Rα, which is homologous to IL-2Rα. These receptor proteins contain protein-binding motifs termed “sushi domains.” In both humans and mice, these receptors and their cognate ligands are physically linked in the genome (Clinical Immunology (Fifth Edition) Principles and Practice 2019, Chapter 9: J. O'Shea, Massimo Gadina, Richard M. Siegel. Cytokines and Cytokine Receptors. John p. 127-155, hereby entirely incorporated by reference).

IL-15 binds to the IL-15Rα, forming cell-surface complexes. IL-15 specifically binds to the IL-15Rα with high affinity via the “sushi domain” in exon 2 of the extracellular domain of the receptor. After trans-endosomal recycling and migration back to the cell surface, these IL-15 complexes acquire the property to activate bystander cells expressing the IL-15R βy low-affinity receptor complex and induce IL-15-mediated signaling pathway (U.S. Pat. No. 10,265,382B2, hereby entirely incorporated by reference). IL-15 signaling is essential for normal immune system functions. It stimulates T cell proliferation and inhibits IL-2-mediated activation-induced cell death.

PD-1 (programmed cell death 1) is an important immune checkpoint receptor expressed by activated T cell and B cells. It functions to mediate immunosuppression. PD-1 is expressed on activated T cells, B cells, and NK cells. The ligands for PD-1 are programmed cell death 1 ligand 1 (PD-L1, alternatively B7-H1) and programmed cell death 1 ligand 2 (PD-L2, alternatively B7-DC) which are expressed on many tumor cells and antigen-presenting cells, such as monocytes, dendritic cells (DC) and macrophages (U.S. Pat. No. 10,588,938 B2, U.S. application Ser. No. 16/569,105, WO 2020/056085 A1, hereby entirely incorporated by reference).

PD-1 acts to deliver a negative immune response signal when induced in T cells. Activation of PD-1 via selective binding to one of its ligands activates an inhibitory immune response that decreases T cell proliferation and/or the intensity and/or duration of a T cell response. PD-1 also regulates effector T cell activity in peripheral tissues in response to infection or tumor progression (Pardoll, Nat Rev Cancer, 2012, 12(4):252-264, hereby entirely incorporated by reference).

Endogenous immune checkpoints, such as the PD-1 signaling pathway, which normally terminate immune responses to mitigate collateral tissue damage, can be co-opted by tumors to evade immune destruction. The interaction between PD-L1 and PD-1 in cancers can decrease the number of tumor-infiltrating immune cells, and inhibit an immune response to the cancer cells. Downregulation of T cell activation and cytokine secretion upon binding to PD-1 has been observed in several human cancers (Freeman et al., J Exp Med, 2000, 192(7): 1027-34; Latchman et al., Nat Immunol, 2001, 2(3):261-8, hereby entirely incorporated by reference). In addition, the PD-1 ligand PD-L1 is overexpressed in many cancers, including breast cancer, colon cancer, esophageal cancer, gastric cancer, glioma, leukemia, lung cancer, melanoma, multiple myeloma, ovarian cancer, pancreatic cancer, renal cell carcinoma, and urothelial cancer. It has also been shown that patients with cancer have a limited or reduced adaptive immune response due to increased PD-1/PD-L1 interactions by immune cells. This increase in activated PD-1 signaling has also been seen in patients with viral infections. For instance, hepatitis B and C viruses can induce overexpression of PD-1 ligands on hepatocytes and activate PD-1 signaling in effector T-cells. This, in turn, leads to T-cell exhaustion and immune tolerance to the viral infection (Boni et al., J Virol, 2007, 81:4215-4225; Golden-Mason et al., J Immunol, 2008, 180:3637-3641, hereby entirely incorporated by reference).

There is a need in the art for effective protein-based therapeutic agents and methods to promote T cell and/or NK cell cytotoxicity, modulate the activity of the innate and/or adaptive immune system, and/or alleviate or reverse the inhibition of adaptive immunity in patients with cancer or infection. The present invention satisfies this and other needs.

The present disclosure relates to bispecific Fc fusion proteins comprising sPD-I variant, IL-15 and IL-15Rα sushi domain having improved properties (e.g., increased binding affinity for PD-L1 and/or PD-L2, enhanced agonist activity of IL-15, extended half-life, and synergistic efficacy for treating cancer and/or infections) as well as methods of making and using such bispecific Fc fusion proteins in treating patients with cancers, infections and immune-related diseases.

### III. BRIEF SUMMARY

The present disclosure provides, inter alia, bispecific Fc fusion proteins comprising an IL-15 domain, an IL-15Rα sushi domain, an Fc domain and sPD-1 variant domain, polynucleotides encoding the bispecific Fc fusion proteins, methods of making the bispecific Fc fusion proteins, and methods of using the bispecific Fc fusion proteins, for example, in treating diseases in which the adaptive immune system is suppressed or an increase in the magnitude or level of immune response is needed (e.g., cancers, infections, etc.), modulating immune functions, promoting T cell and/or NK cell cytotoxicity, etc.

In one aspect, the present disclosure provides a bispecific Fc fusion protein comprising:
a) an IL-15Rα sushi domain;
b) an IL-15 domain;
c) an Fc domain; and
d) a soluble PD-I (sPD-1) variant domain.

In an additional aspect, the present disclosure provides the Fc fusion protein as disclosed herein, where the Fc fusion protein further comprises a first domain linker, a second domain linker, and a third domain linker.

In a further aspect, the present disclosure provides the Fc fusion protein as disclosed herein, wherein the first domain linker, the second domain linker, and the third domain linker are selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, (GS)n, (GSGGS)n, (GGGGS)n, and (GGGS)n, wherein n is selected from the group consisting of 1, 2, 3, 4 and 5.

In an additional aspect, the present disclosure provides the Fc fusion protein as disclosed herein, wherein the first domain linker is selected from the group consisting of SEQ ID NO:12, SEQ ID NO:13, SEQ ID NO:14, SEQ ID NO:15, SEQ ID NO:16, SEQ ID NO:17, SEQ ID NO: 18, SEQ ID NO:19, SEQ ID NO:20, and SEQ ID NO:21.

In a further aspect, the present disclosure provides the Fc fusion protein as disclosed herein, wherein the second domain linker is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

In an additional aspect, the present disclosure provides the Fc fusion protein as disclosed herein, wherein the third domain linker is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

In a further aspect, the present disclosure provides the Fc fusion protein as disclosed herein, wherein the Fc fusion protein comprises, from N- to C-terminus:
a) the IL-15Rα sushi domain;
b) the first domain linker;
c) the IL-15 domain;
d) the second domain linker;
e) the Fc domain;
f) the third domain linker; and
g) the sPD-1 variant domain

In an additional aspect, the present disclosure provides the Fc fusion protein as disclosed herein, wherein the Fc fusion protein comprises, from N- to C-terminus:
a) the IL-15 domain;
b) the first domain linker;
c) the IL-15Rα sushi domain;
d) the second domain linker;
e) the Fc domain;
f) the third domain linker; and
g) the sPD-1 variant domain.

In a further aspect, the present disclosure provides the Fc fusion protein as disclosed herein, wherein the Fc fusion protein comprises, from N- to C-terminus:
a) the sPD-1 variant domain;
b) the first domain linker;
c) the Fc domain;
d) the second domain linker;
e) the IL-15 domain;
f) the third domain linker; and
g) the IL-15Rα sushi domain.

In an additional aspect, the present disclosure provides the Fc fusion protein as disclosed herein, wherein the Fc fusion protein comprises, from N- to C-terminus:
a) the sPD-1 variant domain;
b) the first domain linker;
c) the Fc domain;
d) the second domain linker;
e) the IL-15Rα sushi domain;
f) the third domain linker; and
g) the IL-15 domain.

In a further aspect, the present disclosure provides the Fc fusion protein as disclosed herein, wherein the Fc fusion protein comprises, from N- to C-terminus:
a) the IL-15Rα sushi domain;
b) the first domain linker;
c) the IL-15 domain;
d) the second domain linker;
e) the sPD-1 variant domain;
f) the third domain linker; and
g) the Fc domain.

In an additional aspect, the present disclosure provides the Fc fusion protein as disclosed herein, wherein the Fc fusion protein comprises, from N- to C-terminus:
a) the IL-15 domain;
b) the first domain linker;
c) the IL-15Rα sushi domain;
d) the second domain linker;
e) the sPD-1 variant domain;
f) the third domain linker; and
g) the Fc domain.

In a further aspect, the present disclosure provides the Fc fusion protein as disclosed herein, wherein the Fc fusion protein comprises, from N- to C-terminus:
a) the sPD-1 variant domain;
b) the first domain linker;
c) the IL-15 domain;
d) the second domain linker;
e) the IL-15Rα sushi domain;
f) the third domain linker; and
g) the Fc domain.

In an additional aspect, the present disclosure provides the Fc fusion protein as disclosed herein, wherein the Fc fusion protein comprises, from N- to C-terminus:
a) the sPD-1 variant domain;
b) the first domain linker;
c) the IL-15Rα sushi domain;
d) the second domain linker;
e) the IL-15 domain;
f) the third domain linker; and
g) the Fc domain.

In a further aspect, the present disclosure provides the Fc fusion protein as disclosed herein, wherein the Fc fusion protein comprises, from N- to C-terminus:
a) the Fc domain;
b) the first domain linker;
c) the IL-15 domain;
d) the second domain linker;
e) the IL-15Rα sushi domain;
f) the third domain linker; and
g) the sPD-1 variant domain

In an additional aspect, the present disclosure provides the Fc fusion protein as disclosed herein, wherein the Fc fusion protein comprises, from N- to C-terminus:
a) the Fc domain;
b) the first domain linker;
c) the IL-15Rα sushi domain;
d) the second domain linker;
e) the IL-15 domain;
f) the third domain linker; and
g) the sPD-1 variant domain.

In a further aspect, the present disclosure provides the Fc fusion protein as disclosed herein, wherein the Fc fusion protein comprises, from N- to C-terminus:
a) the Fc domain;
b) the first domain linker;
c) the sPD-1 variant domain;
d) the second domain linker;
e) the IL-15 domain;
f) the third domain linker; and
g) the IL-15Rα sushi domain.

In an additional aspect, the present disclosure provides the Fc fusion protein as disclosed herein, wherein the Fc fusion protein comprises, from N- to C-terminus:
a) the Fc domain;
b) the first domain linker;
c) the sPD-1 variant domain;
d) the second domain linker;
e) the IL-15Rα sushi domain;
f) the third domain linker; and
g) the IL-15 domain.

In a further aspect, the present disclosure provides the Fc fusion protein as disclosed herein, wherein the domain linker linking the IL-15 domain and the IL-15Rα sushi domain is selected from the group consisting of SEQ ID NO:13, SEQ ID NO:14, SEQ ID NO:15, SEQ ID NO:16, SEQ ID NO:17, and SEQ ID NO:18; and wherein the other two domain linkers are selected from the group consisting of SEQ ID NO:19, SEQ ID NO:20, and SEQ ID NO:21.

In an additional aspect, the present disclosure provides the Fc fusion protein as disclosed herein, wherein the domain linker linking the IL-15 domain and the IL-15Rα sushi domain has the amino acid sequence of SEQ ID NO:15; and wherein the other two domain linkers are selected from the group consisting of SEQ ID NO:19, SEQ ID NO:20, and SEQ ID NO:21.

In a further aspect, the present disclosure provides the Fc fusion protein as disclosed herein, wherein the domain linker linking the IL-15 domain and the IL-15Rα sushi domain has the amino acid sequence of SEQ ID NO:18; and wherein the other two domain linkers are selected from the group consisting of SEQ ID NO:19, SEQ ID NO:20, and SEQ ID NO:21.

In an additional aspect, the present disclosure provides the Fc fusion protein as disclosed herein, wherein the sPD-1 variant domain comprises one or more amino acid substitutions at positions corresponding to positions selected from the group consisting of positions 38, 63, 65, 92, 100, 103, 108 and 116 of SEQ ID NO: 1.

In a further aspect, the present disclosure provides the Fc fusion protein as disclosed herein, wherein the sPD-1 variant domain comprises an amino acid substitution at a position corresponding to position 38 of SEQ ID NO: 1.

In an additional aspect, the present disclosure provides the Fc fusion protein as disclosed herein, wherein the sPD-1 variant domain comprises an amino acid substitution at a position corresponding to position 63 of SEQ ID NO: 1.

In a further aspect, the present disclosure provides the Fc fusion protein as disclosed herein, wherein the sPD-1 variant domain comprises an amino acid substitution at a position corresponding to position 65 of SEQ ID NO: 1.

In an additional aspect, the present disclosure provides the Fc fusion protein as disclosed herein, wherein the sPD-1 variant domain comprises an amino acid substitution at a position corresponding to position 92 of SEQ ID NO: 1.

In a further aspect, the present disclosure provides the Fc fusion protein as disclosed herein, wherein the sPD-1 variant domain comprises an amino acid substitution at a position corresponding to position 100 of SEQ ID NO: 1.

In an additional aspect, the present disclosure provides the Fc fusion protein as disclosed herein, wherein the sPD-1 variant domain comprises an amino acid substitution at a position corresponding to position 103 of SEQ ID NO: 1.

In a further aspect, the present disclosure provides the Fc fusion protein as disclosed herein, wherein the sPD-1 variant domain comprises an amino acid substitution at a position corresponding to position 108 of SEQ ID NO:1.

In an additional aspect, the present disclosure provides the Fc fusion protein as disclosed herein, wherein the sPD-1 variant domain comprises an amino acid substitution at a position corresponding to position 116 of SEQ ID NO:1.

In a further aspect, the present disclosure provides the Fc fusion protein as disclosed herein, wherein said one or more amino acid substitutions occur at two of said positions, three of said positions, four of said positions, five of said positions, six of said positions, seven of said positions or eight of said positions.

In an additional aspect, the present disclosure provides the Fc fusion protein as disclosed herein, wherein the sPD-1 variant domain comprises an amino acid sequence having at least 96% sequence identity to SEQ ID NO:1.

In a further aspect, the present disclosure provides the Fc fusion protein as disclosed herein, wherein the sPD-1 variant domain comprises one or more amino acid substitutions selected from the group consisting of S38G, S63G, P65L, N92S, G100S, S103V, A1081, and Al16V of SEQ ID NO:1.

In an additional aspect, the present disclosure provides the Fc fusion protein as disclosed herein, wherein the sPD-1 variant domain comprises a set of amino acid substitutions N92S/G100S/S103V/A1081/A116V of SEQ ID NO:1.

In a further aspect, the present disclosure provides the Fc fusion protein as disclosed herein, wherein the sPD-1 variant domain comprises a set of amino acid substitutions S38G/S63G/P65L/N92S/G100S/S103V/A108I/A116V of SEQ ID NO:1.

In an additional aspect, the present disclosure provides the Fc fusion protein as disclosed herein, wherein the sPD-1 variant domain comprises a set of amino acid substitutions S38G/S63G/P65L/G100S/S103V/A108I/A116V of SEQ ID NO:1.

In a further aspect, the present disclosure provides the Fc fusion protein as disclosed herein, wherein the sPD-1 variant domain comprises a set of amino acid substitutions P65L/G100S/S103V/A1081/A116V of SEQ ID NO:1.

In an additional aspect, the present disclosure provides the Fc fusion protein as disclosed herein, wherein the sPD-1 variant domain comprises a set of amino acid substitutions S63G/G100S/S103V/A1081/A116V of SEQ ID NO:1.

In a further aspect, the present disclosure provides the Fc fusion protein as disclosed herein, wherein the sPD-1 variant domain comprises a set of amino acid substitutions S63G/P65L/G100S/S103V/A108I/A116V of SEQ ID NO:1.

In an additional aspect, the present disclosure provides the Fc fusion protein as disclosed herein, wherein the sPD-1 variant domain comprises a set of amino acid substitutions G100S/S103V/A1081/A116V of SEQ ID NO:1.

In a further aspect, the present disclosure provides the Fc fusion protein as disclosed herein, wherein the sPD-1 variant domain comprises a set of amino acid substitutions G100S/S103V/A108I of SEQ ID NO:1.

In an additional aspect, the present disclosure provides the Fc fusion protein as disclosed herein, wherein the sPD-1 variant domain comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8 and SEQ ID NO:9.

In a further aspect, the present disclosure provides the Fc fusion protein as disclosed herein, wherein the sPD-1 variant domain comprises the amino acid sequence of SEQ ID NO:7.

In an additional aspect, the present disclosure provides the Fc fusion protein as disclosed herein, wherein the IL-15 domain comprises the amino acid sequence of SEQ ID NO:10.

In a further aspect, the present disclosure provides the Fc fusion protein as disclosed herein, wherein the IL-15Rα sushi domain comprises the amino acid sequence of SEQ ID NO:11.

In an additional aspect, the present disclosure provides the Fc fusion protein as disclosed herein, wherein the Fc domain is a human IgG Fc domain or a variant human IgG Fc domain.

In a further aspect, the present disclosure provides the Fc fusion protein as disclosed herein, wherein the human IgG Fc domain comprises hinge-CH2-CH3 of human IgG4.

In an additional aspect, the present disclosure provides the Fc fusion protein as disclosed herein, wherein the Fc domain is a variant human IgG Fc domain.

In a further aspect, the present disclosure provides the Fc fusion protein as disclosed herein, wherein said variant human IgG Fc domain comprises hinge-CH2-CH3 of human IgG4 with a substitution corresponding to S228P as set forth in SEQ ID NO:25.

In an additional aspect, the present disclosure provides the Fc fusion protein comprising the amino acid sequence of SEQ ID NO:26.

In a further aspect, the present disclosure provides the Fc fusion protein comprising the amino acid sequence of SEQ ID NO:27.

In an additional aspect, the present disclosure provides the Fc fusion protein comprising the amino acid sequence of SEQ ID NO:28.

In a further aspect, the present disclosure provides the Fc fusion protein comprising the amino acid sequence of SEQ ID NO:29.

In an additional aspect, the present disclosure provides the Fc fusion protein comprising the amino acid sequence of SEQ ID NO:30.

In a further aspect, the present disclosure provides the Fc fusion protein comprising the amino acid sequence of SEQ ID NO:31.

In an additional aspect, the present disclosure provides the Fc fusion protein comprising the amino acid sequence of SEQ ID NO:32.

In a further aspect, the present disclosure provides the Fc fusion protein comprising the amino acid sequence of SEQ ID NO:33.

In an additional aspect, the present disclosure provides the Fc fusion protein comprising the amino acid sequence of SEQ ID NO:34.

In a further aspect, the present disclosure provides the Fc fusion protein comprising the amino acid sequence of SEQ ID NO:35.

In an additional aspect, the present disclosure provides the Fc fusion protein comprising the amino acid sequence of SEQ ID NO:36.

In a further aspect, the present disclosure provides the Fc fusion protein comprising the amino acid sequence of SEQ ID NO:37.

In an additional aspect, the present disclosure provides the Fc fusion protein comprising the amino acid sequence of SEQ ID NO:62.

In a further aspect, the present disclosure provides the Fc fusion protein comprising the amino acid sequence of SEQ ID NO:63.

In an additional aspect, the present disclosure provides the Fc fusion protein comprising the amino acid sequence of SEQ ID NO:64.

In a further aspect, the present disclosure provides the Fc fusion protein comprising the amino acid sequence of SEQ ID NO:65.

In an additional aspect, the present disclosure provides the Fc fusion protein comprising the amino acid sequence of SEQ ID NO:66.

In a further aspect, the present disclosure provides the Fc fusion protein comprising the amino acid sequence of SEQ ID NO:68.

In an additional aspect, the present disclosure provides the Fc fusion protein comprising the amino acid sequence of SEQ ID NO:69.

In a further aspect, the present disclosure provides the Fc fusion protein comprising the amino acid sequence of SEQ ID NO:70.

In an additional aspect, the present disclosure provides the Fc fusion protein comprising the amino acid sequence of SEQ ID NO:71.

In a further aspect, the present disclosure provides the Fc fusion protein comprising the amino acid sequence of SEQ ID NO:72.

In an additional aspect, the present disclosure provides a pharmaceutical composition comprising the Fc fusion protein as disclosed herein and a pharmaceutically acceptable carrier, excipient and/or stabilizer.

In a further aspect, the present disclosure provides a nucleic acid encoding the Fc fusion protein as disclosed herein.

In an additional aspect, the present disclosure provides the nucleic acid encoding the Fc fusion protein as disclosed herein, wherein the nucleic acid is codon optimized for a host organism for expression of the Fc fusion protein in said organism.

In a further aspect, the present disclosure provides an expression vector comprising the nucleic acid as disclosed herein.

In an additional aspect, the present disclosure provides a method of making the bispecific Fc fusion protein as disclosed herein comprising: a) culturing the host cell as disclosed herein under conditions wherein said Fc fusion protein is expressed; and b) recovering said Fc fusion protein.

In a further aspect, the present disclosure provides a nucleic acid encoding a preprotein comprising a signal peptide and the Fc fusion protein as disclosed herein.

In an additional aspect, the present disclosure provides the nucleic acid encoding a preprotein comprising a signal peptide and the Fc fusion protein as disclosed herein, wherein the signal peptide comprises an amino acid sequence having at least 85% sequence identity to SEQ ID NO:22 or SEQ ID NO:23.

In a further aspect, the present disclosure provides the nucleic acid encoding a preprotein comprising a signal peptide and the Fc fusion protein as disclosed herein, wherein the signal peptide comprises the amino acid sequence of SEQ ID NO:22.

In an additional aspect, the present disclosure provides the nucleic acid encoding a preprotein comprising a signal peptide and the Fc fusion protein as disclosed herein, wherein the signal peptide comprises the amino acid sequence of SEQ ID NO:23.

In a further aspect, the present disclosure provides the nucleic acid encoding the preprotein as disclosed herein, wherein the preprotein comprises an amino acid sequence selected from the group consisting of SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82 and SEQ ID NO:83.

In an additional aspect, the present disclosure provides an expression vector comprising the nucleic acid as disclosed herein.

In a further aspect, the present disclosure provides a host cell comprising the nucleic acid as disclosed herein, or the expression vector as disclosed herein.

In an additional aspect, the present disclosure provides a method of making a bispecific Fc fusion protein comprising: a) culturing the host cell as disclosed herein under conditions wherein said Fc fusion protein is expressed; and b) recovering said Fc fusion protein.

In a further aspect, the present disclosure provides a method of treating, reducing or preventing metastasis or invasion of a tumor in a subject with cancer, the method comprising administering to the subject a therapeutically effective dose of one or more said Fc fusion proteins as disclosed herein or the pharmaceutical composition as disclosed herein.

In an additional aspect, the present disclosure provides the method of treating, reducing or preventing metastasis or invasion of a tumor in a subject with cancer as disclosed herein, wherein the tumor is a solid tumor.

In a further aspect, the present disclosure provides the method of treating, reducing or preventing metastasis or invasion of a tumor in a subject with cancer as disclosed herein, wherein the cancer is a colorectal cancer.

In an additional aspect, the present disclosure provides the method of treating, reducing or preventing metastasis or invasion of a tumor in a subject with cancer as disclosed herein, wherein the effective dose of the one or more Fc fusion proteins or the pharmaceutical composition inhibits, reduces, or modulates signal transduction mediated by the wild-type PD-1 in the subject.

In a further aspect, the present disclosure provides the method of treating, reducing or preventing metastasis or invasion of a tumor in a subject with cancer as disclosed herein, wherein the effective dose of the one or more Fc fusion proteins or the pharmaceutical composition increases a T cell response in the subject.

In an additional aspect, the present disclosure provides a method of preventing or treating an infection in a subject, the method comprising administering to the subject a therapeutically effective dose of one or more said Fc fusion proteins as disclosed herein or the pharmaceutical composition as disclosed herein.

In a further aspect, the present disclosure provides the method of preventing or treating an infection in a subject as disclosed herein, wherein the infection is selected from the group consisting of a fungal infection, bacterial infection and viral infection.

In an additional aspect, the present disclosure provides the method of preventing or treating an infection in a subject as disclosed herein, wherein the effective dose of the one or more Fc fusion proteins or the pharmaceutical composition inhibits, reduces, or modulates signal transduction mediated by the wild-type PD-1 in the subject.

In a further aspect, the method of preventing or treating an infection in a subject as disclosed herein, wherein the effective dose of the one or more Fc fusion proteins or the pharmaceutical composition increases a T cell response in the subject.

In an additional aspect, the present disclosure provides a method of preventing or treating an IL-15 mediated disease or disorder in a subject, the method comprising administering to the subject a therapeutically effective dose of one or more said Fc fusion proteins as disclosed herein or the pharmaceutical composition as disclosed herein, wherein the IL-15 mediated disease or disorder is a cancer or an infectious disease.

In an additional aspect, the present disclosure provides the method of preventing or treating an IL-15 mediated disease or disorder in a subject as disclosed herein, wherein the cancer is colorectal cancer.

In a further aspect, the present disclosure provides the method of preventing or treating an IL-15 mediated disease or disorder in a subject as disclosed herein, wherein the infectious disease is a viral infection.

In an additional aspect, the present disclosure provides a method of preventing or treating an immunodeficiency or lymphopenia in a subject, comprising administering to the subject a therapeutically effective dose of one or more said Fc fusion proteins as disclosed herein or said pharmaceutical composition as disclosed herein.

In a further aspect, the present disclosure provides a method of enhancing IL-15-mediated immune function in a subject in need thereof. comprising administering to the subject a therapeutically effective dose of one or more said Fc fusion proteins as disclosed herein or said pharmaceutical composition as disclosed herein.

In an additional aspect, the present disclosure provides the method of enhancing IL-15-mediated immune function in a subject in need thereof as disclosed herein, wherein the enhanced IL-15-mediated immune function comprises proliferation of lymphocytes, inhibition of apoptosis of lymphocytes, antibody production, activation of antigen presenting cells and/or antigen presentation.

In a further aspect, the present disclosure provides the method of enhancing IL-15-mediated immune function in a subject in need thereof as disclosed herein, wherein the enhanced IL-15-mediated immune function comprises activation or proliferation of CD4+ T cells, CD8+ T cells, B cells, memory T cells, memory B cells, dendritic cells, other antigen presenting cells, macrophages, mast cells, natural killer T cells (NKT cells), tumor-resident T cells, CD122+ T cells, and/or natural killer cells (NK cells).

In an additional aspect, the present disclosure provides a method of promoting T cell cytotoxicity or NK cell cytotoxicity in a subject in need thereof, comprising administering to the subject a therapeutically effective dose of one or more said Fc fusion proteins as disclosed herein or said pharmaceutical composition as disclosed herein.

### IV. BRIEF DESCRIPTION OF THE DRAWINGS

FIG. 1 provides flowchart illustrations of molecule cell line development including culture medium and selection drug used.

FIG. 2 provides a Table of pooled clones corresponding to each sequence including culture medium and selection drug used.

FIG. 3A shows results of cell viability recovery of Sequence 1 clone GI to G4.

FIG. 3B shows results of cell viability recovery of Sequence 2 clone G5 to G8. FIG. 3C shows results of cell viability recovery of Sequence 1-1 clone G9 to G12. FIG. 3D shows results of cell viability recovery of Sequence 2-1 clone G13 to G16.

FIG. 4 shows tabulated productivity and cell growth profile of recovered clone pools by 11 days fed-batch culture.

FIG. 5A shows results for SDS-PAGE of recovered pools by 11 days fed-batch culture using Harvested Cell Culture Fluid (HCCF), clone Gi to G8. FIG. 5B shows results for SDS-PAGE of recovered pools by 11 days fed-batch culture using Harvested Cell Culture Fluid (HCCF), clone G9 to G16.

FIG. 6A shows results for SDS-PAGE of recovered pools by 11 days fed-batch culture using ProA purified sample clone Gi to G8. FIG. 6B shows results for SDS-PAGE of recovered pools by 11 days fed-batch culture using ProA purified sample clone Gi to G8.

FIG. 7 shows a Table for Titer distribution of 1^{st} round single cell clones in 12 day-batch culture.

FIG. 8A shows Sequence No. 1-1 single cell clones cell growth curve. FIG. 8B shows Sequence No. 1-1 single cell clones cell viability curve.

FIG. 9A shows Sequence No. 2-1 single cell clones cell growth curve. FIG. 9B shows Sequence No. 2-1 single cell clones cell viability curve.

FIG. 10 shows a Table for Titer distribution of top 10 clones selected from Sequence 1-1 and 2-1.

FIG. 11 provides Tabulated summaries of pooled clones IL-15 binding potency and EC50 determined by IL-15 Receptor β binding ELISA.

FIG. 12 shows results for relative potency of IL-15 determined by IL-15 Receptor β binding ELISA.

FIG. 13 shows results for EC50 of IL-15 determined by IL-15 Receptor β binding ELISA.

FIG. 14 provides tabulated summaries of pooled clones PD-L1 binding potency and EC50 determined by PD-L1 binding ELISA.

FIG. 15A shows results for relative potency of PD-L1 binding determined by PD-L1 binding ELISA. FIG. 15B provides results for EC50 of PD-L1 determined by PD-L1 binding ELISA.

FIG. 16 provides tabulated summaries of pooled clones PD-L2 binding potency and EC50 determined by PD-L2 binding ELISA.

FIG. 17A shows results for relative potency of PD-L1 binding determined by PD-L1 binding ELISA. FIG. 17B shows results for EC50 of PD-L1 determined by PD-L1 binding ELISA.

FIG. 18 shows results for final tumor volume analysis of MC38-hPD-L1 colorectal tumors treated with 0.1 mg/kg, 1 mg/kg and 10 mg/kg of sPD1/IL-15 molecule comparing to tumor-bearing animals treated with sPD-1 treatment at 10 mg/kg, single IL-15 treatment at 2.5 mg/kg and sPD-1 and IL-15 combined treatment. Inlaid graph shows the comparison between sPD-1 and IL-15 combined treatment comparing to sPD-1/IL-15 at 1 mg/kg and 10 mg/kg.

FIG. 19A is a table that summarizes the treatment groups, dose concentrations, and dose volumes used in an in vivo study comparing the antitumor activities of AB002 (SEQ ID NO: 97) and other PD-1 immune checkpoint inhibitors in combination with 11-15 agonist. FIG. 19B shows the dosing schedule for the same in vivo study.

FIGS. 20 items A and B show the changes in tumor volume in the different treatment groups at the end of the in vivo study explained in FIG. 19C. FIG. 20 item C shows the changes in body weight of the subject mice in the same in vivo study.

FIG. 21 shows changes in the gene expression levels of target genes in MC38 tumor cells after treatment with AB002 (SEQ ID NO: 97) or mouse aPD-1 antibody.

FIG. 22 item A is a table summarizing the summarizes the treatment groups, dose concentrations, and dose volumes used in an in vivo study comparing the effect of AB002 (SEQ ID NO: 97) and anti-mPD1 on mice injected with Lewis lung tumor cells. FIG. 22 item B shows percent inhibition of tumor volume following treatment by AB002 (SEQ ID NO: 97) or Anti-mPD-1 in the in vivo study conducted according to FIG. 22 item A.

FIG. 23 item A and FIG. 23 item B show the changes in tumor volume from an in vivo study of mice inoculated with MC38 tumor cells when treated either intravenously or subcutaneously with different doses of AB002 (SEQ ID NO: 97). In addition, changes in tumor volume when different doses of sPD-1 equivalent to the doses of AB002 (SEQ ID NO: 97) administered in intravenously or subcutaneously dosing. FIG. 23 item C and FIG. 23D show effectiveness of tumor treatment by AB002 (SEQ ID NO: 97) alone or in combination with aPD-L1 Ab.

FIG. 24 shows amino acid sequences of Wild Type ECD of Human PD-1 without the First Four Amino Acids (SEQ ID NO:1) and variant ECD of Human PD-1 without the first four amino acids (SEQ ID NO. 2 to SEQ ID NO. 9).

FIG. 25 shows amino acid sequences of human IL-15 domain (SEQ ID NO:10), human IL-15R alpha sushi domain (SEQ ID NO:11), Linker (SEQ ID NO:12), Linker variant 1 (SEQ ID NO:13), Linker variant 2 (SEQ ID NO:14), Linker variant 3 (SEQ ID NO:15), Linker variant 4 (SEQ ID NO:16), Linker variant 5 (SEQ ID NO:17), Linker variant 6 (SEQ ID NO:18), GS Linker (SEQ ID NO:19), GS Linker X2 (SEQ ID NO:20), GS Linker X3 (SEQ ID NO:21), Signal Peptide 1(SEQ ID NO:22) and Signal Peptide 2 (SEQ ID NO.23).

FIG. 26 shows amino acid sequence of Human IgG4 (SEQ ID NO:24) and variant Human IgG4 (SEQ ID NO:25).

FIG. 27A shows amino acid sequences of fusion protein 1 with Linker variant 1 and GS linker X2 (SEQ ID NO:26) and fusion protein 2 with Linker variant 2 and GS linker X2 (SEQ ID NO:27). FIG. 27B shows amino acid sequences of fusion protein 3 with Linker variant 3 and GS linker X2 (SEQ ID NO:28) and fusion protein 4 with Linker variant 4 and GS linker X2 (SEQ ID NO:29). FIG. 27C shows amino acid sequences of fusion protein 5 with Linker variant 5 and GS linker X2 (SEQ ID NO:30) and fusion protein 6 with Linker variant 6 and GS linker X2 (SEQ ID NO:31). FIG. 27D shows amino acid sequences of fusion protein 7 with Linker variant 1 and GS linker X3 (SEQ ID NO:32) and fusion protein 8 with Linker variant 2 and GS linker X3 (SEQ ID NO:33). FIG. 27E shows amino acid sequences of fusion protein 9 with Linker variant 3 and GS linker X3 (SEQ ID NO:34) and fusion protein 10 with Linker variant 4 and GS linker X3 (SEQ ID NO:35). FIG. 27F shows amino acid sequence of fusion protein 11 with Linker variant 5 and GS linker X3 (SEQ ID NO:36) and fusion protein 12 with Linker variant 6 and GS linker X3 (SEQ ID NO:37).

FIG. 28A shows amino acid sequences of preprotein 1 including signal peptide 1 and fusion protein 1 (SEQ ID NO: 38), and preprotein 2 including signal peptide 1 and fusion protein 2 (SEQ ID NO: 39). FIG. 28B shows amino acid sequences of preprotein 3 including signal peptide 1 and fusion protein 3 (SEQ ID NO: 40), and preprotein 4 including signal peptide 1 and fusion protein 4 (SEQ ID NO: 41). FIG. 28C shows amino acid sequences of preprotein 5 including signal peptide 1 and fusion protein 5 (SEQ ID NO: 42), and preprotein 6 including signal peptide 1 and fusion protein 6 (SEQ ID NO: 43). FIG. 28D shows amino acid sequences of preprotein 7 including signal peptide 1 and fusion protein 7 (SEQ ID NO: 44), and preprotein 8 including signal peptide 1 and fusion protein 8 (SEQ ID NO: 45). FIG. 28E shows amino acid sequences of preprotein 9 including signal peptide 1 and fusion protein 9 (SEQ ID NO: 46), and preprotein 10 including signal peptide 1 and fusion protein 10 (SEQ ID NO: 47). FIG. 28F shows amino acid sequences of preprotein 11 including signal peptide 1 and fusion protein 11 (SEQ ID NO: 48), and preprotein 12 including signal peptide 1 and fusion protein 12 (SEQ ID NO: 49).

FIG. 29A shows amino acid sequences of preprotein 13 including signal peptide 2 and fusion protein 1 (SEQ ID NO: 50), and preprotein 14 including signal peptide 2 and fusion protein 2 (SEQ ID NO: 51). FIG. 29B shows amino acid sequences of preprotein 15 including signal peptide 2 and fusion protein 3 (SEQ ID NO: 52), and preprotein 16 including signal peptide 2 and fusion protein 4 (SEQ ID NO: 53). FIG. 29C shows amino acid sequences of preprotein 17 including signal peptide 2 and fusion protein 5 (SEQ ID NO: 54), and preprotein 18 including signal peptide 2 and fusion protein 6 (SEQ ID NO: 55). FIG. 29D shows amino acid sequences of preprotein 19 including signal peptide 2 and fusion protein 7 (SEQ ID NO: 56), and preprotein 20 including signal peptide 2 and fusion protein 8 (SEQ ID NO: 57). FIG. 29E shows amino acid sequences of preprotein 21 including signal peptide 2 and fusion protein 9 (SEQ ID NO: 58), and preprotein 22 including signal peptide 2 and fusion protein 10 (SEQ ID NO: 59). FIG. 29F shows amino acid sequences of preprotein 23 including signal peptide 2 and fusion protein 11 (SEQ ID NO: 60), and preprotein 24 including signal peptide 2 and fusion protein 12 (SEQ ID NO: 61).

FIG. 30A shows amino acid sequences of fusion protein 6A (SEQ ID NO: 62) and fusion protein 6B (SEQ ID NO: 63). FIG. 30B shows amino acid sequences of fusion protein 6C (SEQ ID NO: 64) and fusion protein 6D (SEQ ID NO: 65). FIG. 30C shows amino acid sequences of fusion protein 6E (SEQ ID NO: 66) and fusion protein 6F (SEQ ID NO: 67). FIG. 30D shows amino acid sequences of fusion protein 6G (SEQ ID NO: 68) and fusion protein 6H (SEQ ID NO: 69). FIG. 30E shows amino acid sequences of fusion protein 61 (SEQ ID NO: 70) and fusion protein 6J (SEQ ID NO: 71). FIG. 30F shows amino acid sequences of fusion protein 6K (SEQ ID NO: 72).

FIG. 31A shows amino acid sequences of preprotein 18A (SEQ ID NO: 73) and preprotein 18B (SEQ ID NO: 74). FIG. 31B shows amino acid sequences of preprotein 18C (SEQ ID NO: 75) and preprotein 18D (SEQ ID NO: 76). FIG. 31C shows amino acid sequences of preprotein 18E (SEQ ID NO: 77) and preprotein 18F (SEQ ID NO: 78). FIG. 31D shows amino acid sequences of preprotein 18G (SEQ ID NO: 79) and preprotein 18G (SEQ ID NO: 79). FIG. 31E shows amino acid sequences of preprotein 181 (SEQ ID NO: 81) and preprotein 18J (SEQ ID NO: 82). FIG. 31F shows amino acid sequences of preprotein 18K (SEQ ID NO: 83).

FIG. 32A shows DNA sequence encoding preprotein 1 (SEQ ID NO: 84). FIG. 32B shows DNA sequence encoding preprotein 2 (SEQ ID NO: 85). FIG. 32C shows DNA sequence encoding preprotein 3 (SEQ ID NO: 86). FIG. 32D shows DNA sequence encoding preprotein 7 (SEQ ID NO: 87). FIG. 32E shows DNA sequence encoding preprotein 8 (SEQ ID NO: 88). FIG. 32F shows DNA sequence encoding preprotein 9 (SEQ ID NO: 89). FIG. 32G shows DNA sequence encoding preprotein 13 (SEQ ID NO: 90). FIG. 32H shows DNA sequence encoding preprotein 14 (SEQ ID NO: 91). FIG. 32I shows DNA sequence encoding preprotein 15 (SEQ ID NO: 92). FIG. 32J shows DNA sequence encoding preprotein 19 (SEQ ID NO: 93). FIG. 32K shows DNA sequence encoding preprotein 20 (SEQ ID NO: 94). FIG. 32L shows DNA sequence encoding preprotein 21 (SEQ ID NO: 95).

FIG. 33 shows the amino acid sequence of wild-type ECD of Human PD-1 (SEQ ID NO:96).

FIG. 34 shows an exemplary AB002 sequence.

### V. DETAILED DESCRIPTION

### A. Introduction

IL-15 is a cytokine of about 12-14 KD and plays an important role in the development, proliferation and activation of T cells and NK cells. IL-15 can promote both innate and adaptive immune reactions by stimulating CD8+/CD4+ T cells and NK cells while showing no effect in activating T-regulatory (Treg) cells or inducing activation-associated death among effector T cells and NK cells (Q. Hu et al. Scientific Reports, 2018, 8 (7675): 1-11, hereby entirely incorporated by reference).

IL-15 binds to the interleukin-15 receptor (IL-15R) which consists of α, β, and γc chains. IL-15Rβ (also known as IL-2Rβ or CD122) and IL-15Rγc (also known as CD132) can bind to both IL-15 and IL-2 with intermediate affinity. IL-15Rα is widely expressed and only binds to IL-15 with high affinity. IL-15Rα contains a sushi domain (1-65 amino acids), which is responsible for interacting with IL-15, and is essential for mediating the biological function of IL-15 (Q. Hu et al. Scientific Reports, 2018, 8 (7675): 1-11, hereby entirely incorporated by reference).

PD-1 is an inhibitory cell surface receptor involved in controlling T-cell function during immunity and tolerance. Upon binding to its ligand, e.g., PD-L1 or PD-L2, PD-1 inhibits T-cell effector functions. The structure of PD-1 is of a single-pass type 1 membrane protein. PD-1 is encoded by the programmed cell death 1 receptor gene (Entrez Gene ID: 5133). The human PD-1 mRNA (coding) sequence is set forth in, e.g., Genbank Accession No. NM 005018. The human PD-1 polypeptide sequence is set forth in, e.g., Genbank Accession No. NP_005009 or UniProt No. Q15116. PD-1 is also known as programmed cell death 1, PDCD1, PD1, CD279, SLEB2, hPD-1, and hSLE-1. The wild-type human PD-1 polypeptide is 288 amino acids.

As is known in the art, there are a number of therapeutic antibodies that bind to PD-1 to block the binding of PD-1 to either the PD-L1 or PD-L2 receptor to result in the reduction of immune suppression to effect immune activation. These antibodies include KEYTRUDA® and OPDIVO®, as well as a number of others being tested in the clinic. Similarly, there are anti-PD-Li antibodies that are approved to result in a similar mechanism and therapeutic effect, such as TECENTRIQ®.

The present disclosure is directed to a novel mechanism of using the ECD of human PD-1, including variants, to effect similar biological function and therapeutic effect in combination of using IL-15 and IL-15Rα to enhance the agonist activity of IL-15. Thus, the present disclosure provides bispecific fusion proteins. The fusion proteins described herein comprise four general functional components. The first component comprises variants of the soluble, ECD of human PD-1 (“sPD-1 variant” hereinafter). The sPD-1 variants serve to increase the binding affinity for PD-L1 and/or PD-L2 and/or the protein stability. The second component is IL-15 domain, and the third domain is IL-15Rα sushi domain that is linked to the IL-15 domain via a domain linker. IL-15 Rα sushi domain would bind IL-15 to mediate the biological function of IL-15. The fourth domain is the Fc domain of a human IgG protein, e.g., human IgG4, to confer a significant increase in half-life of the sPD-1 variant and IL-15 as a fusion protein. These four components, or domains, are generally linked using domain linkers, such as glycine-serine linkers as outlined herein, to form the bispecific Fc fusion protein of the disclosure. Therefore, the present disclosure provides compositions and methods for modulating PD-1 and/or IL-15 mediated signaling pathway, such as stimulating the development, proliferation and activation of T cells and/or NK cells, and/or reducing T cell inhibitory signals in patients with cancers or infections.

### B. Definitions

As used herein, the following terms have the meanings ascribed to them unless specified otherwise.

The terms “a”, “an”, or “the” as used herein not only include aspects with one member, but also include aspects with more than one member. For instance, the singular forms “a”, “an”, and “the” include plural referents unless the context clearly dictates otherwise. Thus, for example, reference to “a cell” includes a plurality of such cells and reference to “the agent” includes reference to one or more agents known to those skilled in the art, and so forth.

As used herein, “protein” herein is meant at least two covalently attached amino acids, which includes proteins, polypeptides, oligopeptides and peptides.

“Fc fusion protein” herein is meant a bioengineered protein that joins the crystallizable fragment (Fc) domain of an antibody with another biologically active protein domain(s) or peptide(s) to generate a molecule with unique structure-function properties and significant therapeutic potential.

The term “bispecific protein”, “bispecific fusion protein”, “bifunctional protein”, “bifunctional fusion protein” or “bifunctional molecule” herein is meant a protein that can simultaneously bind two separate and unique ligands or receptors and modulate two different signaling pathways. The term “bispecific Fc fusion protein” herein is meant an Fc fusion protein that can simultaneously bind two separate and unique ligands or receptors and/or modulate two different signaling pathways. For example, the bispecific Fc fusion protein of the present disclosure comprising sPD-1 variant domain, IL-15 domain and IL-15Rα domain can bind the ligands of sPD-1 (e.g., PD-L1 and/or PD-L2) and the receptors of IL-15/IL-15Rα complex (e.g., the IL-15R βγ low-affinity receptor complex), and thus is able to induce IL-15 mediated and/or PD-1 mediated signaling pathways. In some embodiments, the Fc fusion protein may further include a signal peptide described herein.

The term “isolated” refers to a molecule that is substantially free of its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least about 70%-80%, 80%-90%, or 90%-95% (w/w) pure, or at least about 95%, 96%, 97%, 98%, 99%, or 100% (w/w) pure.

The term “ligand” refers to a biomolecule that is able to bind to and form a complex with a second biomolecule such as a receptor present on the surface of target cells to serve a biological purpose. A ligand is generally an effector molecule that binds to a site on a target protein, e.g., by intermolecular forces such as ionic bonds, hydrogen bonds, hydrophobic interactions, dipole-dipole bonds, or Van der Waals forces. The sPD-1 variant of the disclosure can bind to and form a complex with a PD-1 ligand such as PD-L1 and/or PD-L2.

The term “receptor” refers to a biomolecule present on the surface of a target cell that is able to bind to and form a complex with a second biomolecule such as a ligand. A receptor generally activates a specific signal transduction pathway. For instance, IL-15Rα is a receptor that binds IL-15. PD-L1 and PD-L2 are examples of cell surface receptors that bind PD-1.

By “position” as used herein is meant a location in the sequence of a protein. Positions may be numbered sequentially, or according to an established format, for example the EU index. In some embodiments of the present disclosure, positions are numbered sequentially starting with the first amino acid of the mature protein.

By “amino acid modification” herein is meant an amino acid substitution, insertion, and/or deletion in a polypeptide sequence.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution S228P refers to a variant polypeptide, in this case an Fc variant of human IgG4, in which the serine at position 228 is replaced with proline. For clarity, a protein which has been engineered to change the nucleic acid coding sequence but not change the starting amino acid (for example, exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233 #, E233( ) or E233del designates a deletion of glutamic acid at position 233. Additionally, EDA233− or EDA233 #designates a deletion of the sequence GluAspAla that begins at position 233.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant. In this context, a “parent Fc domain” will be relative to the recited variant; thus, a “variant human IgG Fc domain” is compared to the parent Fc domain of human IgG, for example, a “variant human IgG4 Fc domain” is compared to the parent Fc domain of human IgG4, etc.

By “wild type” or “WT” herein is meant an amino acid sequence or a nucleotide sequence that is found in nature, including allelic variations. A wild-type protein (or a WT protein) has an amino acid sequence or a nucleotide sequence that has not been intentionally modified.

By “variant protein” or “protein variant”, or “variant” as used herein is meant a protein that differs from that of a parent protein by virtue of at least one amino acid modification. In some embodiments, the parent proteins are human wild-type sequences or fragment thereof. In some embodiments, the parent proteins are human sequences with variants. Protein variant may refer to the protein itself, a composition comprising the protein, or the amino sequence that encodes it. Preferably, the protein variant has at least one amino acid modification compared to the parent protein, e.g., from about one to about twenty amino acid modifications, and preferably from about one to about eight amino acid modifications compared to the parent. The protein variant sequence herein may preferably possess at least about 80%, 81%, 82%, 83%, 84%, 85%, 86%, 87%, 88%, 89%, 90%, 91%, 92%, 93%, 94%, 95%, 96%, 97%, 98% or 99% identity with a parent protein sequence, preferably at least about 90% identity, and preferably at least about 95%, 97%, 98%, or 99% identity. The protein variant sequence herein may possess 99%, 98%, 97%, 96%, 95%, 94%, 93%, 92%, 91%, 90% or less identity with the parent protein sequence. Sequence identity between two similar sequences (e.g., sPD-1 variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al., (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

“IgG variant” or “variant IgG” as used herein is meant an antibody that differs from a parent IgG (again, in many cases, from a human IgG sequence) by virtue of at least one amino acid modification.

“Fc variant” or “variant Fc” as used herein is meant a protein comprising at least one amino acid modification as compared to a parental Fc domain. In some embodiments, the parent Fc domain, is a human wild-type Fc sequence, such as the Fc region from IgG1, IgG2, IgG3 or IgG4. Thus, a “variant human IgG4 Fc domain” is one that contains amino acid modifications (generally amino acid substitutions) as compared to the human IgG4 Fc domain. For example, S241P or S228P is a hinge variant with the substitution proline at position 228 relative to the parent IgG4 hinge polypeptide, wherein the numbering S228P is according to the EU index and the S241P is the Kabat numbering. The EU index or EU index as in Kabat or EU numbering scheme refers to the EU numbering (see SEQUENCES OF IMMUNOLOGICAL INTEREST, 5th edition, NIH publication, No. 91-3242, E. A. Kabat et al., hereby entirely incorporated by reference; and see also Edelman et al., 1969, Proc Natl Acad Sci USA 63:78-85, hereby entirely incorporated by reference). In some embodiments, the parent Fc domains are human Fc sequences with variants. For all positions discussed in the present disclosure that relate to the Fc domain of a human IgG, unless otherwise noted, amino acid position numbering is according to the EU index. The modification can be an addition, deletion, substitution or any combination thereof as outlined herein. Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains herein still retain the ability to form a dimer with another Fc domain, as well as bind to the FcRn receptor as measured using known techniques as described herein, such as non-denaturing gel electrophoresis.

The term “soluble PD-1” or “sPD-1” herein is meant a soluble portion of the programmed cell death 1 (PD-1) polypeptide containing the extracellular domain (ECD) or a fragment or truncated version thereof, but not the transmembrane domain or the cytoplasmic (intracellular) domain of PD-1. The sequence of ECD of human wild-type PD-1 is set forth as SEQ ID NO: 96. In some embodiments, the parent wild-type sPD-1 domain can have N-terminal and/or C-terminal truncations (e.g., the truncated human sPD-1 as set forth in SEQ ID NO:1) as long as the truncated wild-type sPD-1 retains biological activity, e.g., binding to PD-L1 and/or PD-L2.

The term “sPD-1 variant” refers to a variant of a wild-type sPD-1 or a fragment or truncated version thereof. The sPD-1 variant retains specific binding to a PD-1 ligand, such as PD-L1 and/or PD-L2, but has amino acid substitutions, and can have N- or C-terminal truncations as compared to wild-type sPD-1. Specific binding in this case is as determined by a standard binding assay, such as an ELISA, Biacore, Sapidyne KinExA, or Flow Cytometry binding analysis, which assays can also be used to determine binding affinity. As discussed herein, sPD-1 variants may have, in some instances, increased binding affinity as compared to wild-type sPD-1.

The term “binding affinity” refers to the ability of a ligand or variant thereof to form coordinated bonds with a protein, e.g., a receptor or a variant thereof. The binding affinity between a ligand and protein can be represented by an equilibrium dissociation constant (KD), a ratio of koff/kon between the ligand and the protein (e.g., receptor or a variant thereof). KD and binding affinity are inversely related. For instance, the KD value relates the concentration of the sPD-1 variant needed to bind to a PD-1 ligand, and a lower KD value (lower PD-1 variant concentration) corresponds to a higher binding affinity for the PD-1 ligand. A high binding affinity corresponds to a greater intermolecular force between the ligand and the protein. A low binding affinity corresponds to a lower intermolecular force between the ligand and the protein. In some cases, an increase in ligand binding affinity can be represented as a decrease of the off-rate by, for example, at least 10-fold, at least 20-fold, at least 50-fold, at least 100-fold, at least 200-fold, at least 500-fold, or more.

The ability of an sPD-1 variant to bind to PD-L1 and/or PD-L2 can be determined, for example, by the ability of the putative ligand to bind to PD-L1 and/or PD-L2 coated on an assay plate. In one embodiment, the binding activity of sPD-1 variants to PD-L1 and/or PD-L2 can be assayed by either immobilizing the ligand, e.g., PD-L1 and/or PD-L2 or the sPD-1 variant. For example, the assay can include immobilizing PD-L1 and/or PD-L2 fused to a His-tag onto Ni-activated NTA resin beads. Agents can be added in an appropriate buffer and the beads incubated for a period of time at a given temperature. After washes to remove unbound material, the bound protein can be released with, for example, SDS, buffers with a high pH, and the like and analyzed.

Alternatively, binding affinity of an sPD-1 variant for PD-L1 and/or PD-L2 can be determined by displaying the sPD-1 variant on a microbial cell surface, e.g., a yeast cell surface and detecting the bound complex by, for example, flow cytometry. The binding affinity of sPD-1 for PD-1 ligands can be measured using any known method recognized in the art including, but not limited to, the method described in Examples, radioactive ligand binding assays, non-radioactive (fluorescent) ligand binding assays, surface plasmon resonance (SPR), such as Biacore™, Octet™, plasmon-waveguide resonance (PWR), thermodynamic binding assays, whole-cell ligand-binding assays, and structure-based ligand binding assays.

“Specific binding” or “specifically binds to” or is “specific for” a particular ligand or variant thereof means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target. In some embodiments, the binding affinity is measured using assays in the art as discussed above, such as a standard Biacore assay.

Specific binding for a particular ligand or variant thereof can be exhibited, for example, by a protein having a KD for another ligand protein of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular protein-ligand interaction. Typically, a protein that specifically binds a ligand will have a KD that is 20-, 50-, 100-, 200-, 500-, 1000-, 5,000-, 10,000- or more times greater for a control molecule relative to the protein.

By “residue” as used herein is meant a position in a protein and its associated amino acid identity. For example, Asparagine 297 (also referred to as Asn297 or N297) is a residue at position 297 in the human antibody IgG1.

By “interleukin-15” “IL-15” “IL15” or “MGC9721” as used herein refers to a mammalian interleukin 15 (preferably a primate interleukin 15, and more preferably a human interleukin 15) or a functional/biologically active fragment or variant thereof. The term “functional” or “biologically active” as disclosed herein refers to that a polypeptide of IL-15 fragment, or variant thereof has functionality similar (75% or greater) to that of a native IL-15 protein in at least one functional assay described below. Said IL-15 polypeptide can have at least 90%, 91%, 92%, 93%, 94%, 95%, 96%, 97%, 98% or 99% sequence identity to human IL-15 amino acid sequence (SEQ ID NO:10). In some cases, “IL-15” refers to a nucleotide encoding such an IL-15 polypeptide or a functional/biologically active fragment, or variant thereof. Functionally, IL-15 is a cytokine that regulates T cell and NK cell activation and proliferation. IL-15 and IL-2 share many biological activities, including binding to CD122, the IL-2$/IL-15$ receptor subunit. The number of CD8+ memory cells is controlled by a balance between this IL-15 and IL-2. IL-15 induces the activation of JAK kinases, as well as the phosphorylation and activation of transcription activators STAT3, STATS, and STAT6. IL-15 also increases the expression of apoptosis inhibitor BCL2L1/BCL-x(L). Exemplified functional assays of an IL-15 polypeptide include proliferation of T-cells (see, for example, Montes, et al., Clin Exp Immunol (2005) 142:292), proliferation induction on kit225 cell line (HORI et al., Blood, vol. 70(4), p:1069-72, 1987), and activation of NK cells, macrophages and neutrophils. Methods for isolation of particular immune cell subpopulations and detection of proliferation (i.e., 3H-thymidine incorporation) are well known in the art. Cell-mediated cellular cytotoxicity assays can be used to measure NK cell, macrophage and neutrophil activation. Cell-mediated cellular cytotoxicity assays, including release of isotopes (51Cr), dyes (e.g., tetrazolium, neutral red) or enzymes, are also well known in the art, with commercially available kits (Oxford Biomedical Research, Oxford, M; Cambrex, Walkersville, Md.; Invitrogen, Carlsbad, Calif.). IL-15 has also been shown to inhibit Fas mediated apoptosis (see, Demirci and Li, Cell Mol Immunol (2004) 1:123). Apoptosis assays, including for example, TUNEL assays and annexin V assays, are well known in the art with commercially available kits (R&D Systems, Minneapolis, Minn.). See also, Coligan, et al., Current Methods in Immunology, 1991-2006, John Wiley & Sons (US10894816B2, hereby entirely incorporated by reference).

The term “interleukin-15 receptor alpha” or “IL-15Rα” refers to a polypeptide that has at least 90%, 91%, 92%, 93%, 94%, 95%, 96%, 97%, 98% or 99% sequence identity to a native mammalian IL-15Rα amino acid sequence, or a nucleotide encoding such a biologically active polypeptide, meaning that such a polypeptide has functionality similar (75% or greater) to that of a native IL-15Rα protein in at least one functional assay. IL-15Rα is a cytokine receptor that specifically binds IL-15 with high affinity. One functional assay is specific binding to a native IL-15 protein.

“IL-15Rα sushi domain” as used herein refers to the sushi domain of IL-15Rα, which is based on a β-sandwich structure with one face containing three P-strands hydrogen-bonded to form a triple-stranded central region and the opposite face formed by two separate $-strands. IL-15Rα sushi domain is essential for interacting with IL-15 and mediating the biological function of IL-15, for example, it is crucial for the neutralization of IL-15-mediated T cell proliferation and rescue of apoptosis and necrosis. In some embodiments, IL-15Rα sushi domain refers to the ECD of human wild-type IL-15Rα sushi polypeptide as set forth in SEQ ID NO:11.

By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG, the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain. As outlined herein, in some cases, Fc domains inclusive of the hinge are used, with the hinge generally being used as a flexible linker. (Additionally, as further described herein, additional flexible linker components can be used either with or without the hinge).

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. Thus Fc refers to the last two constant region immunoglobulin domains of IgA, IgD, and IgG, the last three constant region immunoglobulin domains of IgE and IgM, and in some cases, includes the flexible hinge N-terminal to these domains. For IgA and IgM, Fc may include the J chain. For IgG, the Fc domain comprises immunoglobulin domains Cγ2 and Cγ3 and in some cases, includes the lower hinge region between Cγ1 and Cγ2. An “Fc fragment” in this context may contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.) Human IgG Fc domains are of particular use in the present disclosure and can be the Fc domain from human IgG1, IgG2, IgG3 or IgG4. In general, IgG1, IgG2 and IgG4 are used more frequently than IgG3. In some embodiments, amino acid modifications are made to the Fc region, for example to alter binding to one or more FcγR receptors or to the FcRn receptor, and/or to increase the half-life in vivo.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, an F296Y substitution in IgG2 is considered an IgG subclass modification. Similarly, because IgG1 has a proline at position 241 and IgG4 has a serine there, an IgG4 molecule with a S241P is considered an IgG subclass modification. Note that subclass modifications are considered amino acid substitutions herein.

By “non-naturally occurring modification” as used herein is meant an amino acid modification that is not isotypic. For example, because none of the IgGs comprise an asparagine at position 297, the substitution N297A in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include, but are not limited to, antibody-dependent cellular cytotoxicity (ADCC), antibody-dependent cellular phagocytosis (ADCP), and complement-dependent cytotoxicity (CDC). In many cases, it is desirable to ablate most or all effector functions using either different IgG isotypes (e.g., IgG4) or amino acid substitutions in the Fc domain; however, preserving binding to the FcRn receptor is desirable, as this contributes to the half-life of the fusion protein in human serum.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, hereby entirely incorporated by reference), as well as any undiscovered human FcγRs or FcγR isoforms or allotypes. An FcγR may be from any organism, including, but not limited to, humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene.

By “linker”, “domain linker”, “linker domain” or “linker peptide” as used herein have a length that is adequate to link two molecules in such a way that they assume the correct conformation relative to one another so that they retain the desired activity. The linker or linker peptide may predominantly include the following amino acid residues: Gly, Ser, Leu, or Gln. In one embodiment, the linker is from about 1 to 50 amino acids in length, preferably about 1 to 20 amino acids in length. In one embodiment, linkers of 1 to 20 amino acids in length may be used, with from about 8 to about 20 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including, but not limited to, polyethylene glycol (PEG), polypropylene glycol, polyoxyalkylenes, or copolymers of polyethylene glycol and polypropylene glycol, may find use as linkers.

In some embodiments, the linker is a “domain linker”, used to link any two domains as outlined herein together, such as to link the sPD-1 variant domain with (variant) Fc domain, or link the IL-15 domain or the IL-15Rα sushi domain with (variant) Fc domain. While any suitable linker can be used, many embodiments utilize a glycine-serine polymer, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some embodiments, the linker has the sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20 and SEQ ID NO:21.

By “target cell” as used herein is meant a cell that expresses a target polypeptide or protein.

By “host cell” in the context of producing the bispecific Fc fusion proteins comprising sPD-1 variant domain, IL-15 domain and IL-15Rα domain according to the disclosure herein is meant a cell that contains the exogenous nucleic acids encoding the components of the bispecific Fc fusion protein as disclosed herein and is capable of expressing such bispecific Fc fusion protein under suitable conditions. Suitable host cells are described below.

By “improved activity” or “improved function” herein is meant a desirable change of at least one biochemical property. An improved function in this context can be measured as a percentage increase or decrease of a particular activity, or as a “fold” change, with increases of desirable properties (e.g., increased binding affinity for PD-L1 and/or PD-L2, enhanced agonist activity of IL-15, extended half-life, and synergistic efficacy for treating cancer and/or infections, etc.). In general, percentage changes are used to describe changes in biochemical activity of less than 100%, and fold-changes are used to describe changes in biochemical activity of greater than 100% (as compared to the parent protein). In the present disclosure, percentage changes (usually increases) of biochemical activity of at least about 10%, 20%, 30%, 40%, 50%, 60%, 70%, 80%, 90%, 95%, 98% and 99% can be accomplished. In the present disclosure, a “fold increase” (or decrease) is measured as compared to the parent protein. In many embodiments, the improvement is at least one-and-a-tenth fold (1.1), one-and-a-half fold (1.5 fold), 2 fold, 3 fold, 4 fold, 5 fold, 6 fold, 7 fold, 8 fold, 9 fold, 10 fold, 50 fold, 100 fold, 200 fold or higher.

### C. Bispecific Fusion Proteins

As described herein, the bispecific Fc fusion proteins of the disclosure comprise an IL-15 domain, IL-15Rα sushi domain, an Fc domain, a soluble PD-1 (sPD-1) variant domain, and optionally domain linkers linking between those domains as needed.

As described herein, the format of the fusion protein can take on several configurations, with the component domains switching order in the protein (from N- to C-terminus).

In one embodiment, the bispecific Fc fusion protein comprises, from N- to C-terminus: a) the IL-15Rα sushi domain; b) the first domain linker; c) the IL-15 domain; d) the second domain linker; e) the Fc domain; f) the third domain linker; and g) the sPD-1 variant domain.

In a further embodiment, the bispecific Fc fusion protein comprises, from N- to C-terminus: a) the IL-15 domain; b) the first domain linker; c) the IL-15Rα sushi domain; d) the second domain linker; e) the Fc domain; f) the third domain linker; and g) the sPD-1 variant domain.

In an additional embodiment, the bispecific Fc fusion protein comprises, from N- to C-terminus: a) the sPD-1 variant domain; b) the first domain linker; c) the Fc domain; d) the second domain linker; e) the IL-15 domain; f) the third domain linker; and g) the IL-15Rα sushi domain.

In a further embodiment, the bispecific Fc fusion protein comprises, from N- to C-terminus: a) the sPD-1 variant domain; b) the first domain linker; c) the Fc domain; d) the second domain linker; e) the IL-15Rα sushi domain; f) the third domain linker; and g) the IL-15 domain.

In an additional embodiment, the bispecific Fc fusion protein comprises, from N- to C-terminus: a) the IL-15Rα sushi domain; b) the first domain linker; c) the IL-15 domain; d) the second domain linker; e) the sPD-1 variant domain; f) the third domain linker; and g) the Fc domain.

In a further embodiment, the bispecific Fc fusion protein comprises, from N- to C-terminus: a) the IL-15 domain; b) the first domain linker; c) the IL-15Rα sushi domain; d) the second domain linker; e) the sPD-1 variant domain; f) the third domain linker; and g) the Fc domain.

In an additional embodiment, the bispecific Fc fusion protein comprises, from N- to C-terminus: a) the sPD-1 variant domain; b) the first domain linker; c) the IL-15 domain; d) the second domain linker; e) the IL-15Rα sushi domain; f) the third domain linker; and g) the Fc domain.

In a further embodiment, the bispecific Fc fusion protein comprises, from N- to C-terminus: a) the sPD-1 variant domain; b) the first domain linker; c) the IL-15Rα sushi domain; d) the second domain linker; e) the IL-15 domain; f) the third domain linker; and g) the Fc domain.

In an additional embodiment, the bispecific Fc fusion protein comprises, from N- to C-terminus: a) the Fc domain; b) the first domain linker; c) the IL-15 domain; d) the second domain linker; e) the IL-15Rα sushi domain; f) the third domain linker; and g) the sPD-1 variant domain.

In a further embodiment, the bispecific Fc fusion protein comprises, from N- to C-terminus: a) the Fc domain; b) the first domain linker; c) the IL-15Rα sushi domain; d) the second domain linker; e) the IL-15 domain; f) the third domain linker; and g) the sPD-1 variant domain.

In an additional embodiment, the bispecific Fc fusion protein comprises, from N- to C-terminus: a) the Fc domain; b) the first domain linker; c) the sPD-1 variant domain; d) the second domain linker; e) the IL-15 domain; f) the third domain linker; and g) the IL-15Rα sushi domain.

In a further embodiment, the bispecific Fc fusion protein comprises, from N- to C-terminus: a) the Fc domain; b) the first domain linker; c) the sPD-1 variant domain; d) the second domain linker; e) the IL-15Rα sushi domain; f) the third domain linker; and g) the IL-15 domain.

In some embodiments, a linker is not used in linking the Fc domain with other domain(s). Note that in some cases, the same Fc fusion protein can be labeled somewhat differently.

For example, in the case where the Fc domain includes a hinge domain, the Fc fusion protein comprising sPD-1 variant domain-Fc domain or IL-15-Fc domain, or IL-15Rα-Fc domain still include a linker in the form of the hinge domain. Alternatively, this same protein may not have the hinge domain included in the Fc domain, in which case the fusion protein comprises sPD-1 variant domain-CH2-CH3, IL-15-CH2-CH3, or IL-15Rα-CH2-CH3.

Thus, in some embodiments, the present disclosure provides a bispecific Fc fusion protein as described herein, where the Fc domain comprises a hinge domain and the Fc domain is linked with other domains (i.e., sPD-1 variant domain, and/or IL-15 domain or IL-15Rα sushi domain) by the hinge domain in the format of (from N-terminus to C-terminus, or from C-terminus to N-terminus): other domain(s)-hinge domain-CH2-CH3.

In some embodiments, the present disclosure provides a bispecific Fc fusion protein as described above, where the Fc domain comprises a hinge domain and the Fc domain is linked with other domains (i.e., sPD-1 variant domain, and/or IL-15 domain or IL-15Rα sushi domain) by an additional linker in the format of (from N-terminus to C-terminus, or from C-terminus to N-terminus): other domain(s)-domain linker-hinge domain-CH2-CH3; other domain(s)-domain linker-CH2-CH3; other domain(s)-domain linker-CH2-CH3-hinge domain; or other domain(s)-domain linker-CH2− CH3.

In some embodiments, the present disclosure provides a bispecific Fc fusion protein as described above, where the Fc domain does not comprise a hinge domain and the Fc domain is linked with other domains (i.e., sPD-1 variant domain, and/or IL-15 domain or IL-15Rα sushi domain) by a domain linker (e.g., non-hinge) as described herein.

Table 1 below shows the amino acid sequences and DNA sequences of the present disclosure and their assigned SEQ ID NOs.

| | |
|---|---|
| ECD of human PD-1 without the first four amino acids | SEQ ID NO: 1 |
| sPD-1 variant 1: S38G/S63G/P65L/N92S/G100S/S103V/A108I/A116V | SEQ ID NO: 2 |
| sPD-1 variant 2: S38G/S63G/P65L/G100S/S103V/A108I/A116V | SEQ ID NO: 3 |
| sPD-1 variant 3: P65L/G100S/S103V/A108I/A116V | SEQ ID NO: 4 |
| sPD-1 variant 4: S63G/G100S/S103V/A108I/A116V | SEQ ID NO: 5 |
| sPD-1 variant 5: S63G/P65L/G100S/S103V/A108I/A116V | SEQ ID NO: 6 |
| sPD-1 variant 6: G100S/S103V/A108I/A116V | SEQ ID NO: 7 |
| sPD-1 variant 7: G100S/S103V/A108I | SEQ ID NO: 8 |
| sPD-1 variant 8: N92S/G100S/S103V/A108I/A116V | SEQ ID NO: 9 |
| Human IL-15 domain | SEQ ID NO: 10 |
| Human IL-15R alpha sushi domain | SEQ ID NO: 11 |
| Linker | SEQ ID NO: 12 |
| Linker variant 1 | SEQ ID NO: 13 |
| Linker variant 2 | SEQ ID NO: 14 |
| Linker variant 3 | SEQ ID NO: 15 |
| Linker variant 4 | SEQ ID NO: 16 |
| Linker variant 5 | SEQ ID NO: 17 |
| Linker variant 6 | SEQ ID NO: 18 |
| GS Linker | SEQ ID NO: 19 |
| GS Linker X2 | SEQ ID NO: 20 |
| GS Linker X3 | SEQ ID NO: 21 |
| Signal Peptide 1 | SEQ ID NO: 22 |
| Signal Peptide 2 | SEQ ID NO: 23 |
| Human IgG4 | SEQ ID NO: 24 |
| Variant Human IgG4 | SEQ ID NO: 25 |
| Fusion Protein 1 with GS linker variant 1 and GS linker X2 | SEQ ID NO: 26 |
| Fusion Protein 2 with GS linker variant 2 and GS linker X2 | SEQ ID NO: 27 |
| Fusion Protein 3 with GS linker variant 3 and GS linker X2 | SEQ ID NO: 28 |
| Fusion Protein 4 with GS linker variant 4 and GS linker X2 | SEQ ID NO: 29 |
| Fusion Protein 5 with GS linker variant 5 and GS linker X2 | SEQ ID NO: 30 |
| Fusion Protein 6 with GS linker variant 6 and GS linker X2 | SEQ ID NO: 31 |
| Fusion Protein 7 with GS linker variant 1 and GS linker X3 | SEQ ID NO: 32 |
| Fusion Protein 8 with GS linker variant 2 and GS linker X3 | SEQ ID NO: 33 |
| Fusion Protein 9 with GS linker variant 3 and GS linker X3 | SEQ ID NO: 34 |
| Fusion Protein 10 with GS linker variant 4 and GS linker X3 | SEQ ID NO: 35 |
| Fusion Protein 11 with GS linker variant 5 and GS linker X3 | SEQ ID NO: 36 |
| Fusion Protein 12 with GS linker variant 6 and GS linker X3 | SEQ ID NO: 37 |
| Preprotein 1 including Signal Peptide 1 and Fusion protein 1 | SEQ ID NO: 38 |
| Preprotein 2 including Signal Peptide 1 and Fusion protein 2 | SEQ ID NO: 39 |
| Preprotein 3 including Signal Peptide 1 and Fusion protein 3 | SEQ ID NO: 40 |
| Preprotein 4 including Signal Peptide 1 and Fusion protein 4 | SEQ ID NO: 41 |
| Preprotein 5 including Signal Peptide 1 and Fusion protein 5 | SEQ ID NO: 42 |
| Preprotein 6 including Signal Peptide 1 and Fusion protein 6 | SEQ ID NO: 43 |
| Preprotein 7 including Signal Peptide 1 and Fusion protein 7 | SEQ ID NO: 44 |
| Preprotein 8 including Signal Peptide 1 and Fusion protein 8 | SEQ ID NO: 45 |
| Preprotein 9 including Signal Peptide 1 and Fusion protein 9 | SEQ ID NO: 46 |
| Preprotein 10 including Signal Peptide 1 and Fusion protein 10 | SEQ ID NO: 47 |
| Preprotein 11 including Signal Peptide 1 and Fusion protein 11 | SEQ ID NO: 48 |
| Preprotein 12 including Signal Peptide 1 and Fusion protein 12 | SEQ ID NO: 49 |
| Preprotein 13 including Signal Peptide 2 and Fusion protein 1 | SEQ ID NO: 50 |
| Preprotein 14 including Signal Peptide 2 and Fusion protein 2 | SEQ ID NO: 51 |
| Preprotein 15 including Signal Peptide 2 and Fusion protein 3 | SEQ ID NO: 52 |
| Preprotein 16 including Signal Peptide 2 and Fusion protein 4 | SEQ ID NO: 53 |
| Preprotein 17 including Signal Peptide 2 and Fusion protein 5 | SEQ ID NO: 54 |
| Preprotein 18 including Signal Peptide 2 and Fusion protein 6 | SEQ ID NO: 55 |
| Preprotein 19 including Signal Peptide 2 and Fusion protein 7 | SEQ ID NO: 56 |
| Preprotein 20 including Signal Peptide 2 and Fusion protein 8 | SEQ ID NO: 57 |
| Preprotein 21 including Signal Peptide 2 and Fusion protein 9 | SEQ ID NO: 58 |
| Preprotein 22 including Signal Peptide 2 and Fusion protein 10 | SEQ ID NO: 59 |
| Preprotein 23 including Signal Peptide 2 and Fusion protein 11 | SEQ ID NO: 60 |
| Preprotein 24 including Signal Peptide 2 and Fusion protein 12 | SEQ ID NO: 61 |
| Fusion Protein 6A | SEQ ID NO: 62 |
| Fusion Protein 6B | SEQ ID NO: 63 |
| Fusion Protein 6C | SEQ ID NO: 64 |
| Fusion Protein 6D | SEQ ID NO: 65 |
| Fusion Protein 6E | SEQ ID NO: 66 |
| Fusion Protein 6F | SEQ ID NO: 67 |
| Fusion Protein 6G | SEQ ID NO: 68 |
| Fusion Protein 6H | SEQ ID NO: 69 |
| Fusion Protein 6I | SEQ ID NO: 70 |
| Fusion Protein 6J | SEQ ID NO: 71 |
| Fusion Protein 6K | SEQ ID NO: 72 |
| Preprotein 18A | SEQ ID NO: 73 |
| Preprotein 18B | SEQ ID NO: 74 |
| Preprotein 18C | SEQ ID NO: 75 |
| Preprotein 18D | SEQ ID NO: 76 |
| Preprotein 18E | SEQ ID NO: 77 |
| Preprotein 18F | SEQ ID NO: 78 |
| Preprotein 18G | SEQ ID NO: 79 |
| Preprotein 18H | SEQ ID NO: 80 |
| Preprotein 18I | SEQ ID NO: 81 |
| Preprotein 18J | SEQ ID NO: 82 |
| Preprotein 18K | SEQ ID NO: 83 |
| DNA sequence encoding preprotein 1 | SEQ ID NO: 84 |
| DNA sequence encoding preprotein 2 | SEQ ID NO: 85 |
| DNA sequence encoding preprotein 3 | SEQ ID NO: 86 |
| DNA sequence encoding preprotein 7 | SEQ ID NO: 87 |
| DNA sequence encoding preprotein 8 | SEQ ID NO: 88 |
| DNA sequence encoding preprotein 9 | SEQ ID NO: 89 |
| DNA sequence encoding preprotein 13 | SEQ ID NO: 90 |
| DNA sequence encoding preprotein 14 | SEQ ID NO: 91 |
| DNA sequence encoding preprotein 15 | SEQ ID NO: 92 |
| DNA sequence encoding preprotein 19 | SEQ ID NO: 93 |
| DNA sequence encoding preprotein 20 | SEQ ID NO: 94 |
| DNA sequence encoding preprotein 21 | SEQ ID NO: 95 |
| Amino acid sequence of ECD of human PD-1 | SEQ ID NO: 96 |

1. sPD-1 Variant Domain

The sPD-1 variant domain of the present disclosure comprises the soluble ECD of human PD-1 with variants. The sPD-1 variants serve to increase the binding affinity and/or specificity for PD-L1 and/or PD-L2 compared to the wild-type PD-1 as determined by the binding affinity assays in the art, such as a Biacore assay, or by an in-house developed, ELISA-based Bioassay as disclosed in Example 2.

In some embodiments, the sPD-1 variants of the present disclosure are antagonists that bind to and block a PD-1 ligand (e.g., PD-L1 and/or PD-L2) and thereby interfere with or inhibit the binding of the ligand to its receptor PD-1. The antagonists can enhance an immune response by inhibiting the signal transduction pathway mediated by PD-1 via reducing the amount of ligand available to bind the PD-1 receptor. As such, a more robust immune response can be produced by the subject.

In some cases, a useful sPD-1 variant domain specifically binds to PD-L1 and/or PD-L2 on a target cell, e.g., on a cancer cell and thereby reduces (e.g., blocks, prevents, etc.) the interaction between the PD-L1/PD-L2 and PD-1 (e.g., wild-type PD-1 on an immune cell, e.g., on a T cell). Thus, an sPD-1 variant provided herein can act as an engineered decoy receptor for PD-L1 and/or PD-L2. By reducing the interaction between PD-L1 and/or PD-L2 and wild-type PD-1, the sPD-1 variant domain can decrease the immune inhibitory signals produced by the PD-L/PD-1 interaction, and therefore can increase the immune response (e.g., by increasing T cell activation). A suitable sPD-1 variant domain can comprise the portion of PD-1 that is sufficient to bind PD-1 ligand at a recognizable affinity, e.g., high affinity, which normally lies between the signal sequence and the transmembrane domain, or a fragment thereof that retains the binding activity.

In some embodiments, the sPD-1 variants include amino acid substitutions, deletions or insertions or any combination thereof to the WT PD-1 domain as set forth in SEQ ID NO:96 that increases or enhances its binding activity to either PD-L1, PD-L2 or both as compared to wild-type PD-1.

In some embodiments, the sPD-1 variants include amino acid substitutions, deletions or insertions or any combination thereof to the WT PD-1 fragment as set forth in SEQ ID NO:1 that increases or enhances its binding activity to either PD-L1, PD-L2 or both as compared to wild-type PD-1.

The present disclosure provides sPD-1 variant domains comprising at least one amino acid substitution at one or more (e.g., several) positions corresponding to positions 38, 63, 65, 92, 100, 103, 108 and 116 as compared to the human wild-type parent PD-1 fragment of SEQ ID NO:1, using the numbering starting from the mature region. In some embodiments, the sPD-1 variant has at least 80, 81, 82, 83, 84, 85, 86, 87, 88, 89, 90, 91, 92, 93, 94, 95, 96, 97, 98 or 99%, but less than 100, 99, 98, 97, 96, 95, 94, 93, 92, 91 or 90% sequence identity to the parent PD-1 domain. In some embodiments, the parent PD-1 domain is SEQ ID NO:1. In a preferred embodiment, the sPD-1 variant domain has at least 95%, at least 96%, at least 97%, at least 98%, or at least 99%, but less than 100% sequence identity to SEQ ID NO:1. In some embodiments, as noted herein, the sPD-1 variant domain can have N-terminal and/or C-terminal truncations compared to wild-type sPD-1 as set forth in SEQ ID NO:96 as long as the truncated variant sPD-1 retains biological activity (e.g., binding to PD-L1 and/or PD-L2). To be clear, the sPD-1 variants of the present disclosure are not naturally occurring and have at least one amino acid substitution as compared to the wild-type sPD-1 and thus do not have SEQ ID NO:1 or SEQ ID NO:96.

The present disclosure provides sPD-1 variant domains comprising one or more amino acid substitutions at one or more (e.g., several) positions corresponding to positions selected from the group consisting of positions 38, 63, 65, 92, 100, 103, 108 and 116 of SEQ ID NO: 1.

In some embodiment, the sPD-1 variant domains as described herein comprise at least one amino acid substitution at a position corresponding to position 38 of SEQ ID NO: 1. In some embodiment, the sPD-1 variant domains as described herein comprise at least one amino acid substitution at a position corresponding to position 63 of SEQ ID NO: 1. In some embodiment, the sPD-1 variant domains as described herein comprise at least one amino acid substitution at a position corresponding to position 65 of SEQ ID NO: 1. In some embodiment, the sPD-1 variant domains as described herein comprise at least one amino acid substitution at a position corresponding to position 92 of SEQ ID NO: 1. In some embodiment, the sPD-1 variant domains as described herein comprise at least one amino acid substitution at a position corresponding to position 100 of SEQ ID NO: 1. In some embodiment, the sPD-1 variant domains as described herein comprise at least one amino acid substitution at a position corresponding to position 103 of SEQ ID NO: 1. In some embodiment, the sPD-1 variant domains as described herein comprise at least one amino acid substitution at a position corresponding to position 108 of SEQ ID NO: 1. In some embodiment, the sPD-1 variant domains as described herein comprise at least one amino acid substitution at a position corresponding to position 116 of SEQ ID NO: 1.

In some embodiments, the sPD-1 variant domain as described herein has amino acid substitution(s) at one of said positions, two of said positions, three of said positions, four of said positions, five of said positions, six of said positions, seven of said positions or eight of said positions.

In some embodiments, the present disclosure provides sPD-1 variant domains comprising one or more of amino acid substitution(s) selected from the group consisting of: S38G, S63G, P65L, N92S, G100S, S103V, A1081, and A116V as compared to SEQ ID NO:1.

In some embodiments, the present disclosure provides sPD-1 variant domains comprising a set of amino acid substitutions selected from the group consisting of N92S/G100S/S103V/A108I/A116V, S38G/S63G/P65L/N92S/G100S/S103V/A108I/A116V, S38G/S63G/P65L/G100S/S103V/A108I/A116V, P65L/G100S/S103V/A108I/A116V, S63G/G100S/S103V/A108I/A116V, S63G/P65L/G100S/S103V/A108I/A116V, G100S/S103V/A1081/A116V and G100S/S103V/A108I as compared to SEQ ID NO:1.

In some embodiments, the present disclosure provides sPD-1 variant domains comprising a set of amino acid substitutions N92S/G100S/S103V/A1081/A116V as compared to SEQ ID NO:1.

In some embodiments, the present disclosure provides sPD-1 variant domains comprising a set of amino acid substitutions S38G/S63G/P65L/N92S/G100S/S103V/A108I/A116V as compared to SEQ ID NO:1.

In some embodiments, the present disclosure provides sPD-1 variant domains comprising a set of amino acid substitutions S38G/S63G/P65L/G100S/S103V/A108I/A116V as compared to SEQ ID NO:1.

In some embodiments, the present disclosure provides sPD-1 variant domains comprising a set of amino acid substitutions P65L/G100S/S103V/A108I/A116V as compared to SEQ ID NO:1.

In some embodiments, the present disclosure provides sPD-1 variant domains comprising a set of amino acid substitutions S63G/G100S/S103V/A108I/A116V as compared to SEQ ID NO:1.

In some embodiments, the present disclosure provides sPD-1 variant domains comprising a set of amino acid substitutions S63G/P65L/G100S/S103V/A108I/A116V as compared to SEQ ID NO:1.

In some embodiments, the present disclosure provides sPD-1 variant domains comprising a set of amino acid substitutions G100S/S103V/A108I/A116V as compared to SEQ ID NO:1.

In some embodiments, the present disclosure provides sPD-1 variant domains comprising a set of amino acid substitutions G100S/S103V/A108I as compared to SEQ ID NO:1.

In some embodiments, the present disclosure provides sPD-1 variant domains comprising an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8 and SEQ ID NO:9.

In some embodiments, the present disclosure provides sPD-1 variant domains comprising the amino acid sequence of SEQ ID NO:2. In some embodiments, the present disclosure provides sPD-1 variant domains comprising the amino acid sequence of SEQ ID NO:3. In some embodiments, the present disclosure provides sPD-1 variant domains comprising the amino acid sequence of SEQ ID NO:4. In some embodiments, the present disclosure provides sPD-1 variant domains comprising the amino acid sequence of SEQ ID NO:5. In some embodiments, the present disclosure provides sPD-1 variant domains comprising the amino acid sequence of SEQ ID NO:6. In some embodiments, the present disclosure provides sPD-1 variant domains comprising the amino acid sequence of SEQ ID NO:7. In some embodiments, the present disclosure provides sPD-1 variant domains comprising the amino acid sequence of SEQ ID NO:8. In some embodiments, the present disclosure provides sPD-1 variant domains comprising the amino acid sequence of SEQ ID NO:9.

In some embodiments, the sPD-1 variant domain comprises an amino acid substitution of the serine at a position corresponding to the position 38 of SEQ ID NO:1. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is S38G.

In some embodiments, the sPD-1 variant domain comprises an amino acid substitution of the serine at a position corresponding to the position 63 of SEQ ID NO:1. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is S63G.

In some embodiments, the sPD-1 variant domain comprises an amino acid substitution of the proline at a position corresponding to the position 65 of SEQ ID NO: 1. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, leucine, methionine, phenylalanine, tryptophan, valine and tyrosine, with some embodiments not utilizing cysteine (due to possible disulfide formation). In some embodiments, the amino acid substitution is P65L.

In some embodiments, the sPD-1 variant domain comprises an amino acid substitution of the asparagine at a position corresponding to the position 92 of SEQ ID NO:1. In some embodiments, the substitution is with any other of the 19 naturally occurring amino acids, serine, threonine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is N92S.

In some embodiments, the sPD-1 variant domain comprises an amino acid substitution of the glycine at a position corresponding to the position 100 of SEQ ID NO:1. In some embodiments, the substitution is with any other of the 19 naturally occurring amino acids, serine, threonine, asparagine, glutamic acid, glutamine, aspartic acid, lysine, arginine, histidine, cysteine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is G100S.

In some embodiments, the sPD-1 variant domain comprises an amino acid substitution of the serine at a position corresponding to the position 103 of SEQ ID NO:1. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is S103V.

In some embodiments, the sPD-1 variant domain comprises an amino acid substitution of the alanine at a position corresponding to the position 108 of SEQ ID NO:1. In some embodiments, the substitution is with any other of the 19 naturally occurring amino acids, serine, threonine, asparagine, glutamic acid, glutamine, aspartic acid, lysine, arginine, histidine, cysteine, glycine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is A1081.

In some embodiments, the sPD-1 variant domain comprises an amino acid substitution of the alanine at a position corresponding to the position 116 of SEQ ID NO:1. In some embodiments, the substitution is with any other of the 19 naturally occurring amino acids, serine, threonine, asparagine, glutamic acid, glutamine, aspartic acid, lysine, arginine, histidine, cysteine, glycine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is A116V.

In some embodiments, the sPD-1 variant protein is shorter than the full length ECD of PD-1. In some embodiments, the sPD-1 variants may comprise a truncated version of the ECD, as long as the truncated form retains the ability to bind human PD-L1 and/or PD-L2 as measured by one of the binding assays outlined herein. As is known in the art, both N- and C-terminal truncations are possible, e.g., from about residue 1, 5, 10, 15, 20, 25, 30, to about residue 33, 35, 40, 45, or 50 of SEQ ID NO: 96. In some cases, only a few amino acids (e.g., 1, 2, 3, 4, 5 or 6) are removed from either or both of the N and C-terminus, as long as activity is retained.

In some embodiments, the sPD-1 variant described herein has a binding affinity for a PD-1 ligand (i.e., PD-L1 and/or PD-L2) that is better than the wild-type PD-1 polypeptide/domain. In some embodiments, the sPD-1 variants have a binding affinity for PD-L1 and/or PD-L2 that is at least 1-fold, 2-fold, 3-fold, 4-fold, 5-fold, 10-fold, 50-fold, 100-fold, 200-fold or greater than that of the wild-type PD-1. In some embodiments, the sPD-1 variants can have a binding affinity for PD-L1 that is at least 1-fold, 2-fold, 3-fold, 4-fold, 5-fold, 10-fold, 50-fold, 100-fold, 200-fold or greater than that of the wild-type PD-1. In some embodiments, the sPD-1 variants can have a binding affinity for PD-L2 that is at least 1-fold, 2-fold, 3-fold, 4-fold, 5-fold, 10-fold, 50-fold, 100-fold, 200-fold or greater than that of the wild-type PD-1.

In certain embodiments, the binding affinity of the sPD-1 variant for PD-L1, PD-L2 or both is increased by at least about 0.5%, 1%, 2%, 3%, 4%, 5%, 6%, 7%, 8%, 9%, 10%, 15%, 20%, 25%, 30%, 35%, 40%, 45%, 50% or higher as compared to that of the wild-type PD-1. In other embodiments, the sPD-1 variants of the present disclosure have a binding affinity of less than about 1×10⁻⁸ M, 1×10⁻⁹ M, 1×10⁻¹⁰ M or 1×10⁻¹² M for PD-L1 and/or PD-L2. The sPD-1 variants of the present disclosure can have a binding affinity of less than about 1×10⁻⁸ M, 1×10⁻⁹ M, 1×10⁻¹⁰ M or 1×10⁻¹² M for PD-L1. The sPD-1 variants of the present disclosure can have a binding affinity of less than about 1×10⁻⁸ M, 1×10⁹ M, 1×10⁻¹⁰ M or 1×10⁻¹² M for PD-L2. In yet other embodiments, the sPD-1 variants inhibit or compete with wild-type PD-1 binding to PD-L1 and/or PD-L2 either in vivo, in vitro or both.

In some embodiments, the sPD-1 variant has a dissociation half-life for PD-L1 and/or PD-L2 that is 2-fold or more (e.g., 5-fold or more, 10-fold or more, 100-fold or more, 500-fold or more, 1000-fold or more, 5000-fold or more, 10000-fold or more, etc.) greater than the dissociation half-life for PD-L1 of a wild-type PD-1.

In some embodiments, the present disclosure provides a composition comprising any one of the sPD-1 variant domains as disclosed herein. In some embodiments, the present disclosure provides a composition comprising any one of the sPD-1 variant domains as disclosed herein and any one of the Fc domains as disclosed herein. In some embodiments, the present disclosure provides a composition comprising any one of the sPD-1 variant domains as disclosed herein, any one of the Fc domains as disclosed herein, and any one of the domain linkers as disclosed herein.

In some embodiments, the present disclosure provides a composition comprising bispecific Fc fusion protein(s) comprising the sPD-1 variant domain as disclosed herein, an IL-15 domain, an IL-15Rα sushi domain, an Fc domain and optional domain linkers as disclosed herein.

### 2. Fc Domain

As discussed herein, in addition to the sPD-1 variant domain described above, the fusion proteins of the present disclosure also include an Fc domain of antibodies that generally are based on the IgG class, which has several subclasses, including, but not limited to, IgG1, IgG2, IgG3, and IgG4. As described herein, the Fc domain optionally includes the hinge domain of an IgG antibody.

Human IgG Fc domains are of particular use in the present disclosure, and can be the Fc domain from human IgG1, IgG2, IgG3 or IgG4. In general, IgG1, IgG2 and IgG4 are used more frequently than IgG3.

The Fc domain of a human IgG protein included in the fusion protein of the present disclosure confers a significant increase in half-life of the fusion protein, and provides additional binding or interaction with the Ig molecules. In some embodiments, the bispecific-Fc fusion protein can facilitate purification, multimerization, binding and neutralizing other molecules as compared to the corresponding fusion protein without the Fc domain.

The Fc domains that find use in the disclosure can also contain Fc variants to alter function as needed. However, any Fc variants generally need to retain both the ability to form dimers as well as the ability to bind FcRn. Thus, while many of the embodiments herein rely on the use of a human IgG4 domain so as to avoid effector function, Fc variants can be made that augment or abrogate function in other IgG domains. Thus, for example, ablation variants that reduce or eliminate effector function in IgG1 or IgG2 can be used, and/or Fc variants that confer tighter binding to the FcRn can be used, as will be appreciated by those in the art.

In some embodiments, the Fc domain of the present disclosure is a human IgG Fc domain or a variant human IgG Fc domain. In some embodiments, the Fc domain of the present disclosure is a human IgG Fc domain. In some embodiments, the Fc domain of the present disclosure is a variant human IgG Fc domain.

a. IgG4 Fc Domains

The IgG4 subclass is distinguished from the other IgG subclasses, as it exhibits negligible binding to the C1q protein complex and is unable to activate the classical complement pathway (A. Nirula et al., 2011, Current Opinion in Rheumatology 23:119-124, hereby entirely incorporated by reference). As a result, IgG4 finds use in the present disclosure as it has no significant effector function, and is thus used to block the receptor-ligand binding without cell depletion.

In another embodiment, the Fc domains of the present disclosure are human IgG4 Fc domains.

In some embodiments, the Fc domain of the present disclosure comprises the hinge-CH2-CH3 of human IgG4.

In some embodiments, the Fc domain of the present disclosure comprises the CH2-CH3 of human IgG4.

In another embodiment, the Fc domains of the present disclosure are variant human IgG4 Fc domains. However, the variant Fc domains herein still retain the ability to form a dimer with another Fc domain as measured using known, as well as the ability to bind to FcRn, as this contributes significantly to the increase in serum half life of the fusion proteins herein.

The variant IgG4 Fc domain can include an addition, deletion, substitution or any combination thereof compared with the parent human IgG4 Fc domain.

In some embodiments, the variant human IgG4 Fc domains of the present disclosure can have at least about 80%, 81%, 82%, 83%, 84%, 85%, 86%, 87%, 88%, 89%, 90%, 91%, 92%, 93%, 94%, 95%, 96%, 97%, 98% or 99% but less than 100% identity to the corresponding parental human IgG4 Fc domain (using the identity algorithms discussed above, with one embodiment utilizing the BLAST algorithm as is known in the art, using default parameters).

In some embodiments, the variant human IgG4 Fc domains of the present disclosure can have from 1, 2, 3, 4, 5, 6, 7, 8, 9, 10, 11,12, 13, 14, 15, 16, 11, 12, 13, 14, 15, 16, 17, 18, 19 or 20 amino acid modifications as compared to the parental human IgG4 Fc domains as set forth in SEQ ID NO:24.

In some embodiments, the variant human IgG4 Fc domain comprises an amino acid substitution of the serine at position 228 to proline according to the EU numbering index.

In some embodiments, the Fc domain of the present disclosure comprises the amino acid sequence of SEQ ID NO:24.

In some embodiments, the Fc domain of the present disclosure comprises the amino acid sequence of SEQ ID NO:25.

b. Other IgG Fc Domains

In some embodiments, the Fc domains of the present disclosure can be the Fc domains from other IgGs than IgG4, such as human IgG1, IgG2 or IgG3. In general, IgG1 and IgG2 are used more frequently than IgG3.

In some embodiments, the Fc domain of the present disclosure is the Fc domain of human IgG1.

In some embodiments, the Fc domain of the present disclosure is the Fc domain of human IgG2.

In some embodiments, the Fc domain of the present disclosure is a variant human IgG1 Fc domain.

In some embodiments, the Fc domain of the present disclosure is a variant human IgG2 Fc domain.

In some embodiments, the variant human IgG1 Fc domains of the disclosure can have at least about 80%, 81%, 82%, 83%, 84%, 85%, 86%, 87%, 88%, 89%, 90%, 91%, 92%, 93%, 94%, 95%, 96%, 97%, 98% or 99% but less than 100% identity to the corresponding parental human IgG1 Fc domain (using the identity algorithms discussed above, with one embodiment utilizing the BLAST algorithm as is known in the art, using default parameters).

In some embodiments, the variant human IgG2 Fc domains of the disclosure can have at least about 80%, 81%, 82%, 83%, 84%, 85%, 86%, 87%, 88%, 89%, 90%, 91%, 92%, 93%, 94%, 95%, 96%, 97%, 98% or 99% but less than 100% identity to the corresponding parental human IgG2 Fc domain (using the identity algorithms discussed above, with one embodiment utilizing the BLAST algorithm as is known in the art, using default parameters).

In some embodiments, the variant human IgG3 Fc domains of the disclosure can have at least about 80%, 81%, 82%, 83%, 84%, 85%, 86%, 87%, 88%, 89%, 90%, 91%, 92%, 93%, 94%, 95%, 96%, 97%, 98% or 99% but less than 100% identity to the corresponding parental human IgG3 Fc domain (using the identity algorithms discussed above, with one embodiment utilizing the BLAST algorithm as is known in the art, using default parameters).

In some embodiments, the variant human IgG1 Fc domains of the disclosure can have from 1, 2, 3, 4, 5, 6, 7, 8, 9, 10, 11,12, 13, 14, 15, 16, 11, 12, 13, 14, 15, 16, 17, 18, 19 or 20 amino acid modifications as compared to the parental human IgG1 Fc domains.

In some embodiments, the variant human IgG2 Fc domains of the disclosure can have from 1, 2, 3, 4, 5, 6, 7, 8, 9, 10, 11,12, 13, 14, 15, 16, 11, 12, 13, 14, 15, 16, 17, 18, 19 or 20 amino acid modifications as compared to the parental human IgG2 Fc domains.

In some embodiments, the variant human IgG3 Fc domains of the disclosure can have from 1, 2, 3, 4, 5, 6, 7, 8, 9, 10, 11,12, 13, 14, 15, 16, 11, 12, 13, 14, 15, 16, 17, 18, 19 or 20 amino acid modifications as compared to the parental human IgG3 Fc domains.

### 3. IL-15 Domain

The IL-15 domain of the disclosure comprises a mammalian IL-15, or a biologically active fragment or variant thereof. In a preferred embodiment, the IL-15 domain is a primate IL-15, or a biologically active fragment or variant thereof. In a more preferred embodiment, the IL-15 domain is a human IL-15. The term “a biologically active fragment, or variant thereof” of IL-15 as disclosed herein refers to a polypeptide that has at least about 80%, 81%, 82%, 83%, 84%, 85%, 86%, 87%, 88%, 89%, 90%, 91%, 92%, 93%, 94%, 95%, 96%, 97%, 98% or 99% but less than 100% sequence identity to the wild-type IL-15, wherein the polypeptide has functionality similar (75% or greater) to that of the wild-type IL-15 in at least one functional assay described herein. Therefore, the term “a biologically active fragment, or variant thereof of human IL-15” as disclosed herein refers to a polypeptide that has at least about 80%, 81%, 82%, 83%, 84%, 85%, 86%, 87%, 88%, 89%, 90%, 91%, 92%, 93%, 94%, 95%, 96%, 97%, 98% or 99% but less than 100% sequence identity to human IL-15 as set forth in SEQ ID NO: 10, wherein the polypeptide has functionality similar (75% or greater) to that of human IL-15 protein in at least one functional assay described herein.

The IL-15 domain serves to stimulate the proliferation and activation of NK, Natural killer T (NKT) and CD8+ T cells, especially memory phenotype CD8+ T cells, leading to increased cytotoxicity and production of IFN-γ and IFN-α. In addition, the IL-15 domain inhibits apoptosis of immune cells by increasing expression of anti-apoptotic and decreasing production of pro-apoptotic proteins. Exemplified functional assays of an IL-15 polypeptide include proliferation of T-cells (see, for example, Montes, et al., Clin Exp Immunol (2005) 142:292), proliferation induction on kit225 cell line (HORI et al., Blood, vol. 70(4), p:1069-72, 1987), and activation of NK cells, macrophages and neutrophils. Cell-mediated cellular cytotoxicity assays can be used to measure NK cell, macrophage and neutrophil activation. Cell-mediated cellular cytotoxicity assays, including release of isotopes (51Cr), dyes (e.g., tetrazolium, neutral red) or enzymes, are also well known in the art, with commercially available kits (Oxford Biomedical Research, Oxford, M; Cambrex, Walkersville, Md.; Invitrogen, Carlsbad, Calif.). IL-15 has also been shown to inhibit Fas mediated apoptosis (see Demirci and Li, Cell Mol Immunol (2004) 1:123). Apoptosis assays, including for example, TUNEL assays and annexin V assays, are well known in the art with commercially available kits (R&D Systems, Minneapolis, Minn.). See also, Coligan, et al., Current Methods in Immunology, 1991-2006, John Wiley & Sons (U.S. Ser. No. 10/894,816B2, hereby entirely incorporated by reference). In most embodiments, the IL-15 binding activity of the present disclosure was analyzed using an in-house developed ELISA based Bioassay as disclosed in Example 2.

In some embodiments, the IL-15 domain is human IL-15. In some embodiments, the IL-15 domain comprises the amino acid sequence of SEQ ID NO:10.

In some embodiments, the IL-15 domain is a biologically active fragment or variant of human IL-15, and has at least 90%, 91%, 92%, 93%, 94%, 95%, 96%, 97%, 98% or 99% sequence identity to human IL-15 amino acid sequence. In some embodiments, the IL-15 domain is a biologically active fragment or variant of human IL-15, and has at least about 80%, 81%, 82%, 83%, 84%, 85%, 86%, 87%, 88%, 89%, 90%, 91%, 92%, 93%, 94%, 95%, 96%, 97%, 98% or 99% but less than 100% sequence identity to the amino acid sequence of SEQ ID NO:10. 4. IL-15Rα Sushi Domain

The IL-15Rα sushi domain of the present disclosure comprises the sushi domain of a mammalian IL-15Rα, or a biologically active fragment or variant thereof. In a preferred embodiment, the IL-15Rα sushi domain is a sushi domain of a primate IL-15Rα, or a biologically active fragment or variant thereof. In a more preferred embodiment, the IL-15Rα sushi domain is the sushi domain of the human IL-15Rα, or a biologically active fragment or variant thereof. The term “a biologically active fragment, or variant thereof” of IL-15Rα as disclosed herein refers to a polypeptide that has at least 80%, 85%, 90%, 91%, 92%, 93%, 94%, 95%, 96%, 97%, 98% or 99% sequence identity to the wild-type IL-15Rα, wherein the polypeptide has functionality similar (75% or greater) to that of the wild-type IL-15Rα in at least one functional assay described herein. Therefore, the term “a biologically active fragment, or variant thereof of human IL-15Rα” as disclosed herein refers to a polypeptide that has at least about 80%, 81%, 82%, 83%, 84%, 85%, 86%, 87%, 88%, 89%, 90%, 91%, 92%, 93%, 94%, 95%, 96%, 97%, 98% or 99% but less than 100% sequence identity to the ECD of human IL-15Rα as set forth in SEQ ID NO: 11, wherein the polypeptide has functionality similar (75% or greater) to that of human IL-15Rα in at least one functional assay described herein.

IL-15Rα is a cytokine receptor that specifically binds IL-15 with high affinity. The sushi domain of IL-15Rα is essential for interacting with IL-15 and mediating the biological function of IL-15, for example, it is crucial for the neutralization of IL-15-mediated T cell proliferation and rescue of apoptosis and necrosis. Its binding activity can be measured for specific binding to a native IL-15 protein using functional assays as known in the art, e.g., ELISA.

In some embodiments, the IL-15Rα sushi domain comprises the amino acid sequence of the sushi domain of human IL-15Rα. In some embodiments, the IL-15Rα sushi domain comprises the amino acid sequence of the ECD of human IL-15Rα sushi domain as set forth in SEQ ID NO:11.

In some embodiments, the IL-15Rα sushi domain is a biologically active fragment or variant of the sushi domain of human IL-15Rα, and has at least about 80%, 81%, 82%, 83%, 84%, 85%, 86%, 87%, 88%, 89%, 90%, 91%, 92%, 93%, 94%, 95%, 96%, 97%, 98% or 99% but less than 100% sequence identity to the amino acid sequence of the sushi domain of human IL-15Rα. In some embodiments, the IL-15 domain is a biologically active fragment or variant of the ECD of human IL-15Ra sushi domain as set forth in SEQ ID NO:11, and has at least about 80%, 81%, 82%, 83%, 84%, 85%, 86%, 87%, 88%, 89%, 90%, 91%, 92%, 93%, 94%, 95%, 96%, 97%, 98% or 99% but less than 100% sequence identity to the amino acid sequence of SEQ ID NO: 11.

### 5. Domain Linkers

The four domains described above (i.e., an sPD-1 variant domain, an IL-15 domain, an IL-15Rα sushi domain, and an Fc domain) are generally linked using domain linkers as described herein. In the context of the present disclosure, what is important is that sPD-1 variant domain and IL-15 domain/IL-15Rα sushi domain are attached to the Fc domain using a flexible linker in such a way that the three domains-sPD-1 variant, IL-15 (with IL-15Rα sushi domain) and Fc domain can act independently. This can be accomplished in a variety of ways, using traditional linkers and/or the hinge linker.

While any suitable linker can be used, the domain linker may predominantly include the following amino acid residues: Gly, Ser, Leu, or Gln. Useful linkers include glycine-serine polymers, including for example (GS)n, (GSGGS)n, (GGGGS)n, and (GGGS)n, where n is an integer of at least one (and generally from 3 to 5). In some embodiments, the linkers include glycine-alanine polymers, alanine-serine polymers, and other flexible linkers that allow for recombinant attachment of the two domains with sufficient length and flexibility to allow each domain to retain its biological function. In some embodiments, a variety of nonproteinaceous polymers, including, but not limited to, polyethylene glycol (PEG), polypropylene glycol, polyoxyalkylenes, or copolymers of polyethylene glycol and polypropylene glycol, may find use as linkers.

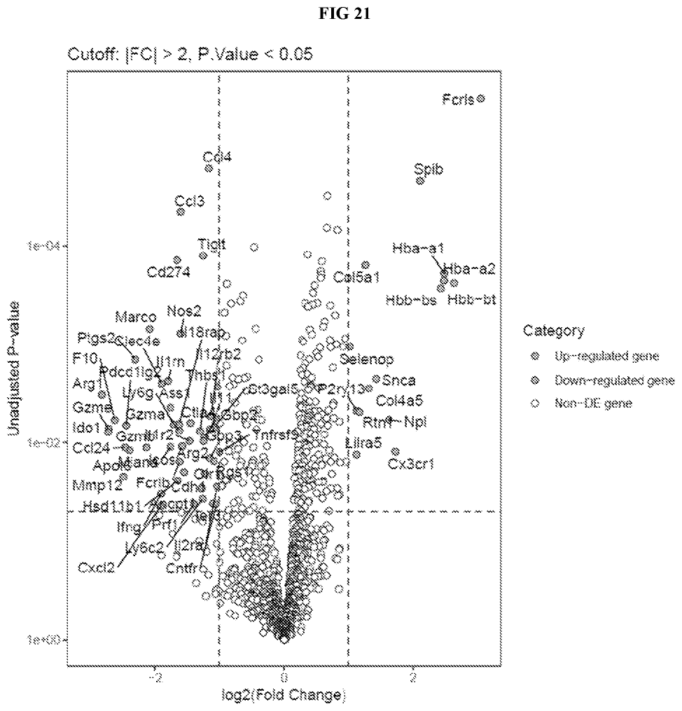

In some embodiments, the hinge domain of a human IgG antibody (e.g., IgG1, IgG2, IgG3 and IgG4) is used. In some cases, the hinge domain can contain amino acid substitutions as well. For example, as shown in SEQ ID NO:25 of FIG. 21, a hinge domain from IgG4 comprising a S228P variant is used.

In some embodiments, the domain linker is a combination of a hinge domain and a flexible linker, such as an IgG4 hinge with a S228P with a GGSGGGGS linker as well.

In one embodiment, the linker is from about 1 to 50 amino acids in length, preferably about 5 to 20 amino acids in length.

In some embodiments, a domain linker is used to link the sPD-1 variant domain with the (variant) Fc domain. In some embodiments, said domain linker comprises the amino acid sequence selected from the group consisting of SEQ ID NO:12, SEQ ID NO:13, SEQ ID NO: 14, SEQ ID NO:15, SEQ ID NO:16, SEQ ID NO:17, SEQ ID NO:18, SEQ ID NO:19, SEQ ID NO:20, and SEQ ID NO:21. In some embodiments, said domain linker comprises the amino acid sequence of SEQ ID NO:12. In some embodiments, said domain linker comprises the amino acid sequence of SEQ ID NO:13. In some embodiments, said domain linker comprises the amino acid sequence of SEQ ID NO:14. In some embodiments, said domain linker comprises the amino acid sequence of SEQ ID NO:15. In some embodiments, said domain linker comprises the amino acid sequence of SEQ ID NO:16. In some embodiments, said domain linker comprises the amino acid sequence of SEQ ID NO:17. In some embodiments, said domain linker comprises the amino acid sequence of SEQ ID NO:18. In some embodiments, said domain linker comprises the amino acid sequence of SEQ ID NO:19. In some embodiments, said domain linker comprises the amino acid sequence of SEQ ID NO:20. In some embodiments, said domain linker comprises the amino acid sequence of SEQ ID NO:21.

In some embodiments, a domain linker is used to link the IL-15 domain with the (variant) Fc domain. In some embodiments, said domain linker comprises the amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21. In some embodiments, said domain linker comprises the amino acid sequence of SEQ ID NO:12. In some embodiments, said domain linker comprises the amino acid sequence of SEQ ID NO:13. In some embodiments, said domain linker comprises the amino acid sequence of SEQ ID NO:14. In some embodiments, said domain linker comprises the amino acid sequence of SEQ ID NO:15. In some embodiments, said domain linker comprises the amino acid sequence of SEQ ID NO:16. In some embodiments, said domain linker comprises the amino acid sequence of SEQ ID NO:17. In some embodiments, said domain linker comprises the amino acid sequence of SEQ ID NO:18. In some embodiments, said domain linker comprises the amino acid sequence of SEQ ID NO:19. In some embodiments, said domain linker comprises the amino acid sequence of SEQ ID NO:20. In some embodiments, said domain linker comprises the amino acid sequence of SEQ ID NO:21.

In some embodiments, a domain linker is used to link the IL-15Rα sushi domain with the (variant) Fc domain. In some embodiments, said domain linker comprises the amino acid sequence selected from the group consisting of SEQ ID NO:12, SEQ ID NO:13, SEQ ID NO: 14, SEQ ID NO:15, SEQ ID NO:16, SEQ ID NO:17, SEQ ID NO:18, SEQ ID NO:19, SEQ ID NO:20, and SEQ ID NO:21. In some embodiments, said domain linker comprises the amino acid sequence of SEQ ID NO:12. In some embodiments, said domain linker comprises the amino acid sequence of SEQ ID NO:13. In some embodiments, said domain linker comprises the amino acid sequence of SEQ ID NO:14. In some embodiments, said domain linker comprises the amino acid sequence of SEQ ID NO:15. In some embodiments, said domain linker comprises the amino acid sequence of SEQ ID NO:16. In some embodiments, said domain linker comprises the amino acid sequence of SEQ ID NO:17. In some embodiments, said domain linker comprises the amino acid sequence of SEQ ID NO:18. In some embodiments, said domain linker comprises the amino acid sequence of SEQ ID NO:19. In some embodiments, said domain linker comprises the amino acid sequence of SEQ ID NO:20. In some embodiments, said domain linker comprises the amino acid sequence of SEQ ID NO:21.

In some embodiments, a domain linker is used to link the IL-15 domain with the IL-15Ra sushi domain. In some embodiments, said domain linker comprises the amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21. In some embodiments, said domain linker comprises the amino acid sequence of SEQ ID NO:12. In some embodiments, said domain linker comprises the amino acid sequence of SEQ ID NO:13. In some embodiments, said domain linker comprises the amino acid sequence of SEQ ID NO:14. In some embodiments, said domain linker comprises the amino acid sequence of SEQ ID NO:15. In some embodiments, said domain linker comprises the amino acid sequence of SEQ ID NO:16. In some embodiments, said domain linker comprises the amino acid sequence of SEQ ID NO:17. In some embodiments, said domain linker comprises the amino acid sequence of SEQ ID NO:18. In some embodiments, said domain linker comprises the amino acid sequence of SEQ ID NO:19. In some embodiments, said domain linker comprises the amino acid sequence of SEQ ID NO:20. In some embodiments, said domain linker comprises the amino acid sequence of SEQ ID NO:21.

In some embodiments, a domain linker is used to link the sPD-1 variant domain with the IL-15 domain, wherein said domain linker comprises the amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21. In some embodiments, said domain linker comprises the amino acid sequence of SEQ ID NO:12. In some embodiments, said domain linker comprises the amino acid sequence of SEQ ID NO:13. In some embodiments, said domain linker comprises the amino acid sequence of SEQ ID NO:14. In some embodiments, said domain linker comprises the amino acid sequence of SEQ ID NO:15. In some embodiments, said domain linker comprises the amino acid sequence of SEQ ID NO:16. In some embodiments, said domain linker comprises the amino acid sequence of SEQ ID NO:17. In some embodiments, said domain linker comprises the amino acid sequence of SEQ ID NO:18. In some embodiments, said domain linker comprises the amino acid sequence of SEQ ID NO:19. In some embodiments, said domain linker comprises the amino acid sequence of SEQ ID NO:20. In some embodiments, said domain linker comprises the amino acid sequence of SEQ ID NO:21.

In some embodiments, a domain linker is used to link the sPD-1 variant domain with the IL-15Rα domain, wherein said domain linker comprises the amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21. In some embodiments, said domain linker comprises the amino acid sequence of SEQ ID NO:12. In some embodiments, said domain linker comprises the amino acid sequence of SEQ ID NO:13. In some embodiments, said domain linker comprises the amino acid sequence of SEQ ID NO:14. In some embodiments, said domain linker comprises the amino acid sequence of SEQ ID NO:15. In some embodiments, said domain linker comprises the amino acid sequence of SEQ ID NO:16. In some embodiments, said domain linker comprises the amino acid sequence of SEQ ID NO:17. In some embodiments, said domain linker comprises the amino acid sequence of SEQ ID NO:18. In some embodiments, said domain linker comprises the amino acid sequence of SEQ ID NO:19. In some embodiments, said domain linker comprises the amino acid sequence of SEQ ID NO:20. In some embodiments, said domain linker comprises the amino acid sequence of SEQ ID NO:21.

In some embodiments, a domain linker is used to link the sPD-1 variant domain with the (variant) Fc domain, wherein said domain linker comprises the amino acid sequence selected from the group consisting of SEQ ID NO:19, SEQ ID NO:20, and SEQ ID NO:21. In some embodiments, a domain linker is used to link the IL-15 domain with the (variant) Fc domain, wherein said domain linker comprises the amino acid sequence selected from the group consisting of SEQ ID NO:19, SEQ ID NO:20, and SEQ ID NO:21. In some embodiments, a domain linker is used to link the IL-15Rα sushi domain with the (variant) Fc domain, wherein said domain linker comprises the amino acid sequence selected from the group consisting of SEQ ID NO:19, SEQ ID NO:20, and SEQ ID NO:21.

In some embodiments, a domain linker is used to link the IL-15 domain with the IL-15Rα sushi domain, wherein said domain linker comprises the amino acid sequence selected from the group consisting of NO:12, SEQ ID NO:13, SEQ ID NO:14, SEQ ID NO:15, SEQ ID NO:16, SEQ ID NO:17, and SEQ ID NO:18. In some embodiments, a domain linker is used to link the IL-15 domain with the IL-15Rα sushi domain, wherein said domain linker comprises the amino acid sequence of SEQ ID NO:15 or SEQ ID NO:18.

In some embodiments, a first domain linker is used to link the sPD-1 variant domain with the (variant) Fc domain, wherein said first domain linker comprises the amino acid sequence selected from the group consisting of SEQ ID NO:19, SEQ ID NO:20, and SEQ ID NO:21; a second domain linker is used to link the IL-15 domain or the IL-15Rα sushi domain with the (variant) Fc domain, wherein said second domain linker comprises the amino acid sequence selected from the group consisting of SEQ ID NO:19, SEQ ID NO:20, and SEQ ID NO:21; and a third domain linker is used to link the IL-15 domain with the IL-15Rα sushi domain, wherein said third domain linker comprises the amino acid sequence selected from the group consisting of NO:12, SEQ ID NO:13, SEQ ID NO:14, SEQ ID NO:15, SEQ ID NO:16, SEQ ID NO:17, and SEQ ID NO:18.

In some embodiments, a first domain linker is used to link the sPD-1 variant domain with the (variant) Fc domain, wherein said first domain linker comprises the amino acid sequence selected from the group consisting of SEQ ID NO:19, SEQ ID NO:20, and SEQ ID NO:21; a second domain linker is used to link the IL-15 domain or the IL-15Rα sushi domain with the (variant) Fc domain, wherein said second domain linker comprises the amino acid sequence selected from the group consisting of SEQ ID NO:19, SEQ ID NO:20, and SEQ ID NO:21; and a third domain linker is used to link the IL-15 domain with the IL-15Rα sushi domain, wherein said third domain linker comprises the amino acid sequence of SEQ ID NO: 15 or SEQ ID NO:18.

### D. Exemplary Embodiments of the Disclosure

The bispecific Fc fusion proteins of the present disclosure comprise four domains: a) an IL-15Rα sushi domain; b) an IL-15 domain; c) an Fc domain; and d) a soluble PD-1 (sPD-1) variant domain; and optionally further comprises domain linkers.

In some embodiments, the bispecific Fc fusion proteins comprise one domain linker. In some embodiments, the bispecific Fc fusion proteins comprise two domain linkers. In some embodiments, the bispecific Fc fusion proteins comprise a first domain linker, a second domain linker, and a third domain linker.

In some embodiments, the first domain linker as described herein is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, (GS)n, (GSGGS)n, (GGGGS)n, and (GGGS)n, wherein n is selected from the group consisting of 1, 2, 3, 4 and 5.

In some embodiments, the second domain linker as described herein is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, (GS)n, (GSGGS)n, (GGGGS)n, and (GGGS)n, wherein n is selected from the group consisting of 1, 2, 3, 4 and 5.

In some embodiments, the third domain linker as described herein is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, (GS)n, (GSGGS)n, (GGGGS)n, and (GGGS)n, wherein n is selected from the group consisting of 1, 2, 3, 4 and 5.

In some embodiments, a domain linker is used to link the sPD-1 variant domain with the (variant) Fc domain, wherein said domain linker comprises the amino acid sequence selected from the group consisting of SEQ ID NO:19, SEQ ID NO:20, and SEQ ID NO:21. In some embodiments, the domain linker used to link the sPD-1 variant domain with the (variant) Fc domain comprises the amino acid sequence of SEQ ID NO:19. In some embodiments, the domain linker used to link the sPD-1 variant domain with the (variant) Fc domain comprises the amino acid sequence of SEQ ID NO:20. In some embodiments, the domain linker used to link the sPD-1 variant domain with the (variant) Fc domain comprises the amino acid sequence of SEQ ID NO:21.

In some embodiments, a domain linker is used to link the IL-15 domain with the (variant) Fc domain, wherein said domain linker comprises the amino acid sequence selected from the group consisting of SEQ ID NO:19, SEQ ID NO:20, and SEQ ID NO:21. In some embodiments, the domain linker used to link the IL-15 variant domain with the (variant) Fc domain comprises the amino acid sequence of SEQ ID NO:19. In some embodiments, the domain linker used to link the IL-15 variant domain with the (variant) Fc domain comprises the amino acid sequence of SEQ ID NO:20. In some embodiments, the domain linker used to link the IL-15 variant domain with the (variant) Fc domain comprises the amino acid sequence of SEQ ID NO:21.

In some embodiments, a domain linker is used to link the IL-15Rα sushi domain with the (variant) Fc domain, wherein said domain linker comprises the amino acid sequence selected from the group consisting of SEQ ID NO:19, SEQ ID NO:20, and SEQ ID NO:21. In some embodiments, the domain linker used to link the IL-15Rα sushi domain with the (variant) Fc domain comprises the amino acid sequence of SEQ ID NO:19. In some embodiments, the domain linker used to link the IL-15Ra sushi domain with the (variant) Fc domain comprises the amino acid sequence of SEQ ID NO:20. In some embodiments, the domain linker used to link the IL-15Rα sushi domain with the (variant) Fc domain comprises the amino acid sequence of SEQ ID NO:21.

In some embodiments, a domain linker is used to link the IL-15 domain with the IL-15Ra sushi domain, wherein said domain linker comprises the amino acid sequence selected from the group consisting of NO:12, SEQ ID NO:13, SEQ ID NO:14, SEQ ID NO:15, SEQ ID NO:16, SEQ ID NO:17, and SEQ ID NO:18. In some embodiments, the domain linker used to link the IL-15 domain with the IL-15Rα sushi domain comprises the amino acid sequence of SEQ ID NO:12. In some embodiments, the domain linker used to link the IL-15 domain with the IL-15Rα sushi domain comprises the amino acid sequence of SEQ ID NO:13. In some embodiments, the domain linker used to link the IL-15 domain with the IL-15Rα sushi domain comprises the amino acid sequence of SEQ ID NO:14. In some embodiments, the domain linker used to link the IL-15 domain with the IL-15Rα sushi domain comprises the amino acid sequence of SEQ ID NO:15. In some embodiments, the domain linker used to link the IL-15 domain with the IL-15Rα sushi domain comprises the amino acid sequence of SEQ ID NO:16. In some embodiments, the domain linker used to link the IL-15 domain with the IL-15Rα sushi domain comprises the amino acid sequence of SEQ ID NO:17. In some embodiments, the domain linker used to link the IL-15 domain with the IL-15Rα sushi domain comprises the amino acid sequence of SEQ ID NO:18.

In some embodiments, a domain linker is used to link the sPD-1 variant domain with the IL-15 domain, wherein said domain linker comprises the amino acid sequence selected from the group consisting of SEQ ID NO:19, SEQ ID NO:20, and SEQ ID NO:21. In some embodiments, the domain linker used to link the sPD-1 variant domain with the IL-15 domain comprises the amino acid sequence of SEQ ID NO:19. In some embodiments, the domain linker used to link the sPD-1 variant domain with the IL-15 domain comprises the amino acid sequence of SEQ ID NO:20. In some embodiments, the domain linker used to link the sPD-1 variant domain with the IL-15 domain comprises the amino acid sequence of SEQ ID NO:21.

In some embodiments, a domain linker is used to link the sPD-1 variant domain with the IL-15Rα domain, wherein said domain linker comprises the amino acid sequence selected from the group consisting of SEQ ID NO:19, SEQ ID NO:20, and SEQ ID NO:21. In some embodiments, the domain linker used to link the sPD-1 variant domain with the IL-15Rα domain comprises the amino acid sequence of SEQ ID NO:19. In some embodiments, the domain linker used to link the sPD-1 variant domain with the IL-15Rα domain comprises the amino acid sequence of SEQ ID NO:20. In some embodiments, the domain linker used to link the sPD-1 variant domain with the IL-15Ra domain comprises the amino acid sequence of SEQ ID NO:21.

In some embodiments, one domain linker is used to link the sPD-1 variant domain with the (variant) Fc domain, wherein said domain linker comprises the amino acid sequence selected from the group consisting of SEQ ID NO:19, SEQ ID NO:20, and SEQ ID NO:21; an additional domain linker is used to link the IL-15 domain or the IL-15Rα sushi domain with the (variant) Fc domain, wherein said additional domain linker comprises the amino acid sequence selected from the group consisting of SEQ ID NO: 19, SEQ ID NO:20, and SEQ ID NO:21; and a further domain linker is used to link the IL-15 domain with the IL-15Rα sushi domain, wherein said further domain linker comprises the amino acid sequence selected from the group consisting of NO:12, SEQ ID NO:13, SEQ ID NO:14, SEQ ID NO:15, SEQ ID NO:16, SEQ ID NO:17, and SEQ ID NO:18.

In some embodiments, one domain linker is used to link the sPD-1 variant domain with the (variant) Fc domain, wherein said domain linker comprises the amino acid sequence selected from the group consisting of SEQ ID NO:19, SEQ ID NO:20, and SEQ ID NO:21; an additional domain linker is used to link the IL-15 domain or the IL-15Rα sushi domain with the (variant) Fc domain, wherein said additional domain linker comprises the amino acid sequence selected from the group consisting of SEQ ID NO: 19, SEQ ID NO:20, and SEQ ID NO:21; and a further domain linker is used to link the IL-15 domain with the IL-15Rα sushi domain, wherein said further domain linker comprises the amino acid sequence of SEQ ID NO:15.

In some embodiments, one domain linker is used to link the sPD-1 variant domain with the (variant) Fc domain, wherein said domain linker comprises the amino acid sequence selected from the group consisting of SEQ ID NO:19, SEQ ID NO:20, and SEQ ID NO:21; an additional domain linker is used to link the IL-15 domain or the IL-15Rα sushi domain with the (variant) Fc domain, wherein said additional domain linker comprises the amino acid sequence selected from the group consisting of SEQ ID NO: 19, SEQ ID NO:20, and SEQ ID NO:21; and a further domain linker is used to link the IL-15 domain with the IL-15Rα sushi domain, wherein said further domain linker comprises the amino acid sequence of SEQ ID NO:18.

In one embodiment, the bispecific Fc fusion protein comprises a) an IL-15Rα sushi domain; b) an IL-15 domain; c) an Fc domain; d) a soluble PD-1 (sPD-1) variant domain; a first domain linker, a second domain linker, and a third domain linker.

In one embodiment, the bispecific Fc fusion protein comprises, from N- to C-terminus: a) the IL-15Rα sushi domain; b) the first domain linker; c) the IL-15 domain; d) the second domain linker; e) the Fc domain; f) the third domain linker; and g) the sPD-1 variant domain.

In a further embodiment, the bispecific Fc fusion protein comprises, from N- to C-terminus: a) the IL-15 domain; b) the first domain linker; c) the IL-15Rα sushi domain; d) the second domain linker; e) the Fc domain; f) the third domain linker; and g) the sPD-1 variant domain.

In an additional embodiment, the bispecific Fc fusion protein comprises, from N- to C-terminus: a) the sPD-1 variant domain; b) the first domain linker; c) the Fc domain; d) the second domain linker; e) the IL-15 domain; f) the third domain linker; and g) the IL-15Rα sushi domain.

In a further embodiment, the bispecific Fc fusion protein comprises, from N- to C-terminus: a) the sPD-1 variant domain; b) the first domain linker; c) the Fc domain; d) the second domain linker; e) the IL-15Rα sushi domain; f) the third domain linker; and g) the IL-15 domain.

In an additional embodiment, the bispecific Fc fusion protein comprises, from N- to C-terminus: a) the IL-15Rα sushi domain; b) the first domain linker; c) the IL-15 domain; d) the second domain linker; e) the sPD-1 variant domain; f) the third domain linker; and g) the Fc domain.

In a further embodiment, the bispecific Fc fusion protein comprises, from N- to C-terminus: a) the IL-15 domain; b) the first domain linker; c) the IL-15Rα sushi domain; d) the second domain linker; e) the sPD-1 variant domain; f) the third domain linker; and g) the Fc domain.

In an additional embodiment, the bispecific Fc fusion protein comprises, from N- to C-terminus: a) the sPD-1 variant domain; b) the first domain linker; c) the IL-15 domain; d) the second domain linker; e) the IL-15Rα sushi domain; f) the third domain linker; and g) the Fc domain.

In a further embodiment, the bispecific Fc fusion protein comprises, from N- to C-terminus: a) the sPD-1 variant domain; b) the first domain linker; c) the IL-15Rα sushi domain; d) the second domain linker; e) the IL-15 domain; f) the third domain linker; and g) the Fc domain.

In an additional embodiment, the bispecific Fc fusion protein comprises, from N- to C-terminus: a) the Fc domain; b) the first domain linker; c) the IL-15 domain; d) the second domain linker; e) the IL-15Rα sushi domain; f) the third domain linker; and g) the sPD-1 variant domain.

In a further embodiment, the bispecific Fc fusion protein comprises, from N- to C-terminus: a) the Fc domain; b) the first domain linker; c) the IL-15Rα sushi domain; d) the second domain linker; e) the IL-15 domain; f) the third domain linker; and g) the sPD-1 variant domain.

In an additional embodiment, the bispecific Fc fusion protein comprises, from N- to C-terminus: a) the Fc domain; b) the first domain linker; c) the sPD-1 variant domain; d) the second domain linker; e) the IL-15 domain; f) the third domain linker; and g) the IL-15Rα sushi domain.

In a further embodiment, the bispecific Fc fusion protein comprises, from N- to C-terminus: a) the Fc domain; b) the first domain linker; c) the sPD-1 variant domain; d) the second domain linker; e) the IL-15Rα sushi domain; f) the third domain linker; and g) the IL-15 domain.

In some embodiments, the bispecific Fc fusion protein comprises the a), b), c), d), e), f) and g) domains as disclosed herein, wherein the sPD-1 variant domain comprises one or more amino acid substitutions at positions corresponding to positions selected from the group consisting of positions 38, 63, 65, 92, 100, 103, 108 and 116 of SEQ ID NO: 1. In some embodiments, the sPD-1 variant domain comprises an amino acid substitution at a position corresponding to position 38 of SEQ ID NO: 1. In some embodiments, the sPD-1 variant domain comprises an amino acid substitution at a position corresponding to position 63 of SEQ ID NO: 1. In some embodiments, the sPD-1 variant domain comprises an amino acid substitution at a position corresponding to position 65 of SEQ ID NO: 1. In some embodiments, the sPD-1 variant domain comprises an amino acid substitution at a position corresponding to position 92 of SEQ ID NO: 1. In some embodiments, the sPD-1 variant domain comprises an amino acid substitution at a position corresponding to position 100 of SEQ ID NO: 1. In some embodiments, the sPD-1 variant domain comprises an amino acid substitution at a position corresponding to position 103 of SEQ ID NO: 1. In some embodiments, the sPD-1 variant domain comprises an amino acid substitution at a position corresponding to position 108 of SEQ ID NO: 1. In some embodiments, the sPD-1 variant domain comprises an amino acid substitution at a position corresponding to position 116 of SEQ ID NO: 1. In some embodiments, the sPD-1 variant domain comprises amino acid substitutions occurring at two of said positions. In some embodiments, the sPD-1 variant domain comprises amino acid substitutions occurring at three of said positions. In some embodiments, the sPD-1 variant domain comprises amino acid substitutions occurring at four of said positions. In some embodiments, the sPD-1 variant domain comprises amino acid substitutions occurring at five of said positions. In some embodiments, the sPD-1 variant domain comprises amino acid substitutions occurring at six of said positions. In some embodiments, the sPD-1 variant domain comprises amino acid substitutions occurring at seven of said positions. In some embodiments, the sPD-1 variant domain comprises amino acid substitutions occurring at eight of said positions. In some embodiments, the sPD-1 variant domain comprises an amino acid sequence having at least 96% sequence identity to SEQ ID NO:1. In some embodiments, the sPD-1 variant domain comprises an amino acid sequence having at least 97% sequence identity to SEQ ID NO:1. In some embodiments, the sPD-1 variant domain comprises an amino acid sequence having at least 98% sequence identity to SEQ ID NO:1. In some embodiments, the sPD-1 variant domain comprises an amino acid sequence having at least 99% sequence identity to SEQ ID NO:1.

In some embodiments, the bispecific Fc fusion protein comprises the a), b), c), d), e), f) and g) domains as disclosed herein, wherein the sPD-1 variant domain comprises one or more amino acid substitutions selected from the group consisting of S38G, S63G, P65L, N92S, G100S, S103V, A108I, and A116V of SEQ ID NO:1. In some embodiments, the sPD-1 variant domain comprises a set of amino acid substitutions N92S/G100S/S103V/A108I/A116V of SEQ ID NO:1. In some embodiments, the sPD-1 variant domain comprises a set of amino acid substitutions S38G/S63G/P65L/N92S/G100S/S103V/A1081/A116V. In some embodiments, the sPD-1 variant domain comprises a set of amino acid substitutions S38G/S63G/P65L/G100S/S103V/A108I/A116V. In some embodiments, the sPD-1 variant domain comprises a set of amino acid substitutions P65L/G100S/S103V/A108I/A116V. In some embodiments, the sPD-1 variant domain comprises a set of amino acid substitutions S63G/G100S/S103V/A108I/A116V. In some embodiments, the sPD-1 variant domain comprises a set of amino acid substitutions S63G/P65L/G100S/S103V/A1081/A116V. In some embodiments, the sPD-1 variant domain comprises a set of amino acid substitutions G100S/S103V/A108I/A116V. In some embodiments, the sPD-1 variant domain comprises a set of amino acid substitutions G100S/S103V/A108I.

In some embodiments, the bispecific Fc fusion protein comprises the a), b), c), d), e), f) and g) domains as disclosed herein, wherein the sPD-1 variant domain comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8 and SEQ ID NO:9. In some embodiments, the sPD-1 variant domain comprises the amino acid sequence of SEQ ID NO:2. In some embodiments, the sPD-1 variant domain comprises the amino acid sequence of SEQ ID NO:3. In some embodiments, the sPD-1 variant domain comprises the amino acid sequence of SEQ ID NO:4. In some embodiments, the sPD-1 variant domain comprises the amino acid sequence of SEQ ID NO:5. In some embodiments, the sPD-1 variant domain comprises the amino acid sequence of SEQ ID NO:6. In some embodiments, the sPD-1 variant domain comprises the amino acid sequence of SEQ ID NO:7. In some embodiments, the sPD-1 variant domain comprises the amino acid sequence of SEQ ID NO:8. In some embodiments, the sPD-1 variant domain comprises the amino acid sequence of SEQ ID NO:9.

In some embodiments, the bispecific Fc fusion protein comprises the a), b), c), d), e), f) and g) domains as disclosed herein, wherein the IL-15 domain comprises the amino acid sequence of SEQ ID NO:10.

In some embodiments, the bispecific Fc fusion protein comprises the a), b), c), d), e), f) and g) domains as disclosed herein, wherein the IL-15Rα sushi domain comprises the amino acid sequence of SEQ ID NO:11.

In some embodiments, the bispecific Fc fusion protein comprises the a), b), c), d), e), f) and g) domains as disclosed herein, wherein the IL-15 domain comprises the amino acid sequence of SEQ ID NO:10 and the IL-15Rα sushi domain comprises the amino acid sequence of SEQ ID NO:11.

In some embodiments, the bispecific Fc fusion protein comprises the a), b), c), d), e), f) and g) domains as disclosed herein, wherein the Fc domain is a human IgG Fc domain or a variant human IgG Fc domain. In some embodiments, the human IgG Fc domain comprises hinge-CH2-CH3 of human IgG4. In some embodiments, the Fc domain is a variant human IgG Fc domain. In some embodiments, the Fc domain is a variant human IgG Fc domain comprising hinge-CH2-CH3 of human IgG4 with a substitution corresponding to S228P as set forth in SEQ ID NO: 25.

In some embodiments, the bispecific Fc fusion protein comprises the a), b), c), d), e), f) and g) domains as disclosed herein, wherein the IL-15 domain is not glycosylated.

In some embodiments, the bispecific Fc fusion protein comprises the a), b), c), d), e), f) and g) domains as disclosed herein, wherein the IL-15Rα domain is not glycosylated.

In some embodiments, the bispecific Fc fusion protein comprises the a), b), c), d), e), f) and g) domains as disclosed herein, wherein the Fc domain is not glycosylated.

In some embodiments, the bispecific Fc fusion protein of the present disclosure comprise no more than one IL-15 domain.

In some embodiments, the bispecific Fc fusion protein of the present disclosure comprise no more than one IL-15Rα domain.

In some embodiments, the bispecific Fc fusion protein of the present disclosure comprise no more than one sPD-1 variant domain.

In some embodiments, the bispecific Fc fusion protein of the present disclosure comprise no more than one Fc domain.

In some embodiments, the bispecific Fc fusion protein of the present disclosure comprise no more than one IL-15 domain, no more than one IL-15Rα domain, no more than one sPD-1 variant domain, and no more than one Fc domain.

In some embodiments, the bispecific Fc fusion protein as disclosed herein comprising the amino acid sequence of SEQ ID NO:26.

In some embodiments, the bispecific Fc fusion protein as disclosed herein comprising the amino acid sequence of SEQ ID NO:27.

In some embodiments, the bispecific Fc fusion protein as disclosed herein comprising the amino acid sequence of SEQ ID NO:28.

In some embodiments, the bispecific Fc fusion protein as disclosed herein comprising the amino acid sequence of SEQ ID NO:29.

In some embodiments, the bispecific Fc fusion protein as disclosed herein comprising the amino acid sequence of SEQ ID NO:30.

In some embodiments, the bispecific Fc fusion protein as disclosed herein comprising the amino acid sequence of SEQ ID NO:31.

In some embodiments, the bispecific Fc fusion protein as disclosed herein comprising the amino acid sequence of SEQ ID NO:32.

In some embodiments, the bispecific Fc fusion protein as disclosed herein comprising the amino acid sequence of SEQ ID NO:33.

In some embodiments, the bispecific Fc fusion protein as disclosed herein comprising the amino acid sequence of SEQ ID NO:34.

In some embodiments, the bispecific Fc fusion protein as disclosed herein comprising the amino acid sequence of SEQ ID NO:35.

In some embodiments, the bispecific Fc fusion protein as disclosed herein comprising the amino acid sequence of SEQ ID NO:36.

In some embodiments, the bispecific Fc fusion protein as disclosed herein comprising the amino acid sequence of SEQ ID NO:37.

In some embodiments, the bispecific Fc fusion protein as disclosed herein comprising the amino acid sequence of SEQ ID NO:62.

In some embodiments, the bispecific Fc fusion protein as disclosed herein comprising the amino acid sequence of SEQ ID NO:63.

In some embodiments, the bispecific Fc fusion protein as disclosed herein comprising the amino acid sequence of SEQ ID NO:64.

In some embodiments, the bispecific Fc fusion protein as disclosed herein comprising the amino acid sequence of SEQ ID NO:65.

In some embodiments, the bispecific Fc fusion protein as disclosed herein comprising the amino acid sequence of SEQ ID NO:66.

In some embodiments, the bispecific Fc fusion protein as disclosed herein comprising the amino acid sequence of SEQ ID NO:67.

In some embodiments, the bispecific Fc fusion protein as disclosed herein comprising the amino acid sequence of SEQ ID NO:68.

In some embodiments, the bispecific Fc fusion protein as disclosed herein comprising the amino acid sequence of SEQ ID NO:69.

In some embodiments, the bispecific Fc fusion protein as disclosed herein comprising the amino acid sequence of SEQ ID NO:70.

In some embodiments, the bispecific Fc fusion protein as disclosed herein comprising the amino acid sequence of SEQ ID NO:71.

In some embodiments, the bispecific Fc fusion protein as disclosed herein comprising the amino acid sequence of SEQ ID NO:72.

In some embodiments, the present disclosure provides a pharmaceutical composition comprising the bispecific Fc fusion protein as disclosed herein and a pharmaceutically acceptable carrier, excipient and/or stabilizer.

In some embodiments, the bispecific Fc fusion protein of the present disclosure does not comprise IL-12, a mutant thereof or a fragment thereof.

In some embodiments, the bispecific Fc fusion protein of the present disclosure does not comprise ECD of IL-12Rβ, a mutant thereof or a fragment thereof.

In some embodiments, the bispecific Fc fusion protein of the present disclosure does not comprise ECD of IL-12Ry, a mutant thereof or a fragment thereof.

In some embodiments, the bispecific Fc fusion protein of the present disclosure does not comprise TGFβ binding peptide, a mutant thereof or a fragment thereof.

In some embodiments, the bispecific Fc fusion protein of the present disclosure does not comprise TGFβR2, a mutant thereof or a fragment thereof.

In some embodiments, the bispecific Fc fusion protein of the present disclosure does not comprise TGFβR3, a mutant thereof or a fragment thereof.

In some embodiments, the bispecific Fc fusion protein of the present disclosure does not comprise soluble TGF-β regulatory peptides, a mutant thereof or a fragment thereof, or precursors capable of forming soluble TGF-beta regulatory peptides.

In some embodiments, the bispecific Fc fusion protein of the present disclosure does not comprise soluble Gal9 binding peptide. In some embodiments, the bispecific Fc fusion protein of the present disclosure does not comprise extracellular domain of Tim3, a mutant thereof or a fragment thereof.

In some embodiments, the bispecific Fc fusion protein of the present disclosure does not comprise CD80, a mutant thereof or a fragment thereof.

In some embodiments, the bispecific Fc fusion protein of the present disclosure does not comprise a transmembrane region of CD8 or CD28.

In some embodiments, the bispecific Fc fusion protein of the present disclosure does not comprise an intracellular signaling domain which is a polypeptide obtained by fusing a CD3ζ signaling transduction region with a 4-1BB (CD137) signaling transduction region, or a polypeptide obtained by fusion of a CD3ζ signal transduction region and a CD28 signal transduction region.

In some embodiments, the bispecific Fc fusion protein of the present disclosure does not comprise IL4, a mutant thereof or a fragment thereof.

In some embodiments, the bispecific Fc fusion protein of the present disclosure does not comprise IL4Rα, a mutant thereof or a fragment thereof.

In some embodiments, the bispecific Fc fusion protein of the present disclosure does not comprise IL-12, a mutant thereof or a fragment thereof.

In some embodiments, the bispecific Fc fusion protein of the present disclosure does not comprise a transmembrane domain from an alpha chain of IL-7 receptor or a variant thereof or a fragment thereof.

In some embodiments, the bispecific Fc fusion protein of the present disclosure does not comprise IL2, a mutant thereof or a fragment thereof.

In some embodiments, the bispecific Fc fusion protein of the present disclosure does not comprise IL2Rα, a mutant thereof or a fragment thereof.

In some embodiments, the bispecific Fc fusion protein of the present disclosure does not comprise IL10, a mutant thereof or a fragment thereof.

In some embodiments, the bispecific Fc fusion protein of the present disclosure does not comprise CD16, a mutant thereof or a fragment thereof.

In some embodiments, the bispecific Fc fusion protein of the present disclosure does not comprise CD32, a mutant thereof or a fragment thereof.

In some embodiments, the bispecific Fc fusion protein of the present disclosure does not comprise CD64, a mutant thereof or a fragment thereof.

In some embodiments, the bispecific Fc fusion protein of the present disclosure does not comprise any one or a combination of antiCD19-ScFv, AntiMHC/GP100-VHH, AntiMHC/WTI-VH, AntiCD20-ScFv, and AntiCD22-ScFv.

In some embodiments, the bispecific Fc fusion protein of the present disclosure does not comprise an antigen binding domain that binds glioma-associated antigens, carcinoembryonic antigen (CEA), P-human chorionic gonadotropin, a-Fetoprotein (AFP), lectin-reactive AFP, thyroglobulin, RAGE-1, MN-CA IX, human telomerase reverse transcriptase, RU1, RU2 (AS), intestinal carboxylesterase, mut hsp70-2, M-CSF, prostatase, prostate-specific antigen (PSA), PAP, NY-ESO-1, LAGE-la, p53, prostein, PSMA, Her2/neu, survivin and telomerase, Prostate-Cancer Tumor Antigen-1 (PCTA-1), MAGE, ELF2M, Neutrophil Elastase, Ephrin B2, CD22, Insulin Growth Factor (IGF)-I, IGF-II, IGF-I Receptor or Mesothelium Vegetarian.

In some embodiments, the bispecific Fc fusion protein of the present disclosure does not comprise a transmembrane domain, or portion thereof, from an endogenous polypeptide, where the endogenous polypeptide is selected from the group of: an a chain of a T cell receptor, a β chain of the T cell receptor, a ζ chain of the T cell receptor, CD28 (also known as Tp44), CD3ε, CD3δ, CD3γ, CD33, CD37 (also known as GP52-40 or TSPAN26), CD64 (also known as FCGR1A), CD80 (also known as B7, B7-1, B7.1, BB1, CD28LG, CD28LG1, and LAB7), CD45 (also known as PTPRC, B220, CD45R, GP180, L-CA, LCA, LY5, T200, and protein tyrosine phosphatase, receptor type C), CD4, CD5 (also known as LEU1 and T1), CD8a (also known as Leu2, MAL, and p32), CD9 (also known as BTCC-1, DRAP-27, MIC3, MRP-1, TSPAN-29, and TSPAN29), CD16 (also known as FCGR3 andFCG3), CD22 (also known as SIGLEC-2 and SIGLEC2), CD86 (also known as B7-2, B7.2, B70, CD28LG2, and LAB72), CD134 (also known as TNFRSF4, ACT35, RP5-902P8.3, IMD16, OX40, TXGPIL, and tumor necrosis factor receptor superfamily member 4), CD137 (also known as TNFRSF9, 4-1BB, CDwi37, ILA, and tumor necrosis factor receptor superfamily member 9), CD27 (also known as 5152, 5152.LPFS2, T14, TNFRSF7, and Tp55), and CD152 (also known as CTLA4, ALPS5, CELIAC3, CTLA-4, GRD4, GSE, IDDM12, and cytotoxic T-lymphocyte associated protein 4).

In some embodiments, the bispecific Fc fusion protein of the present disclosure does not comprise any chimeric antigen receptor (CAR) polypeptide.

In some embodiments, the bispecific Fc fusion protein of the present disclosure does not comprise a domain comprising a single-chain variable fragment (scFv).

In some embodiments, the bispecific Fc fusion protein of the present disclosure does not comprise a measles virus hemagglutinin (MVH) polypeptide, a measles virus fusion (MVF) polypeptide, or a vesicular stomatitis virus glycoprotein (VSVG) polypeptide.

In some embodiments, the bispecific Fc fusion protein of the present disclosure does not comprise a 4-1BB ligand (4-1BBL) polypeptide, a OX40 ligand (OX40L) polypeptide, a CD40 ligand (CD40L) polypeptide, or a granulocyte-macrophage colony-stimulating factor (GM-CSF) polypeptide.

In some embodiments, the bispecific Fc fusion protein of the present disclosure does not comprise capsid hexon polypeptides of an Ad strain Ad6 or capsid hexon hypervaribale region (HVR) polypeptide from Ad strain Ad57.

In some embodiments, the bispecific Fc fusion protein of the present disclosure does not comprise vitamin K-dependent gamma-carboxyglutamic domain of a factor X single-chain antibody polypeptide.

In some embodiments, the bispecific Fc fusion protein of the present disclosure does not comprise a carrier moiety which is a PEG molecule, an albumin, an albumin fragment, or an antibody (variant) or an antigen-binding fragment thereof.

In some embodiments, the bispecific Fc fusion protein of the present disclosure does not comprise an antibody or an antigen-binding fragment thereof that specifically binds to one or more antigens selected from PD-1, CTLA-4, LAG-3, TIM-3, CD47, and TIGIT.

### E. Nucleic Acids

The present disclosure also provides compositions comprising nucleic acids encoding the bispecific fusion proteins as disclosed herein comprising an IL-15 domain, an IL-15Rα sushi domain, an Fc domain and an sPD-1 variant domain. Such nucleic acids can encode any of the bispecific Fc fusion proteins recited in the present application.

The nucleic acids of the present disclosure may be isolated and obtained in substantial purity. Usually, the nucleic acids, either as DNA or RNA, will be obtained substantially free of other naturally occurring nucleic acid sequences, generally being at least about 50%, usually at least about 90% pure and are typically “recombinant,” e.g., flanked by one or more nucleotides with which it is not normally associated on a naturally occurring chromosome.

In some embodiments, the composition comprises a nucleic acid encoding a bispecific Fc fusion protein comprising the amino acid sequence selected from the group consisting of SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, and SEQ ID NO:72.

In some embodiments, the composition comprises a nucleic acid encoding the bispecific Fc fusion protein of SEQ ID NO:26. In some embodiments, the composition comprises a nucleic acid encoding the bispecific Fc fusion protein of SEQ ID NO:27. In some embodiments, the composition comprises a nucleic acid encoding the bispecific Fc fusion protein of SEQ ID NO:27. In some embodiments, the composition comprises a nucleic acid encoding the bispecific Fc fusion protein of SEQ ID NO:29. In some embodiments, the composition comprises a nucleic acid encoding the bispecific Fc fusion protein of SEQ ID NO: 30. In some embodiments, the composition comprises a nucleic acid encoding the bispecific Fc fusion protein of SEQ ID NO:31. In some embodiments, the composition comprises a nucleic acid encoding the bispecific Fc fusion protein of SEQ ID NO:32. In some embodiments, the composition comprises a nucleic acid encoding the bispecific Fc fusion protein of SEQ ID NO:33. In some embodiments, the composition comprises a nucleic acid encoding the bispecific Fc fusion protein of SEQ ID NO:34. In some embodiments, the composition comprises a nucleic acid encoding the bispecific Fc fusion protein of SEQ ID NO:35. In some embodiments, the composition comprises a nucleic acid encoding the bispecific Fc fusion protein of SEQ ID NO:36. In some embodiments, the composition comprises a nucleic acid encoding the bispecific Fc fusion protein of SEQ ID NO:37. In some embodiments, the composition comprises a nucleic acid encoding the bispecific Fc fusion protein of SEQ ID NO:62. In some embodiments, the composition comprises a nucleic acid encoding the bispecific Fc fusion protein of SEQ ID NO:63. In some embodiments, the composition comprises a nucleic acid encoding the bispecific Fc fusion protein of SEQ ID NO:64. In some embodiments, the composition comprises a nucleic acid encoding the bispecific Fc fusion protein of SEQ ID NO:65. In some embodiments, the composition comprises a nucleic acid encoding the bispecific Fc fusion protein of SEQ ID NO:66. In some embodiments, the composition comprises a nucleic acid encoding the bispecific Fc fusion protein of SEQ ID NO:67. In some embodiments, the composition comprises a nucleic acid encoding the bispecific Fc fusion protein of SEQ ID NO:68. In some embodiments, the composition comprises a nucleic acid encoding the bispecific Fc fusion protein of SEQ ID NO:69. In some embodiments, the composition comprises a nucleic acid encoding the bispecific Fc fusion protein of SEQ ID NO:70. In some embodiments, the composition comprises a nucleic acid encoding the bispecific Fc fusion protein of SEQ ID NO:71. In some embodiments, the composition comprises a nucleic acid encoding the bispecific Fc fusion protein of SEQ ID NO:72.

In some embodiments, the nucleic acid encodes the bispecific Fc fusion protein including a signal sequence or a signal peptide. As is known in the art, signal sequences are used to direct the expression product to the exterior of the cell. As will be appreciated by those in the art, suitable signal sequences or signal peptides for expression of the fusion proteins of the disclosure can be “matched” to the host cell used for expression. That is, when the fusion proteins of the disclosure are to be expressed in mammalian host cells such as CHO cells, for example, signal sequences from CHO cells can be used.

In some embodiments, the present disclosure provides a nucleic acid encoding a preprotein comprising a signal peptide and the Fc fusion protein as disclosed herein. In some embodiments, the signal peptide comprises an amino acid sequence having at least about 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:22 or SEQ ID NO:23. In some embodiments, the signal peptide comprises the amino acid sequence of SEQ ID NO:22. In some embodiments, the signal peptide comprises the amino acid sequence of SEQ ID NO:23.

In some embodiments, the present disclosure provides a nucleic acid encoding the preprotein comprising a signal peptide and the Fc fusion protein as disclosed herein, wherein the preprotein comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a sequence selected from the group consisting of SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82 and SEQ ID NO: 83.

In some embodiments, the present disclosure provides a nucleic acid encoding a preprotein comprising the amino acid sequence of SEQ ID NO:38. In some embodiments, the present disclosure provides a nucleic acid encoding a preprotein comprising the amino acid sequence of SEQ ID NO:39. In some embodiments, the present disclosure provides a nucleic acid encoding a preprotein comprising the amino acid sequence of SEQ ID NO:40. In some embodiments, the present disclosure provides a nucleic acid encoding a preprotein comprising the amino acid sequence of SEQ ID NO:41. In some embodiments, the present disclosure provides a nucleic acid encoding a preprotein comprising the amino acid sequence of SEQ ID NO:42. In some embodiments, the present disclosure provides a nucleic acid encoding a preprotein comprising the amino acid sequence of SEQ ID NO:43. In some embodiments, the present disclosure provides a nucleic acid encoding a preprotein comprising the amino acid sequence of SEQ ID NO:44. In some embodiments, the present disclosure provides a nucleic acid encoding a preprotein comprising the amino acid sequence of SEQ ID NO:45. In some embodiments, the present disclosure provides a nucleic acid encoding a preprotein comprising the amino acid sequence of SEQ ID NO:46. In some embodiments, the present disclosure provides a nucleic acid encoding a preprotein comprising the amino acid sequence of SEQ ID NO:47. In some embodiments, the present disclosure provides a nucleic acid encoding a preprotein comprising the amino acid sequence of SEQ ID NO:48. In some embodiments, the present disclosure provides a nucleic acid encoding a preprotein comprising the amino acid sequence of SEQ ID NO:49. In some embodiments, the present disclosure provides a nucleic acid encoding a preprotein comprising the amino acid sequence of SEQ ID NO:50. In some embodiments, the present disclosure provides a nucleic acid encoding a preprotein comprising the amino acid sequence of SEQ ID NO:51. In some embodiments, the present disclosure provides a nucleic acid encoding a preprotein comprising the amino acid sequence of SEQ ID NO:52. In some embodiments, the present disclosure provides a nucleic acid encoding a preprotein comprising the amino acid sequence of SEQ ID NO:53. In some embodiments, the present disclosure provides a nucleic acid encoding a preprotein comprising the amino acid sequence of SEQ ID NO:54. In some embodiments, the present disclosure provides a nucleic acid encoding a preprotein comprising the amino acid sequence of SEQ ID NO:55. In some embodiments, the present disclosure provides a nucleic acid encoding a preprotein comprising the amino acid sequence of SEQ ID NO:56. In some embodiments, the present disclosure provides a nucleic acid encoding a preprotein comprising the amino acid sequence of SEQ ID NO:57. In some embodiments, the present disclosure provides a nucleic acid encoding a preprotein comprising the amino acid sequence of SEQ ID NO:58. In some embodiments, the present disclosure provides a nucleic acid encoding a preprotein comprising the amino acid sequence of SEQ ID NO:59. In some embodiments, the present disclosure provides a nucleic acid encoding a preprotein comprising the amino acid sequence of SEQ ID NO:60. In some embodiments, the present disclosure provides a nucleic acid encoding a preprotein comprising the amino acid sequence of SEQ ID NO:61. In some embodiments, the present disclosure provides a nucleic acid encoding a preprotein comprising the amino acid sequence of SEQ ID NO:73. In some embodiments, the present disclosure provides a nucleic acid encoding a preprotein comprising the amino acid sequence of SEQ ID NO:74. In some embodiments, the present disclosure provides a nucleic acid encoding a preprotein comprising the amino acid sequence of SEQ ID NO:75. In some embodiments, the present disclosure provides a nucleic acid encoding a preprotein comprising the amino acid sequence of SEQ ID NO:76. In some embodiments, the present disclosure provides a nucleic acid encoding a preprotein comprising the amino acid sequence of SEQ ID NO:77. In some embodiments, the present disclosure provides a nucleic acid encoding a preprotein comprising the amino acid sequence of SEQ ID NO:78. In some embodiments, the present disclosure provides a nucleic acid encoding a preprotein comprising the amino acid sequence of SEQ ID NO:79. In some embodiments, the present disclosure provides a nucleic acid encoding a preprotein comprising the amino acid sequence of SEQ ID NO:80. In some embodiments, the present disclosure provides a nucleic acid encoding a preprotein comprising the amino acid sequence of SEQ ID NO:81. In some embodiments, the present disclosure provides a nucleic acid encoding a preprotein comprising the amino acid sequence of SEQ ID NO:82. In some embodiments, the present disclosure provides a nucleic acid encoding the preprotein comprising the amino acid sequence of SEQ ID NO:83.

In some embodiments, the present disclosure provides a nucleic acid comprising a sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a sequence selected from the group consisting of SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94 and SEQ ID NO:95.

In some embodiments, the present disclosure provides a nucleic acid comprising the sequence of SEQ ID NO:84. In some embodiments, the present disclosure provides a nucleic acid comprising the sequence of SEQ ID NO:85. In some embodiments, the present disclosure provides a nucleic acid comprising the sequence of SEQ ID NO:86. In some embodiments, the present disclosure provides a nucleic acid comprising the sequence of SEQ ID NO:87. In some embodiments, the present disclosure provides a nucleic acid comprising the sequence of SEQ ID NO:88. In some embodiments, the present disclosure provides a nucleic acid comprising the sequence of SEQ ID NO:89. In some embodiments, the present disclosure provides a nucleic acid comprising the sequence of SEQ ID NO:90. In some embodiments, the present disclosure provides a nucleic acid comprising the sequence of SEQ ID NO:91. In some embodiments, the present disclosure provides a nucleic acid comprising the sequence of SEQ ID NO:92. In some embodiments, the present disclosure provides a nucleic acid comprising the sequence of SEQ ID NO:93. In some embodiments, the present disclosure provides a nucleic acid comprising the sequence of SEQ ID NO:94. In some embodiments, the present disclosure provides a nucleic acid comprising the sequence of SEQ ID NO:95.

In some embodiments, the bispecific Fc fusion protein encoding nucleic acid as disclosed herein comprises a codon optimized version or variant.

“Codon optimized” in this context is done in relation to a particular host organism and its generally preferred amino acid codons; that is, the host production organism, e.g., an Aspergillus species, may yield higher translation and/or secretion using Aspergillus preferred codons as compared to a yeast production organism.

Codon optimization can be employed with any of the bispecific Fc fusion protein of the present disclosure, in order to optimize expression in the host cell employed.

The bispecific Fc fusion proteins comprising an IL-15 domain, an IL-15Rα sushi domain, an Fc domain and sPD-1 variant domain (short for “sPD-1 variant/IL-15 bispecific Fc fusion protein”) can be prepared generally by construction genes encoding the fusion protein sequence using well-known techniques, including site-directed mutagenesis of a parental gene and synthetic gene construction.

Expression of the nucleic acids of the present disclosure can be regulated by their own or by other regulatory sequences known in the art.

The present disclosure also relates to nucleic acid constructs comprising a polynucleotide encoding the sPD-1 variant/IL-15 bispecific Fc fusion protein of the present disclosure operably linked to one or more control sequences that direct the expression of the coding sequence in a suitable host cell under conditions compatible with the control sequences. The control sequence may include a promoter, a polynucleotide which is recognized by a host cell for expression of the polynucleotide. The promoter contains transcriptional control sequences that mediate the expression of the Fc fusion protein. The promoter may be any polynucleotide that shows transcriptional activity in the host cell including mutant, truncated, and hybrid promoters, and may be obtained from genes encoding extracellular or intracellular polypeptides either homologous or heterologous to the host cell.

In some embodiments, the control sequence may also be a signal peptide coding region that encodes a signal peptide linked to the N-terminus of the bispecific Fc fusion protein and directs the fusion protein being expressed into the cell's secretory pathway. The 5′-end of the coding sequence of the polynucleotide may inherently contain a signal peptide coding sequence naturally linked in translation reading frame with the segment of the coding sequence that encodes the bispecific Fc fusion protein. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the bispecific Fc fusion protein. However, any signal peptide coding sequence that directs the expressed fusion protein into the secretory pathway of a host cell may be used.

### F. Expression Vectors

Also provided herein are expression vectors for in vitro or in vivo expression of one or more sPD-1 variant/IL-15 bispecific Fc fusion proteins of the present disclosure, either constitutively or under one or more regulatory elements. The present disclosure provides expression vectors comprising any of the nucleic acid as disclosed herein. In some embodiments, the present disclosure relates to expression vectors comprising a polynucleotide encoding the sPD-1 variant/IL-15 bispecific Fc fusion protein, a promoter, and transcriptional and translational stop signals. The various nucleotide and control sequences may be joined together to produce a recombinant expression vector that may include one or more convenient restriction sites to allow for insertion or substitution of the polynucleotide encoding the bispecific Fc fusion protein at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector can be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used. Vectors contemplated for use with the methods of the disclosure include both integrating and non-integrating vectors.

### G. Host Cells and Production Strains

As will be appreciated by those in the art, there are a wide variety of production host organisms for the recombinant production of the sPD-1 variant/IL-15 bispecific Fc fusion protein of the present disclosure, including, but not limited to, bacterial cells, mammalian cells and fungal cells including yeast.

In some embodiments, the host cell comprises any of the nucleic acids as disclosed herein. In some embodiments, the host cell comprises any of the expression vectors as disclosed herein.

The nucleic acids of the disclosure can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

### H. Methods of Making the Fusion Proteins

The present disclosure also relates to methods of making an sPD-1 variant/IL-15 bispecific Fc fusion protein, comprising: (a) cultivating a host cell of the present disclosure under conditions suitable for expression of the sPD-1 variant/IL-15 bispecific Fc fusion protein; and (b) optionally recovering the sPD-1 variant/IL-15 bispecific Fc fusion protein.

### I. Method of Treatment

### 1. Subjects Amenable to Treatment

Various embodiments are directed to therapeutic methods, many of which include administering to a subject in need of treatment a therapeutically effective amount of one or more bispecific Fc fusion proteins as described herein.

A number of embodiments are directed to a method of treating, reducing or preventing metastasis or invasion of a tumor in a subject with cancer, the method comprising administering to the subject a therapeutically effective dose of one or more said bispecific Fc fusion proteins or said pharmaceutical composition as disclosed herein. In some embodiments, the tumor is a solid tumor. In some embodiments, the cancer is a colorectal cancer.

A number of embodiments are directed to a method of preventing or treating an infection in a subject, the method comprising administering to the subject a therapeutically effective dose of one or more said bispecific Fc fusion proteins or said pharmaceutical composition as disclosed herein. In some embodiments, the infection is selected from the group consisting of a fungal infection, bacterial infection and viral infection.

In some embodiments, the effective dose of the one or more bispecific Fc fusion proteins or the pharmaceutical composition used in the methods as disclosed herein inhibits, reduces, or modulates signal transduction mediated by the wild-type PD-1 in the subject.

In some embodiments, the effective dose of the one or more bispecific Fc fusion proteins or the pharmaceutical composition used in the methods as disclosed herein increases a T cell response in the subject.

A number of embodiments are directed to a method of preventing or treating an IL-15 mediated disease or disorder in a subject, the method comprising administering to the subject a therapeutically effective dose of said bispecific Fc fusion proteins or said pharmaceutical composition as disclosed herein, wherein the IL-15 mediated disease or disorder is a cancer or an infectious disease. In some embodiments, the cancer is colorectal cancer. In some embodiments, the infectious disease is a viral infection.

A number of embodiments are directed to a method of preventing or treating an immunodeficiency or lymphopenia in a subject, comprising administering to the subject a therapeutically effective dose of one or more said bispecific Fc fusion proteins or said pharmaceutical composition as disclosed herein.

A number of embodiments are directed to a method of enhancing IL-15-mediated immune function in a subject in need thereof, comprising administering to the subject a therapeutically effective dose of one or more said bispecific Fc fusion proteins or said pharmaceutical composition as disclosed herein. In some embodiments, the enhanced IL-15-mediated immune function comprises proliferation of lymphocytes, inhibition of apoptosis of lymphocytes, antibody production, activation of antigen presenting cells and/or antigen presentation. In some embodiments, the enhanced IL-15-mediated immune function comprises activation or proliferation of CD4+ T cells, CD8+ T cells, B cells, memory T cells, memory B cells, dendritic cells, other antigen presenting cells, macrophages, mast cells, natural killer T cells (NKT cells), tumor-resident T cells, CD122+ T cells, and/or natural killer cells (NK cells).

A number of embodiments are directed to a method of promoting T cell cytotoxicity or NK cell cytotoxicity in a subject in need thereof, comprising administering to the subject a therapeutically effective dose of one or more said bispecific Fc fusion proteins or said pharmaceutical composition as disclosed herein.

### 2. Therapeutic Administration

In certain embodiments, a therapeutically effective composition or formulation comprising one or more bispecific Fc fusion proteins of the present disclosure may be administered systemically to the individual in need thereof or via any other route of administration known in the art.

### 3. Dosing

In some embodiments, an effective dose of the therapeutic entity of the present disclosure, e.g., for the treatment of cancers or infections, varies depending upon many different factors, including means of administration, target site, physiological state of the patient, whether the patient is human or an animal, other medications administered, and whether treatment is prophylactic or therapeutic. Treatment dosages can be titrated to optimize safety and efficacy.

### VI. Examples

### A. Example 1: Cell Line Development and Clone Selection

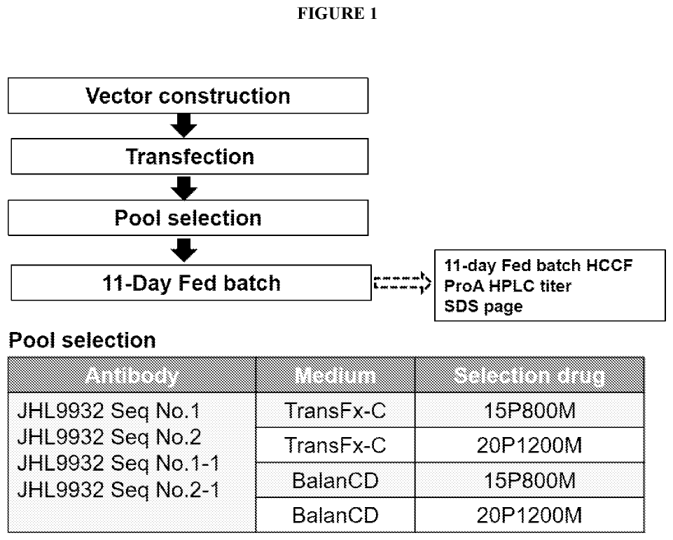

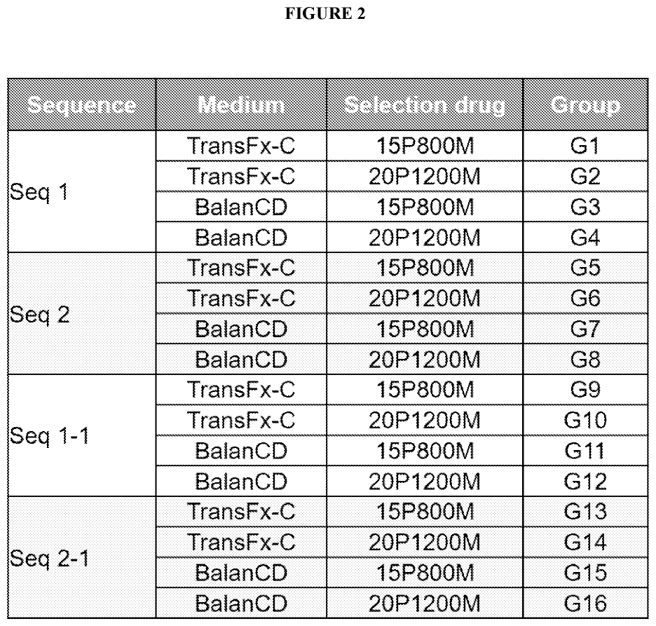

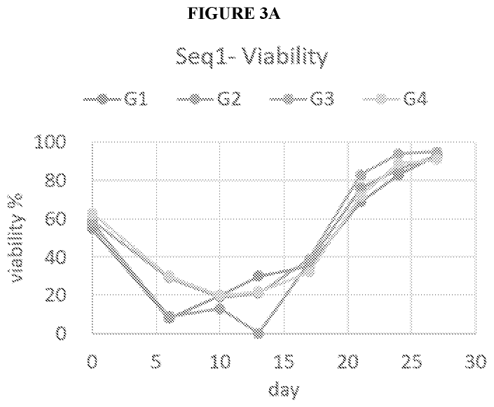

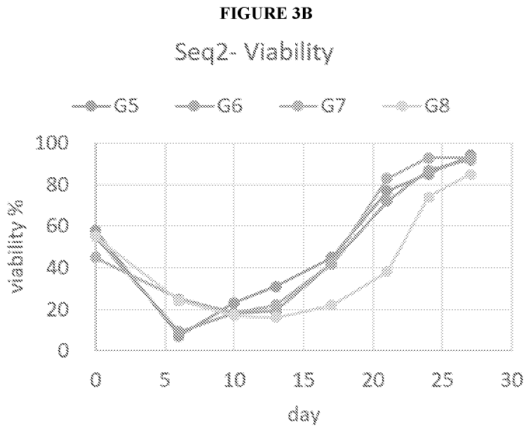

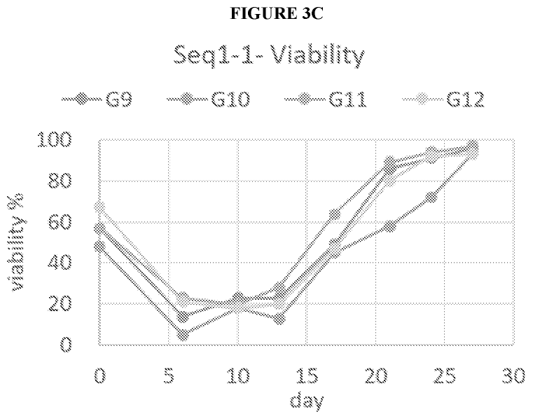

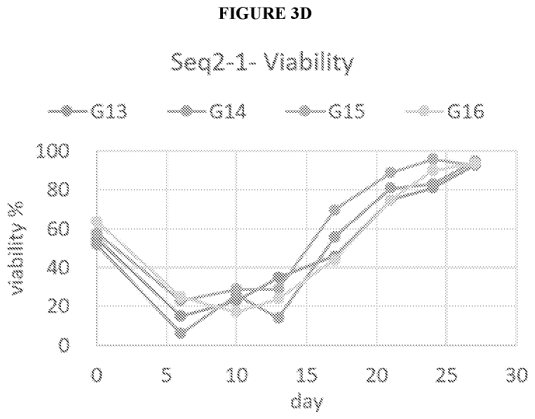

CHO-K1-C6-4G5 host cells maintained in exponential phase with HyCell TranFx-C medium for three passages. On the day of transfection, cells were adjusted to viable cell density 1E+06 cells/mL in 25 mL cell culture (125 mL shake flask). Preparation of the following transfection mixtures: 50 μL of FreeStyle MAX was diluted in 1.5 mL OptiPRO SFM and incubated at room temperature for 3˜5 minutes. 50 gg of linearized expression plasmid pJHL-Aldoa-PuroRs-JHL9932 and pJHL-Aldoa-DHFRs-JHL9932 was diluted in 1.5 mL OptiPRO SFM. The FreeStyle MAX solution was then mixed with the DNA solution and leave at room temperature for 15 minutes. After incubation, the solution was added into the CHO-K1-C6-4G5 culture (25 mL in a 125 mL shake flask). Transfected cells were incubated in a 130 rpm, 37° C., 5% CO₂ incubator. One portion of transfected cells were used for stable pools generation 24 hours post-transfection. 48 hours post-transfection, transfected cells were subjected to drug selection. Cells were seeded at density of 4˜5 E+05 cells/mL in 15˜20 mL medium in a 150T Flask with selection drug (15 or 20 Q g/ml Puromycin and 800 or 1200 nM MTX). As shown in the flow chart, two media were applied. One is HyCell TransFx-C containing 4 mM L-glutamine and 0.1% F-68, another is BalanCD CHO Growth A containing 4 mM L-glutamine (FIG. 1). Once cell viability achieved more than 50%, cells would be expanded to 10 mL culture in 50 mL spintube for pool recovery (FIG. 2). In another 5 to 10 days, each pool would recovery to about 90% viability. Viability data showed that all 16 pool had successfully recovered to 90% (FIG. 3A-D). Cell would be expanded to 25 mL culture in a 125 mL shake flask, incubated in a 130 rpm, 37° C., 5% CO₂ incubator. Once each pool reached 90% viability by the following subculture, cryopreservation was performed for at 3 vials per pool.

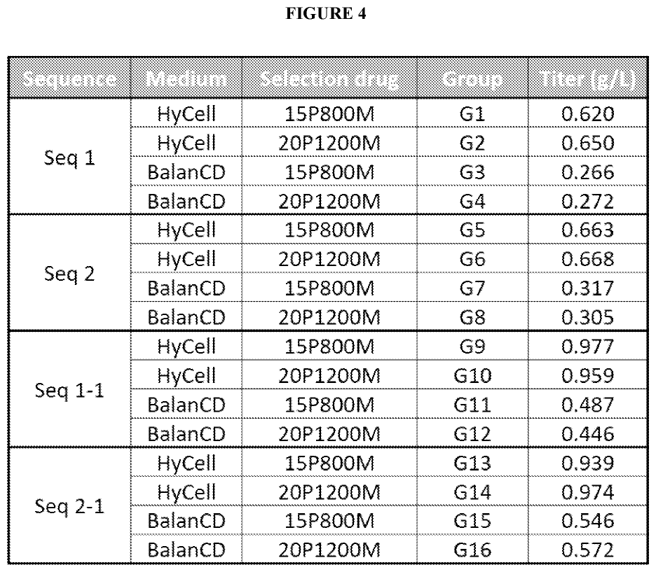

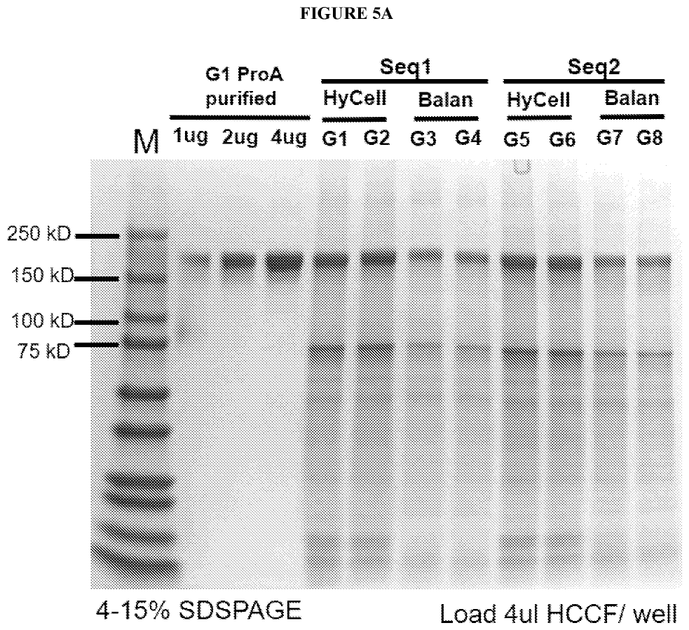

Cell culture fluids were harvested on day 11 and followed by centrifugation at 3000 g, 15 minutes, 22° C. The culture supernatant was passed through a 0.22 m filter and the titer is determination using (ProA-HPLC). Specifically, pools cultured in HyCell TransFx-C Medium produced better titer than pools cultured with BalanCD CHO Medium. G9, G10, G13 and G14 produced the highest titer (FIG. 4). A portion of filtered supernatants was purified by Protein A HP Spin Trap was subjected to in-House SDS-PAGE and in-House developed Bioassay PD-L1, PDL-2 and IL15R-beta binding (ELISA). The relative binding potency assay result of PD-L1, PDL-2 and IL15R-beta gave more than 100% versus pool G9 as reference standard which is set 100%.

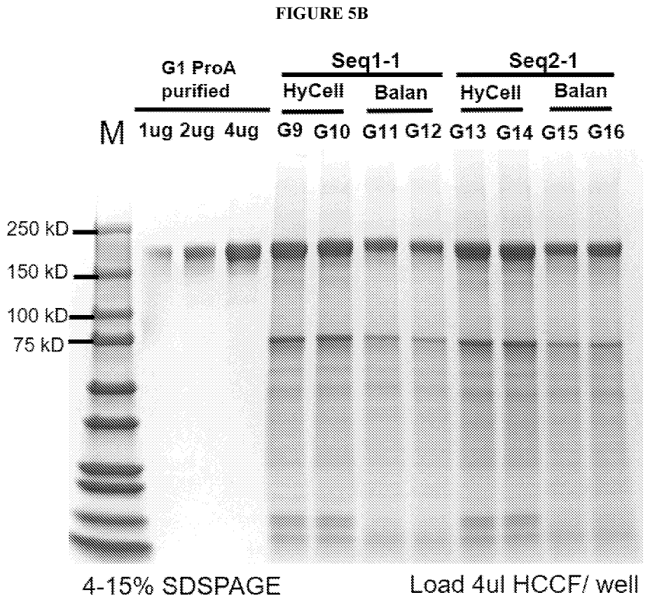

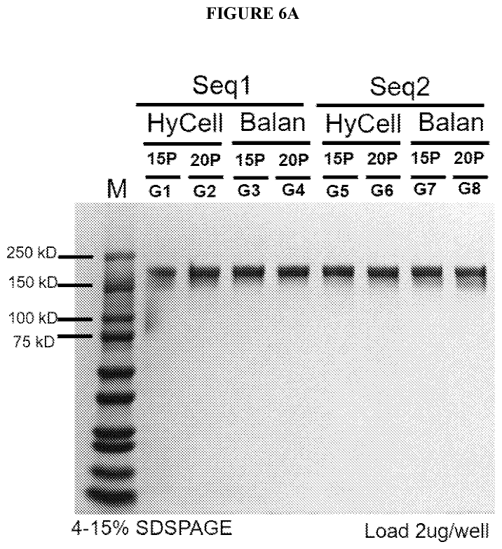

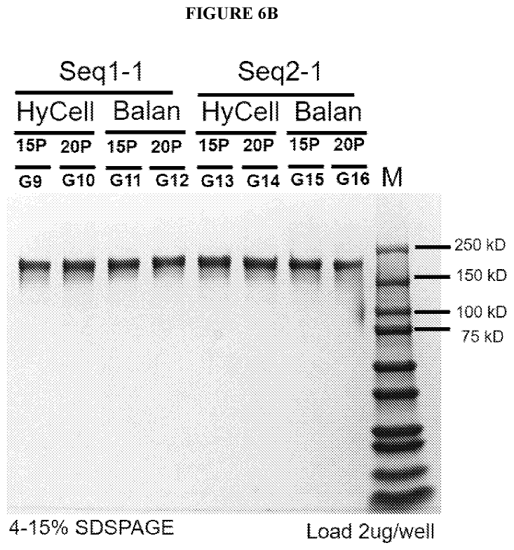

Recovered Phase I pools by 11 days fed-batch culture were subject to SDS-PAGE analysis on both Harvested Cell Culture Fluid (HCCF) (FIG. 5A, B) and ProA purified samples (FIG. 6A, B). 4 ul of HCCF and 2 ug of ProA purified samples were ran on 4-15% SDS-PAGE. The major band of target is approximately 150 kDa and 200 kDa. All clones had a visible major band around 150 kDa and 200 kDa.

### B. Example 2: ELISA Potency Assays

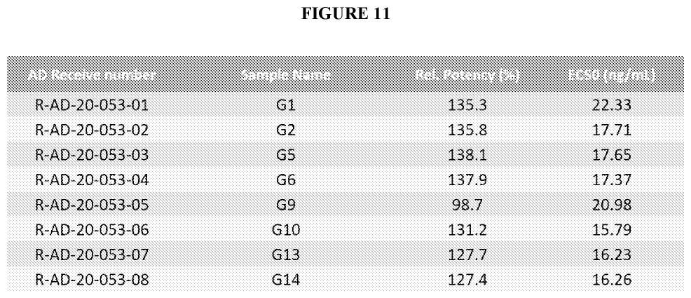

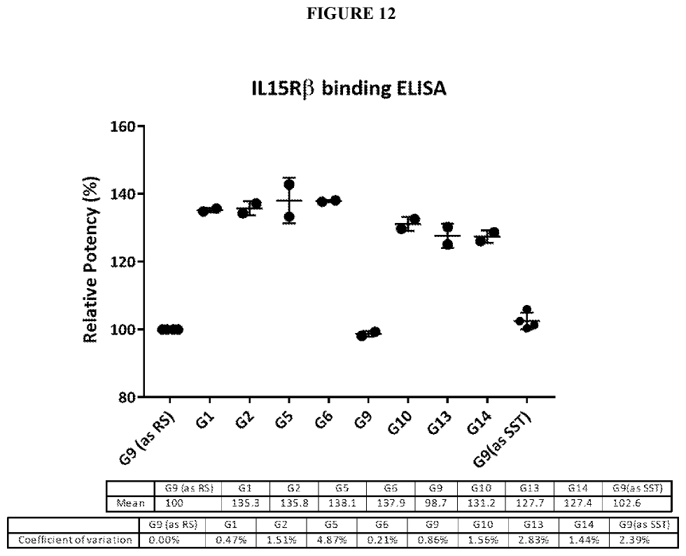

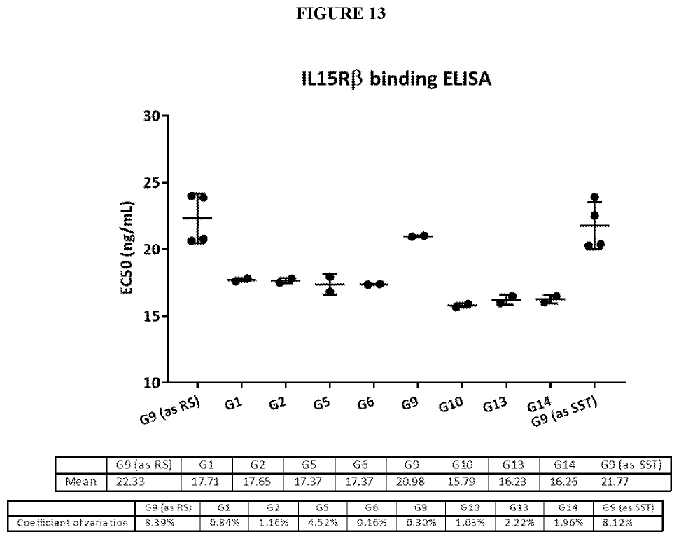

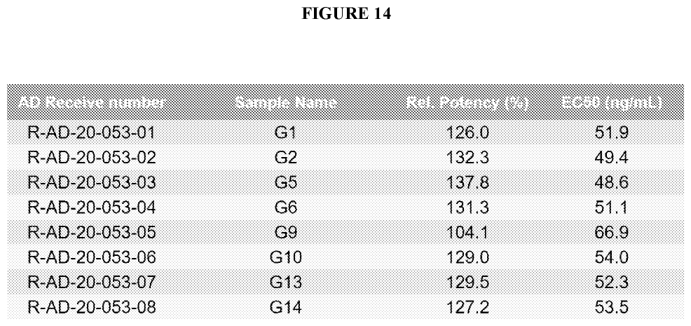

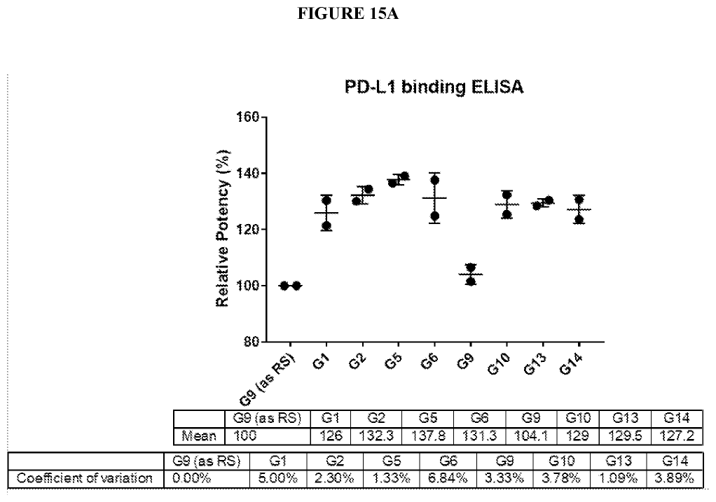

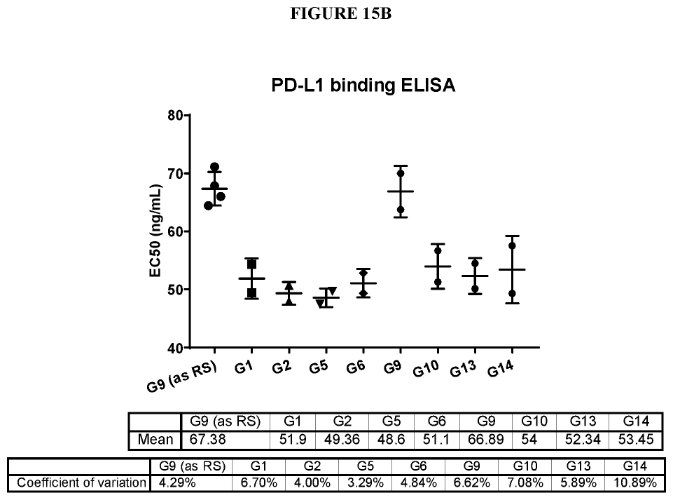

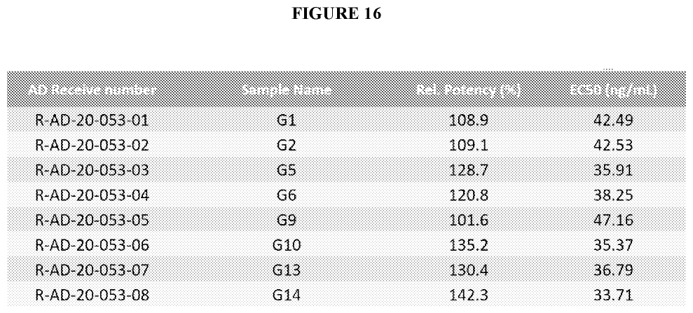

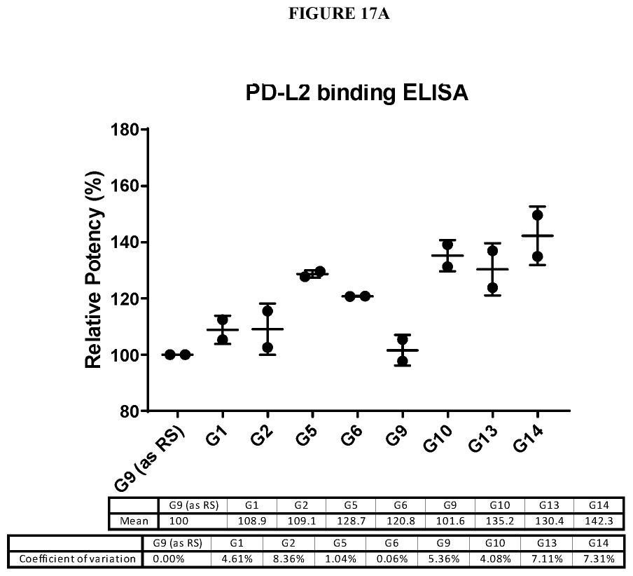

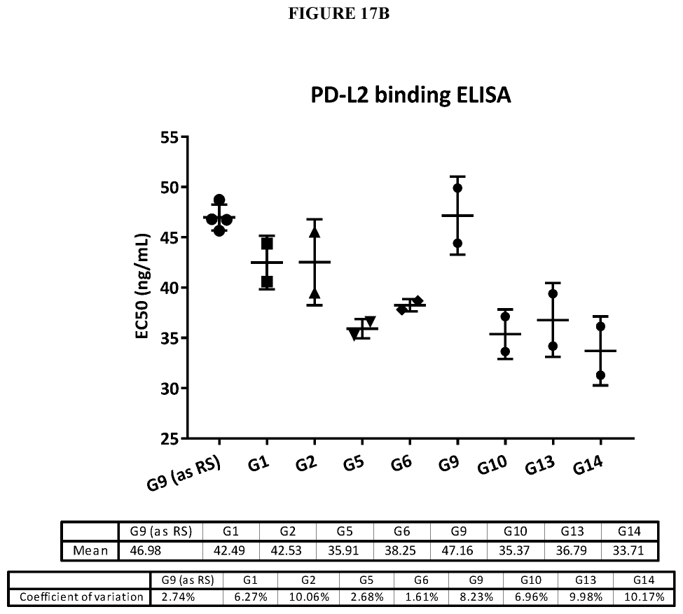

The PD-L1, PD-L2 and IL-15R-beta binding potency of pooled materials were analyzed using in-house developed ELISA based Bioassay for PD-L1, PD-L2 and IL15R-beta binding. Pool G9 was used as Reference Standard (RS) and System Suitability Test (SST) and set to 100%. The tabulated summaries of pooled clones binding to IL-15 is shown in FIG. 11, and results demonstrating IL-15 potency are shown in FIG. 12 and FIG. 13. Specifically, pool G1, G2, G5, G6, G10, G13 and G14 all showed higher potency and lower EC50 than G9 reference standard in the IL-15 ELISA binding study. Tabulated summaries of pooled clones binding to PD-L1 is shown in FIG. 14, and results demonstrating PD-L1 potency are shown in FIG. 15A and EC50 in FIG. 15B. Pool GI, G2, G5, G6, G10, G13 and G14 all showed higher potency and lower EC50 than G9 reference standard in the PD-L1 ELISA binding study with pool G5 showing highest potency and EC50. Tabulated summaries of pooled clones binding to PD-L2 is shown in FIG. 16, and results demonstrating PD-L2 potency are shown in FIG. 17A and FIG. 17B. Pool G5, G10, G13 and G14 has the high potency and low EC50 to PD-L2 binding.

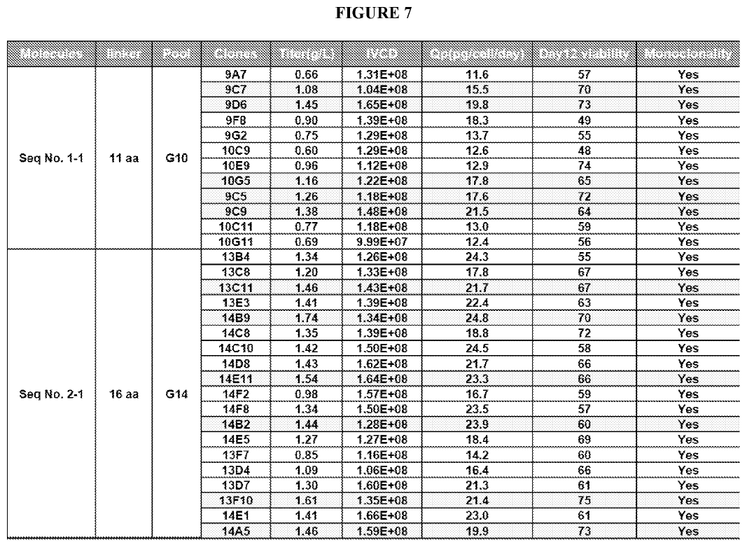

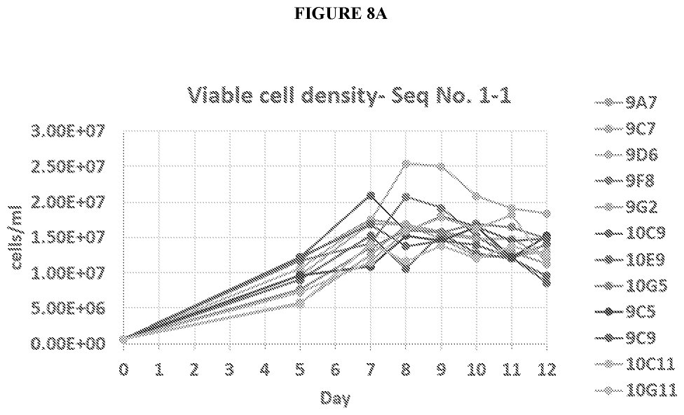

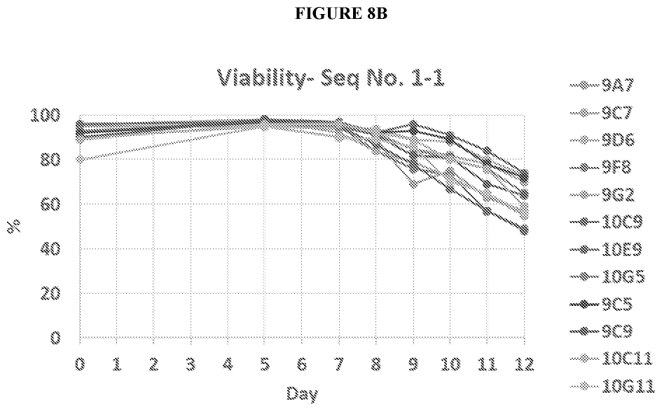

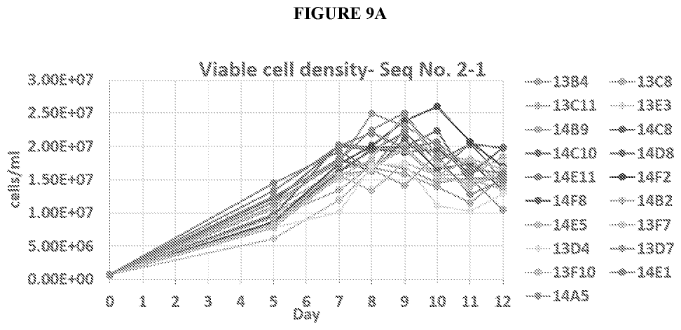

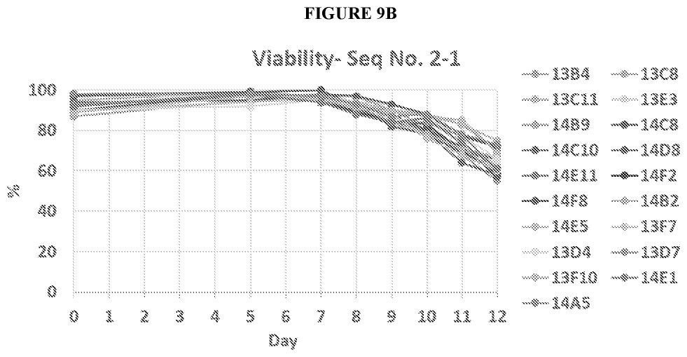

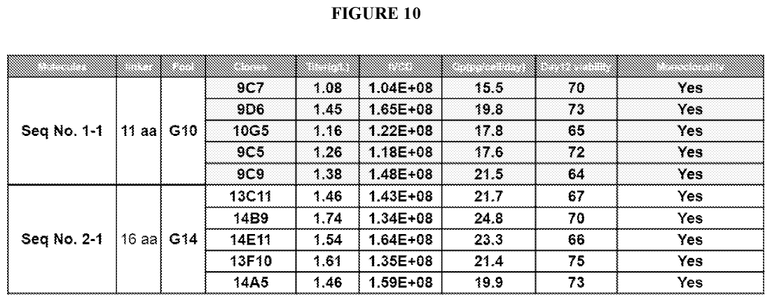

Pooled clones G10 and G14 were picked for phase II pool selection and single cell cloning was performed. There were 112 clones picked from the 96-well plates and expanded with selection drug contained medium (60 μg/mL puromycin+3000 nM MTX). Some clones would not be able to survive under selection drugs gradually increasing back to 100% during a scale-up process. Ultimately the clone would be transferred from a 6 well plate into a 5˜6 mL culture in a 50 mL Spin tube, incubated in a 180 rpm, 37° C., 5% CO₂ incubator. Clones that were able to grow in a 50 mL Spin tube and eventually achieved >=90% viability and the viable cell density >=1E+06 cells/mL would be cryopreserved (FIGS. 8A, B and FIGS. 9A, B). There were 31 clones cryopreserved and proceeded to fed-batch culture for clone evaluation (FIG. 7). Finally, top 10 single cell clones were selected based on titer, viability and monoclonality, 5 clones from pool G10 and 5 clones from pool G14 (FIG. 10).

### C. Example 3: In Vivo Validation

Anti-tumor activity of sPD-1 variant/IL-15 bispecific (or bifunctional) Fc fusion protein (SEQ ID NO: 97) was analyzed and compared to the anti-tumor activities of sPD-1 variant and IL-15 molecules alone or in combination. One group of mice received saline as a vehicle control group, and six groups of mice received the following treatments respectively via intraperitoneal (IP) injection.

Group 1 is sPD-1 variant single treatment comprised of sPD-1 variant and hIgG4 Fc domain at 10 mg/kg.

Group 2 is IL-15 single treatment comprised of IL-15, IL-15 sushi domain and hIgG4 at 2.5 mg/kg.

Group 3 is sPD-1 variant/IL-15 bifunctional Fc fusion protein treatment (short for “bifunctional treatment”) at 0.1 mg/kg.

Group 4 is sPD-1 variant+IL-15 combined treatment (short for “combined treatment”) comprised of sPD-1 variant single molecule at 10 mg/kg plus IL-15 single treatment comprised of IL-15, IL-15 sushi domain and hIgG4 at 2.5 mg/kg.

Group 5 is bifunctional treatment at 1 mg/kg.

Group 6 is bifunctional treatment at 10 mg/kg.

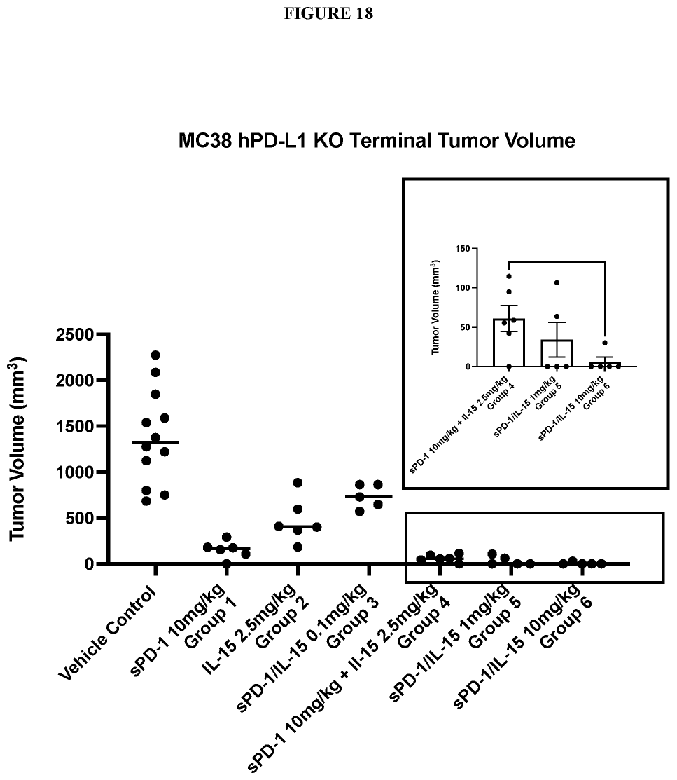

Final tumor volume of MC38-hPD-L1 colorectal tumors were compared among tumors after receiving treatments of vehicle control, Group 1, Group 2, Group 3, Group 4, Group 5 and Group 6. (FIG. 18). Inlaid graph of FIG. 18 shows the results on volumes of the tumors after receiving combined treatment (Group 4) and the bifunctional treatment at 1 mg/kg (Group 5) and 10 mg/kg (Group 6). The treatment with sPD-1 variant/IL-15 bifunctional Fc fusion protein (10 mg/kg) (Group 6) showed significant and synergistic effects on decreasing the tumor volume when compared to the sPD-1 variant+IL-15 combined treatment (Group 4). The detailed calculation is shown below:

### sPD-1/IL-15 Bifunctional Fc Fusion Protein Treatment (or “Bifunctional Treatment”)

Bivalent whole molecule size: 67.36×2=134.72 kDa (excluding post translation modification)

IL-15 domain molecular size=47.8 kDa (which is 35.4% of the total molecule). For every 1 mg/kg of the bifunctional treatment, 0.354 mg/kg of IL-15 was injected into mice.

sPD-1 variant domain molecular size=32.7 kDa (which is 24% of the molecule). For every 1 mg/kg of the bifunctional treatment, 0.24 mg/kg of sPD-1 variant was injected into mice.

IL-15 single treatment comprised of IL-15, IL-15 sushi domain and hIgG4 at 2.5 mg/kg;

Bivalent molecule size: 100.2 kDa (excluding post translation modification)

IL-15=47.8 kDa (which is 47.7% of the molecule). For every 1 mg/kg of the IL-15 single treatment, 0.477 mg/kg of the IL-15 was injected into mice.

sPD-1 Variant Single Treatment:

Bi-valent whole molecule size: 90.34 kDa (excluding post translation modification)

sPD-1 variant=38.46 kDa (which is 42.5% of the molecule). For example, for every 1 mg/kg of the sPD-1 variant single treatment, there was 0.425 mg/kg of the sPD-1 variant injected into mice.

### In Summary:

If dosed with 10 mg/kg of sPD-1 variant single treatment (Group 1), 4.25 mg/kg of sPD-1 variant was injected into mice.

If dosed with 2.5 mg of IL-15 single treatment (Group 2), 1.19 mg/kg of IL-15 was injected into mice.

If dosed with 0.1 mg/kg of sPD-1 variant/IL-15 bifunctional Fc fusion protein treatment (Group 3), 0.0354 mg/kg of IL-15 and 0.024 mg/kg of sPD-1 variant were injected into mice.

If dosed with sPD-1 variant+IL-15 combined treatment (Group 4) as disclosed above, 4.25 mg/kg of sPD-1 variant and 1.19 mg/kg of IL-15 were injected into the mice.

If dosed with 1 mg/kg of sPD-1 variant/IL-15 bifunctional Fc fusion protein treatment (Group 5), 0.354 mg/kg of IL-15 and 0.24 mg/kg of sPD-1 variant were injected into mice.

If dosed with 10 mg/kg of sPD-1 variant/IL-15 bifunctional Fc fusion protein treatment (Group 6), 3.54 mg/kg of IL-15 and 2.4 mg/kg of sPD-1 variant were injected into mice.

The above calculations show that Group 5 (bifunctional treatment at 1 mg/kg) and Group 4 (combined treatment) demonstrate similar if not comparable post-treatment tumor volume, however, there are much less sPD-1 variant and IL-15 injected into the animal's body for Group 5 (0.354 mg/kg IL-15 and 0.24 mg/kg sPD-1 variant) than those for Group 4 (1.19 mg/kg IL-15 and 4.25 mg/kg sPD-1 variant). Therefore, assuming Group 4 (combined treatment) and Group 5 (bifunctional treatment at 1 mg/kg with SEQ ID NO: 97) showing similar post-treatment tumor volumes, Group 5 uses 3.4x less IL-15 and almost 17.7x less sPD-1 variant than Group 4, revealing that the sPD-1 variant/IL-15 bifunctional Fc fusion protein (SEQ ID NO: 97) treatment has significantly higher anti-tumor efficacy than the sPD-1 variant+IL-15 combined treatment.

### D. Example 4: In Vivo Study Comparing the Anti-Tumor Activities of AB002 and Various PD-1 Immune Checkpoint Inhibitors in Combination with 11-15 Agonist

Anti-tumor activity of AB002, sPD-1/IL-15 bifunctional Fc fusion protein (SEQ ID NO: 97), was analyzed and compared to the anti-tumor activities of aPD-1, aPD-L1, and IL-15 molecules alone or in combination.

### Tumor Inoculation and Treatment Protocol

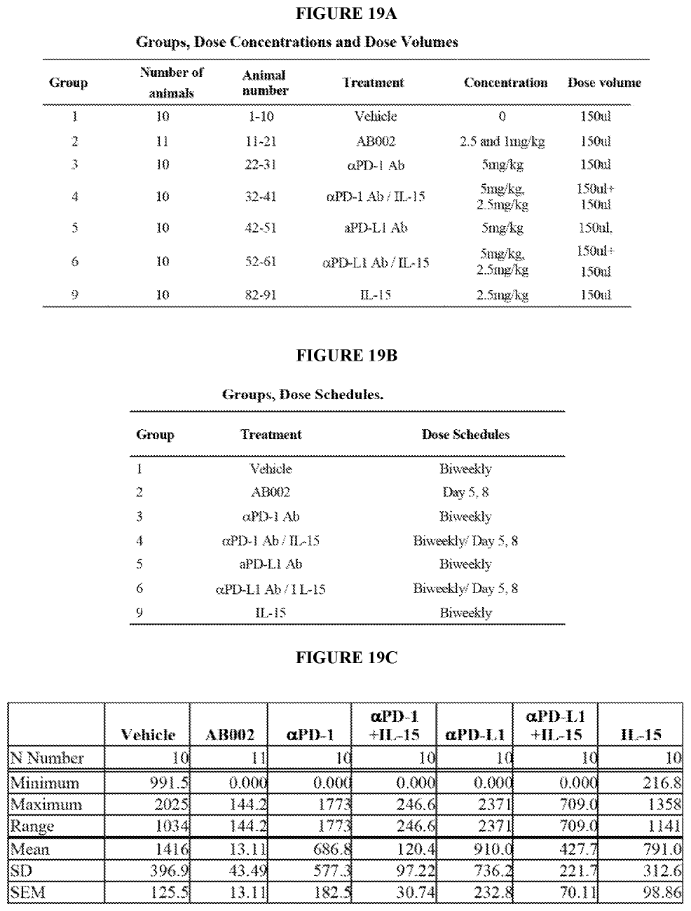

Female C57BL/6 mice were injected with 3×10⁶ MC38 tumor cells subcutaneously under isoflurane inhalation anesthesia. Mice were monitored for signs of distress up to 24 hours post injection. One group of mice received saline as a vehicle control group, and six groups of mice received treatments via intraperitoneal (IP) injection for 15 days. Treatment groups, dose concentration, and dose volume used in the treatment are listed in FIG. 19A. Dosing schedule is listed in FIG. 19B.

### Tumor Growth Monitoring

For each treatment group, treatment initiated when tumors became palpable at day 5. Tumor growths were monitored throughout the study by measuring the width, length and height of tumor using a digital caliper. Tumor volume (mm³) were calculated using the equation:

$$Tumor Volume = π / 6 * width * length * height$$

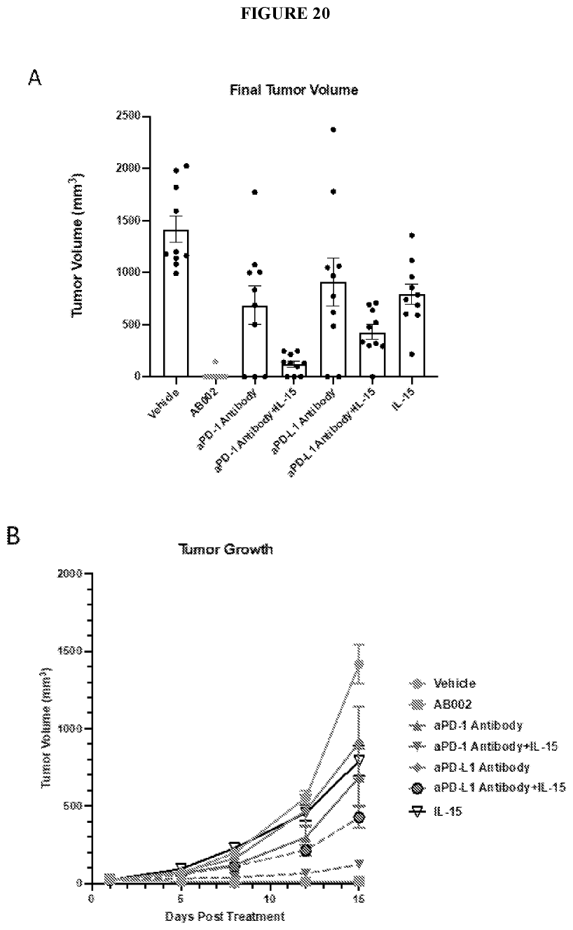

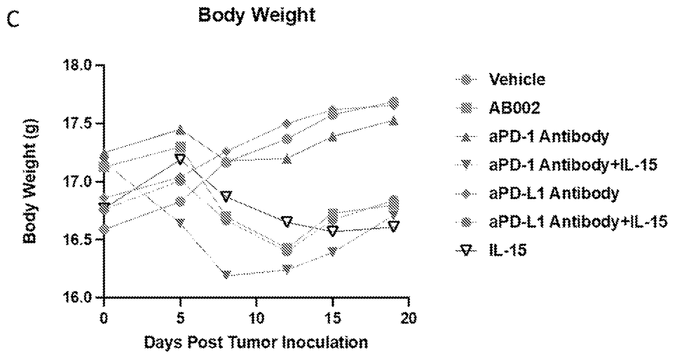

Tumor growth curves were generated at the end of the study. FIG. 20A shows the final tumor volume. FIG. 20B shows the overall tumor growth in animals treated. AB002 treated animals showed significant reduction in tumor growth and AB002 treatment resulted in complete cure in 10 out of 11 treated animals. When aPD-1 and aPD-L1 were used in combination with IL-15, the combination groups consistently showed enhanced antitumor activity compared to aPD-1, aPD-L1 and IL-15 used alone as shown in FIG. 19C.

### Animal Toxicity

Animal weights were recorded throughout the study period. A reduction in total body weight was observed in AB002, aPD-1 Ab/IL-15, aPD-L1 Ab/IL-15 and IL-15 treated groups. AB002-treated mice showed initial reduction in body weight but was able to regain most of the body weight after treatment as shown in FIG. 20C. This was consistent with IL-15 associated weight reduction previously reported.

### Conclusion

AB002 showed a significant anti-tumor activity when used as a standalone agent for treating MC38 colorectal cancer in vivo. AB002 demonstrates a superior anti-tumor activity when used in head-to-head comparison with other PD-1 immune checkpoint inhibitors used alone or in combination with IL-15.

### E. Example 5: RNA Sequencing Profiling Comparing MC38 Tumors Treated with AB002 vs aPD-1 Antibody

MC38 tumors treated with AB002 (2.5 mg/kg; SEQ ID NO: 52) (N=6) or mouse aPD-1 antibody (10 mg/kg) (N=6) were harvested for RNA sequencing analysis, focusing on immune oncology panel. The volcano plot in FIG. 21 show that 46 genes were identified as target genes showing significant change in their expression (|Fold Change|>2, p-value <0.05). Red dots represent 15 genes that were upregulated (Cx3cr1, Lilra5, Rtn1, Npl, P2ry13, Col4a5, Snca, Selenop, Hbb-bs, Hbb-bt, Col5al, Hba-a1, Hba-a2, Spib, and Fcrls) and blue dots represent the other 31 genes that were downregulated.

### Conclusion

Compared to aPD-1 Ab treatment, MC38 tumors treated with SEQ ID NO: 52 can uniquely downregulates Ccl3 and Ccl4 mRNA transcripts, which are chemokine ligands of CCR5 for promoting pro-tumor CCR5⁺ MDSC infiltration. In addition, AB002 (SEQ ID NO: 52) treatment also leads to decreased NK and T cell exhaustion markers Cd274 (Pd-L1) and Tigit, suggesting that AB002 treatment may reverse MDSC infiltration and inhibits NK and T cell exhaustion phenotypes.

### F. Example 6: AB002 Demonstrated Superior Tumor Inhibition Compared to aPD-1 Antibody in Immunotherapy-Resistant Lewis Lung Tumor Models

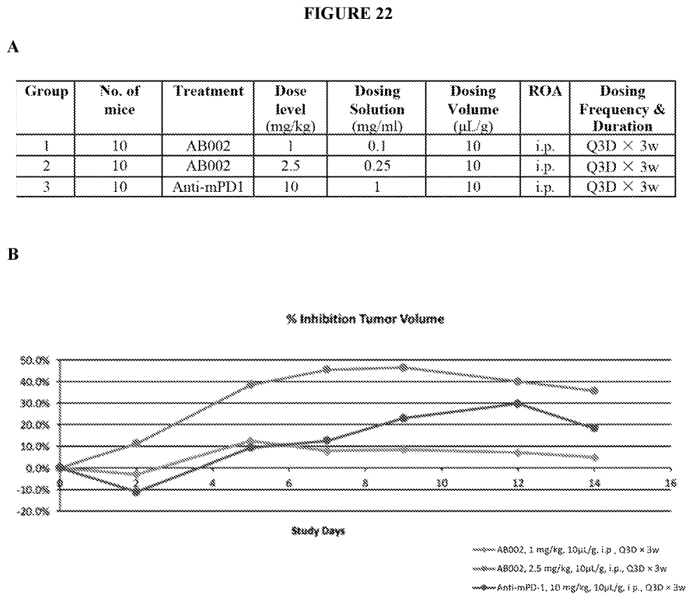

Female C57BL/6 mice were injected subcutaneously with 3×10⁵ Lewis Lung Carcinoma tumor cells under isoflurane inhalation anesthesia. Mice were monitored for signs of distress up to 24 hours post injection. Treatment groups, dose concentration and dose schedule are listed in FIG. 22 item A. Treatment group randomization started when the mean tumor size reached approximately 100 mm³. 50 mice were enrolled in the study. All animals were randomly allocated to 5 study groups with 10 mice in each group. Randomization was performed based on “Matched distribution” method/“Stratified” method (StudyDirector™ software, version 3.1.399.19) /randomized block design. The date of randomization was denoted as day 0.

After tumor cell inoculation, the animals were checked daily for morbidity and mortality. During routine monitoring, the animals were checked for any effects on tumor growth and treatments on behavior such as mobility, food and water consumption, body weight change (Body weight was measured 3 times per week after randomization), eye/hair matting and any other abnormalities. Mortality and observed clinical signs were recorded for individual animals in detail.

The treatment with AB002 (SEQ ID NO: 52) was initiated on the same day of randomization (day 0) per study design. Tumor volumes was measured 3 times per week after randomization in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). The body weights and tumor volumes were measured by using StudyDirector™ software (version 3.1.399.19). Percent inhibition of Tumor volume following treatment is presented in FIG. 22 item B. Dosing as well as tumor and body weight measurements were conducted in a Laminar Flow Cabinet. Results at day 14 as shown in FIG. 22 item B show highest level of tumor inhibition by 2.5 mg/kg of AB002 (SEQ ID NO: 52), followed by anti-mPD-1 at 10 ml/kg.

In sum, AB002 demonstrated superior anti-tumor activity used in head-to-head comparison with PD-1 immune checkpoint inhibitors as a single agent in Lewis Lung Tumor models.

### G. Example 7: Relationship Between Treatment Dosing and Therapeutic Efficacy of AB002 in MC38 Tumor Bearing Mice

Female C57BL/6 mice were injected subcutaneously with 5×10⁶ MC38 tumor cells suspended in 50% Matrigel under isoflurane inhalation anesthesia. Mice were monitored for signs of distress up to 24 hours post injection. Mice were randomized into treatment groups once tumor reached approximately 200 mm³ around day 10 following tumor inoculation. Five female mice were assigned per each of the 15 treatment groups with or without AB002 (SEQ ID NO: 97): 1) Vehicle Control; 2) intravenous administration (“IV”) 0.5 mg/kg of AB002; 3) IV 1 mg/kg of AB002; 4) IV 2.5 mg/kg of AB002; 5) IV 5 mg/kg of AB002; 6) Subcutaneous administration (“SQ”) 0.5 mg/kg of AB002; 7) SQ 1 mg/kg of AB002; 8) SQ 2.5 mg/kg of AB002; 9) SQ 5 mg/kg of AB002; 10) IV 0.67 mg/kg of sPD-1, equivalent to 1 mg/kg AB002; 11) IV 1.675 mg/kg of sPD-1, equivalent to 2.5 mg/kg AB002; 12) IV 3.35 mg/kg of sPD-1, equivalent to 5 mg/kg AB002; 13) 1 mg/kg of AB002+5 mg/kg of sPD-1; 14) 1 mg/kg of AB002+5 mg/kg of aPD-1 Ab (pembrolizumab); 15) 1 mg/kg of AB002+5 mg/kg of aPD-L1 Ab (atezolizumab). All treatments were given once weekly. The animals were checked daily for morbidity and mortality. During routine monitoring, the animals were checked for any effects of tumor growth and treatments on behavior such as mobility, food and water consumption, body weight change (Body weights were measured 3 times per week after randomization), eye/hair matting and any other abnormalities. Mortality and observed clinical signs were recorded for individual animals in detail. Tumor growths were monitored throughout the study by measuring the width, length, and height of tumor using a digital caliper. Tumor volume (mm³) were calculated using the equation:

$$Tumor Volume = π / 6 * width * length * height$$

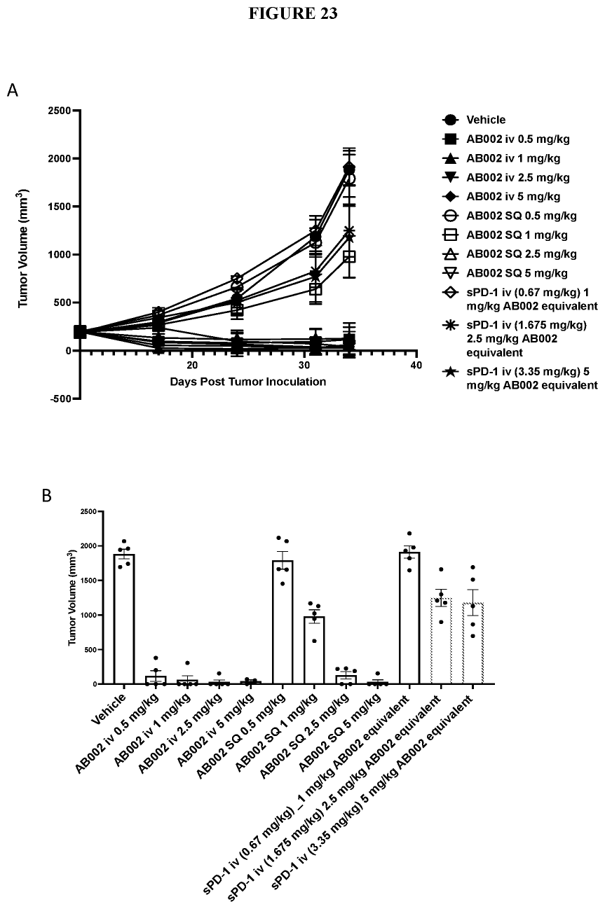

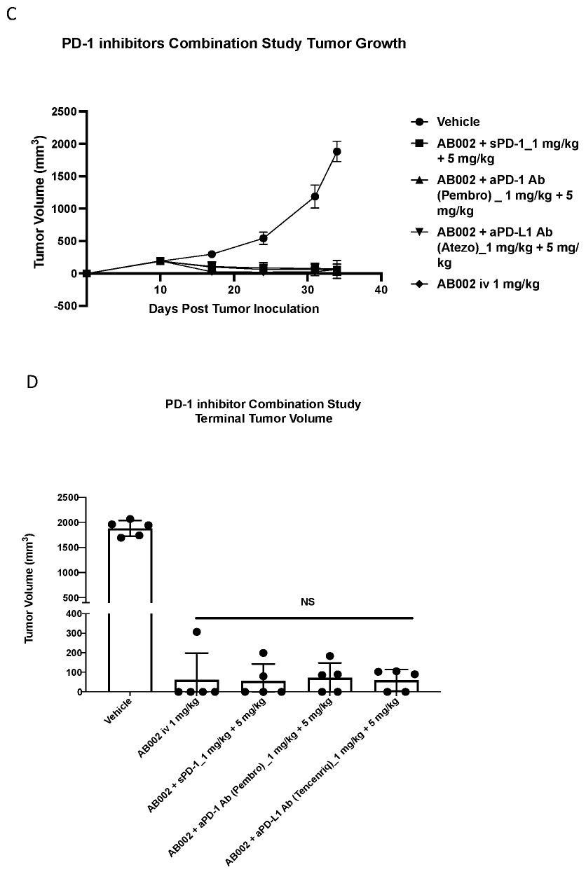

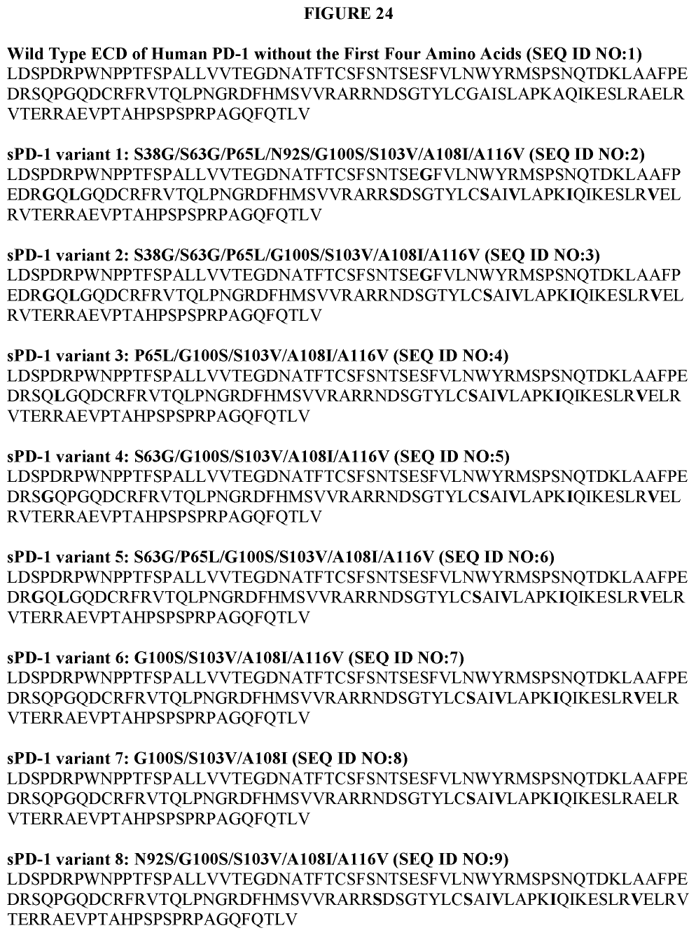

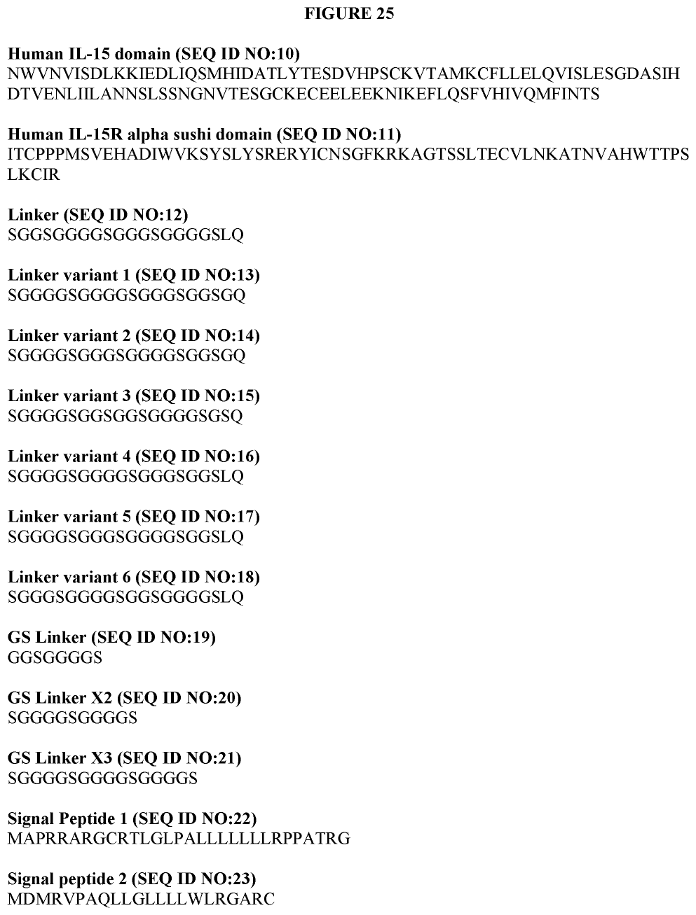

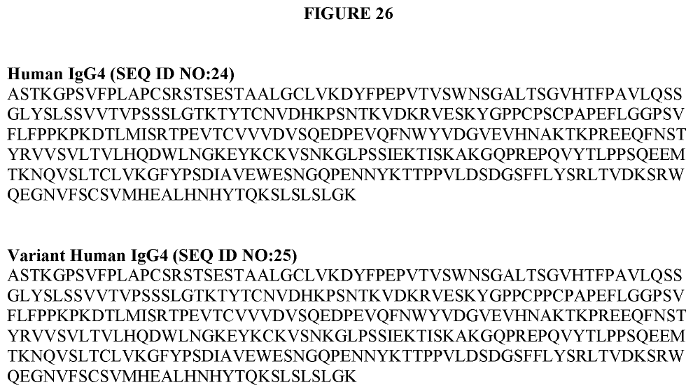

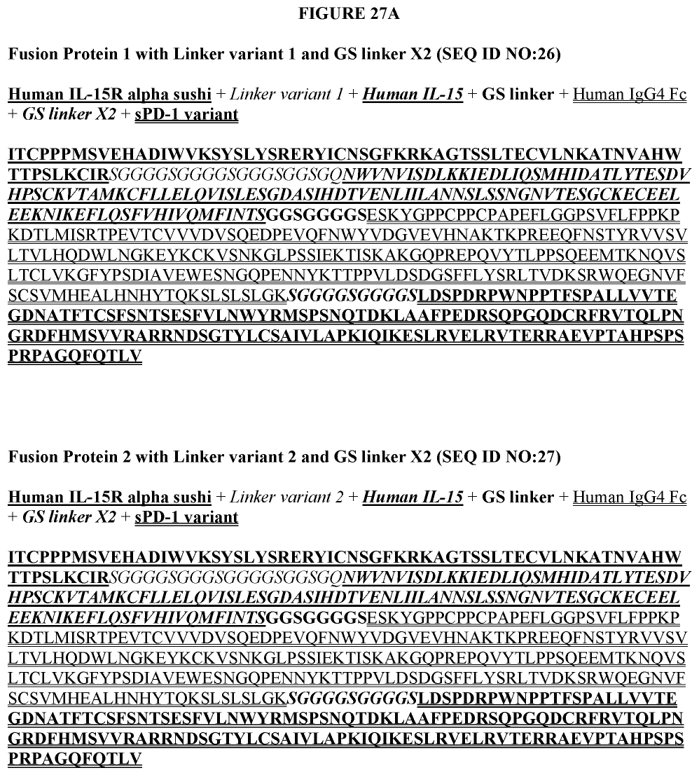

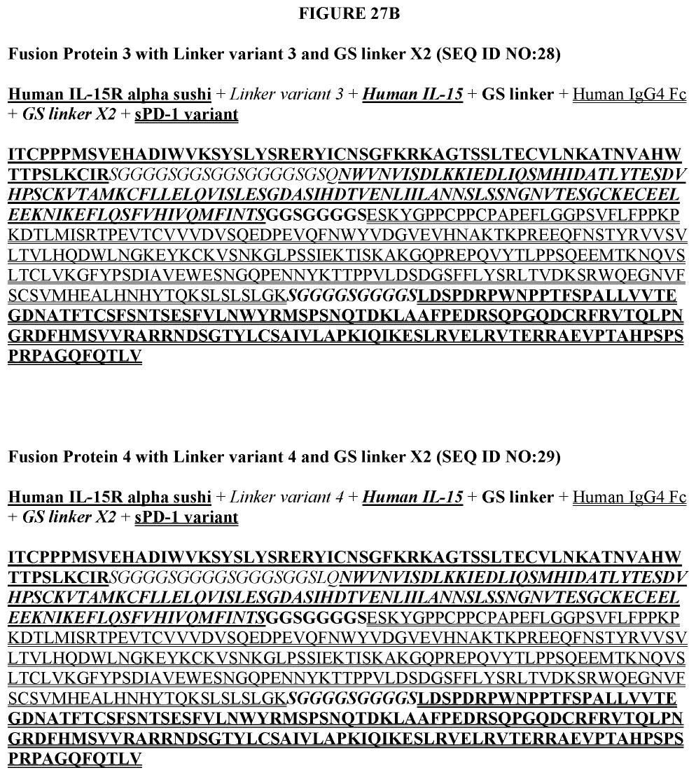

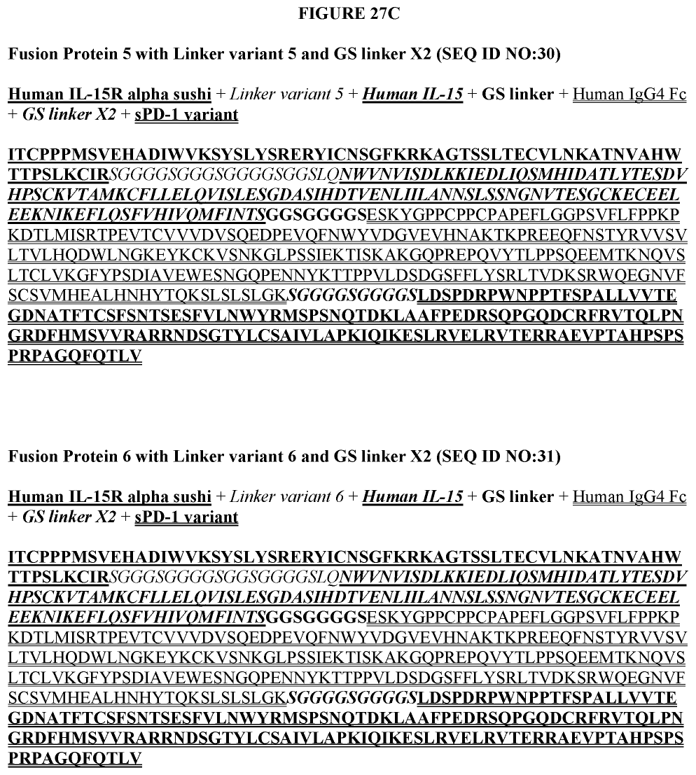

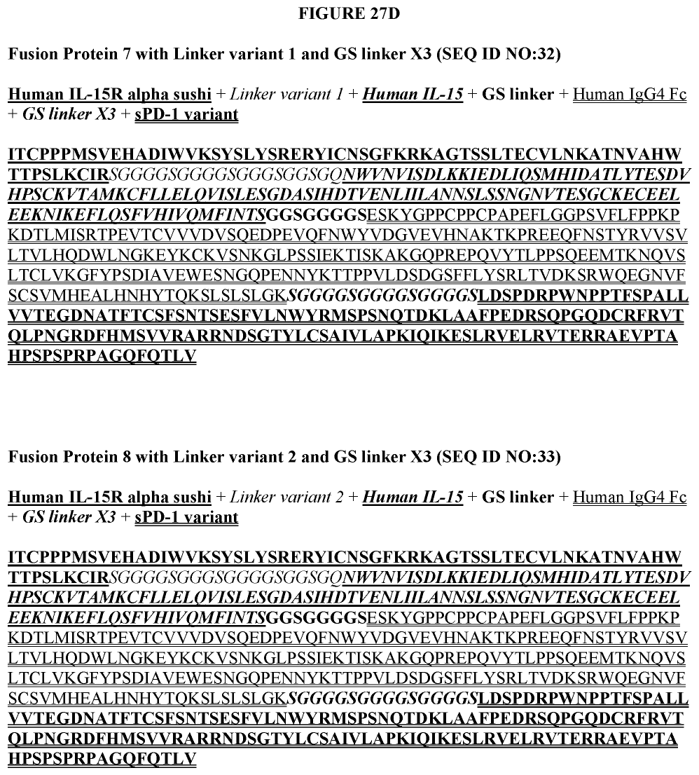

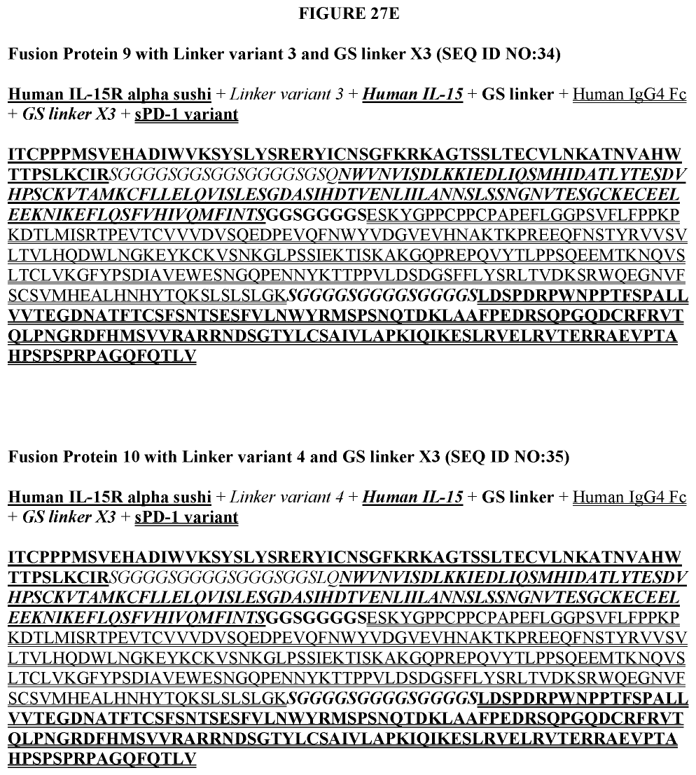

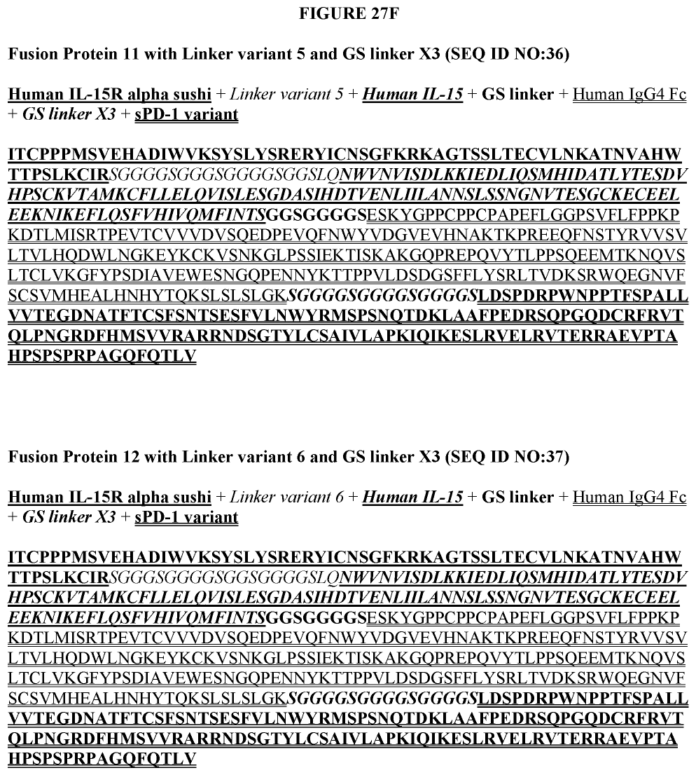

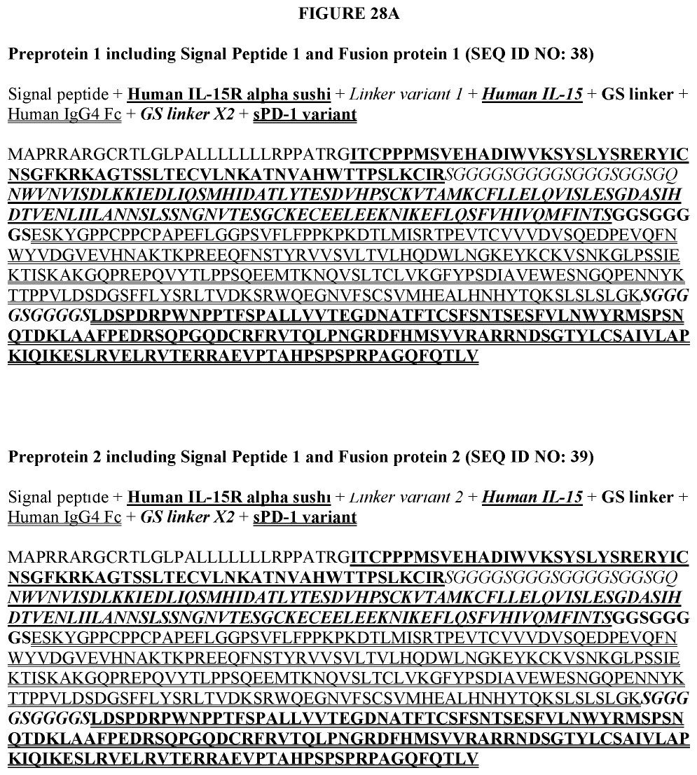

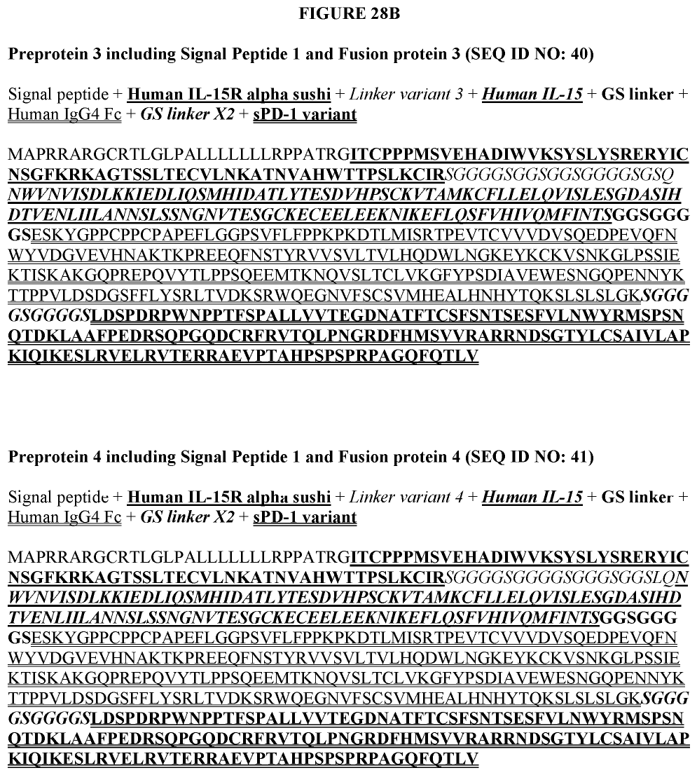

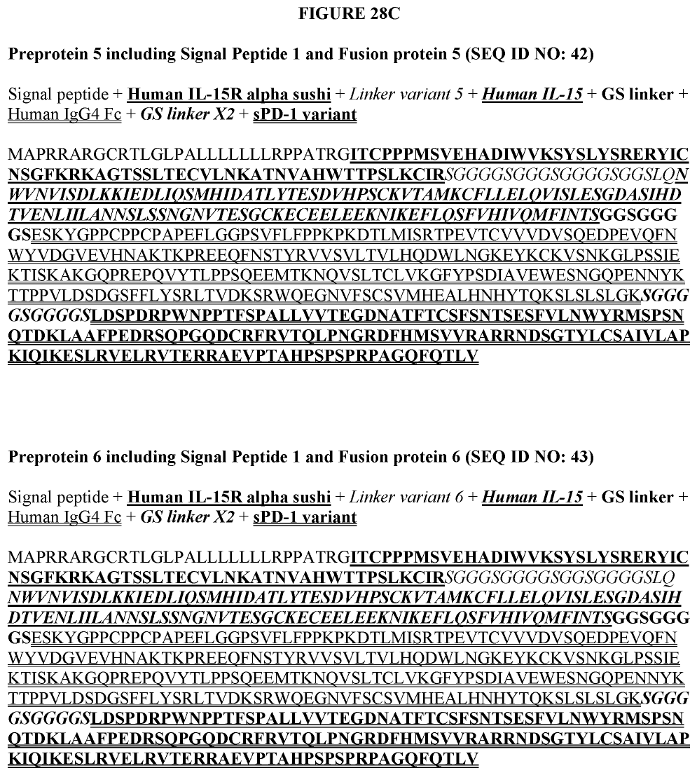

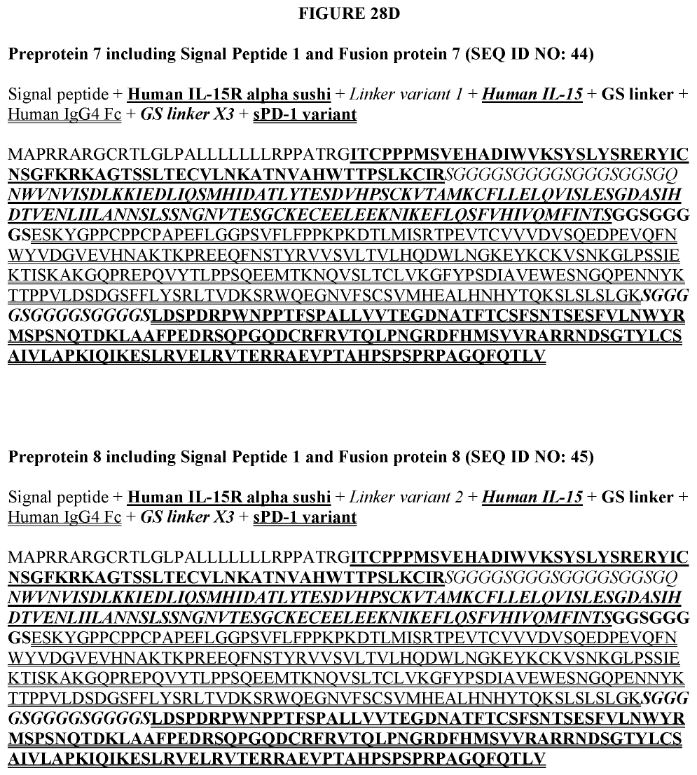

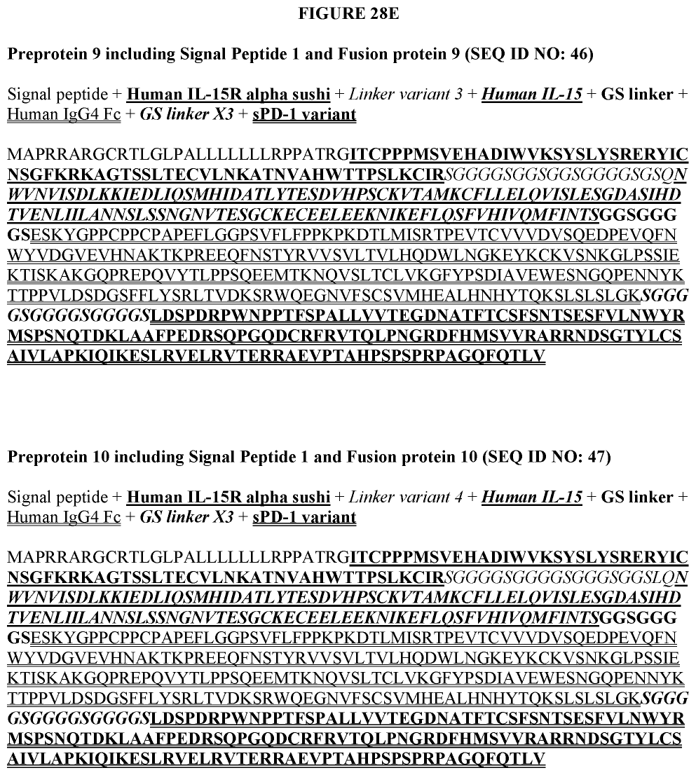

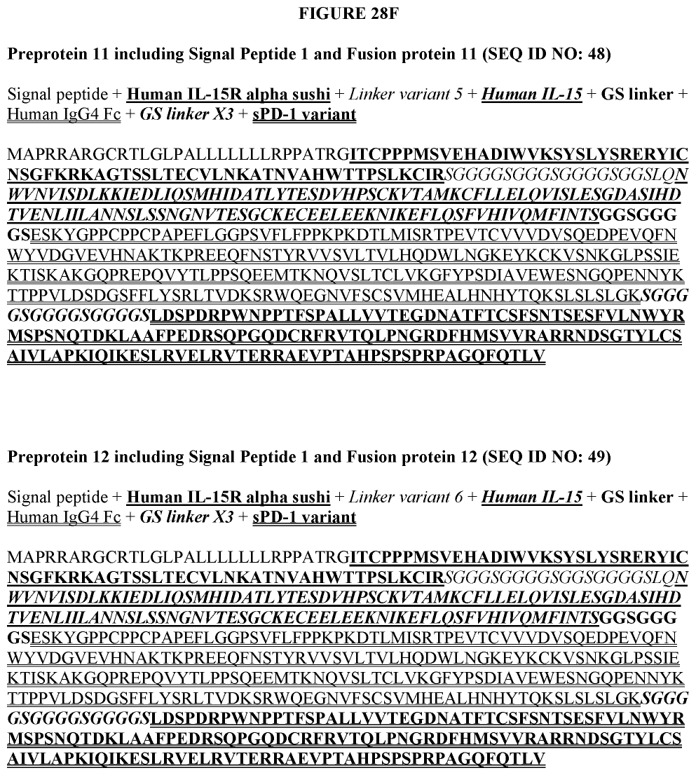

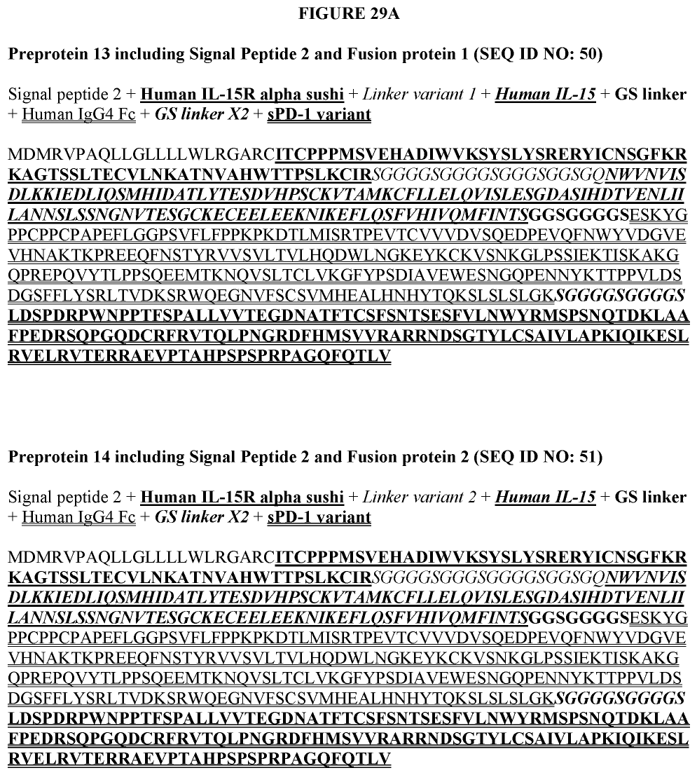

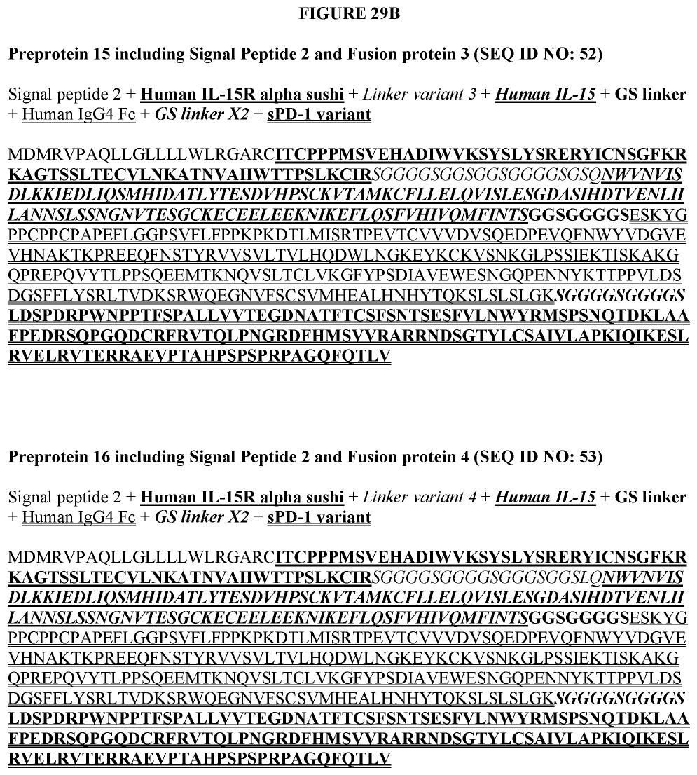

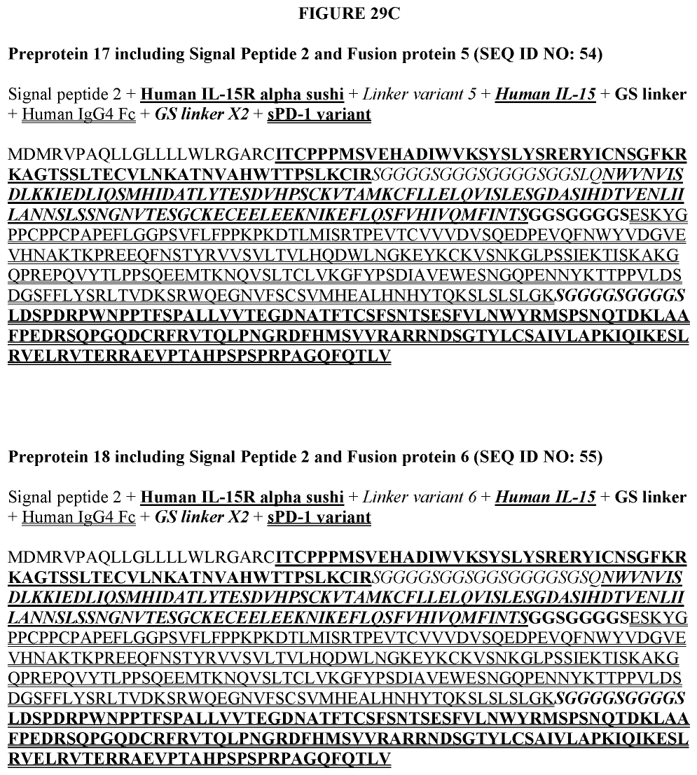

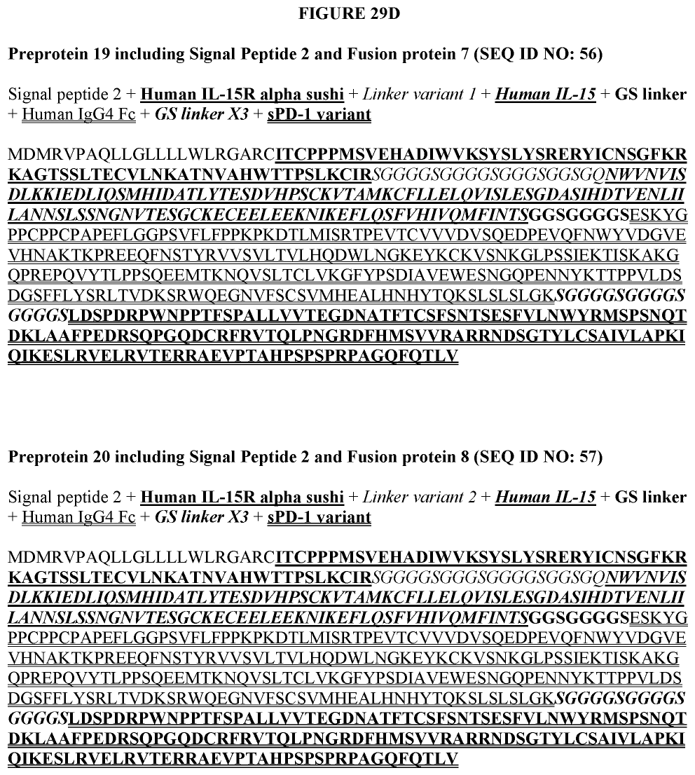

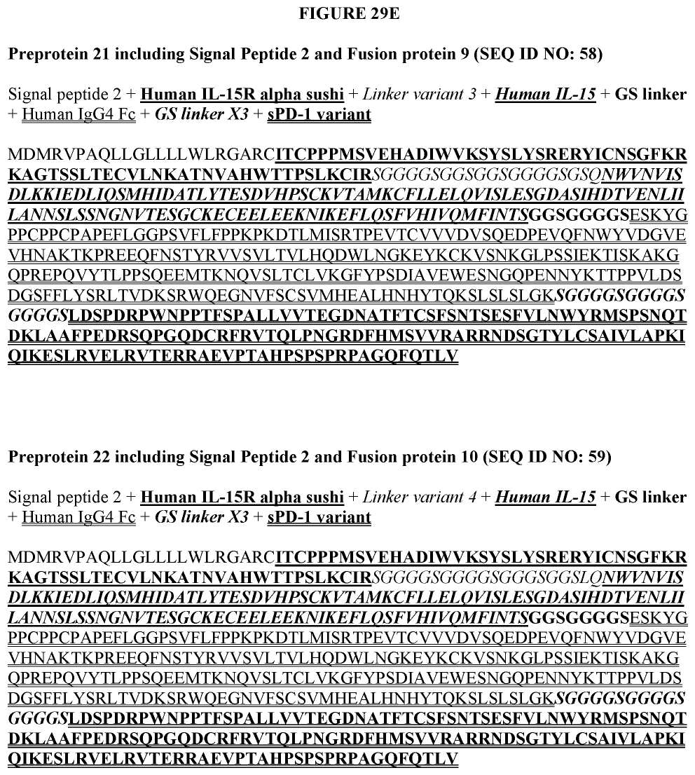

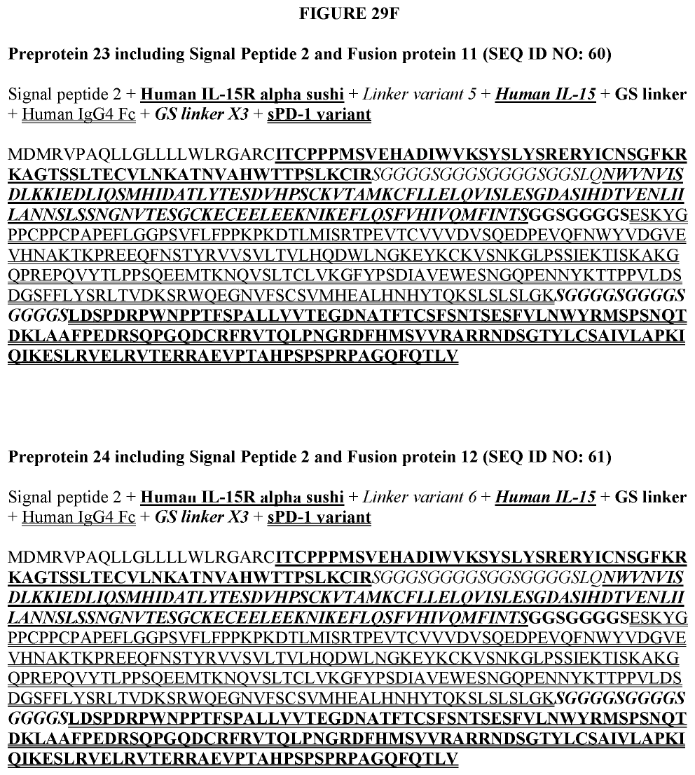

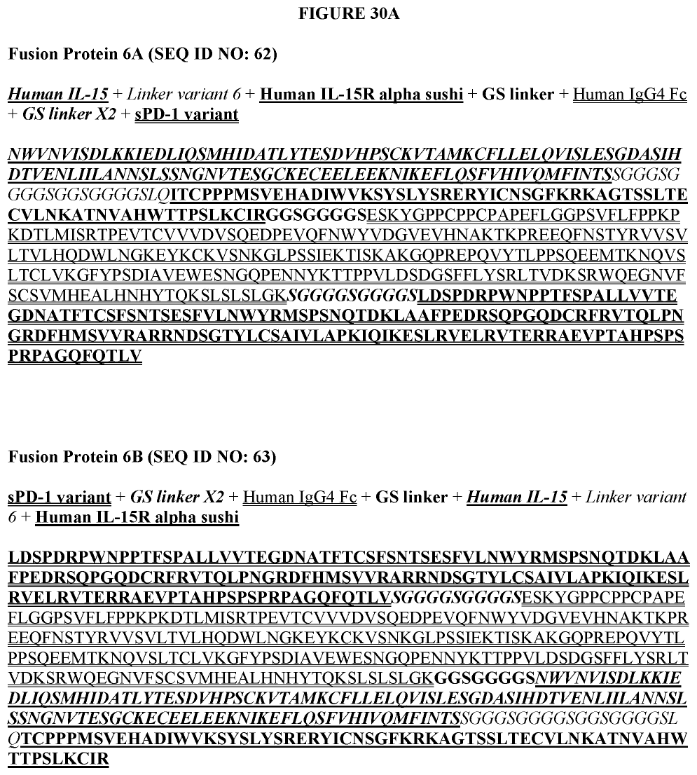

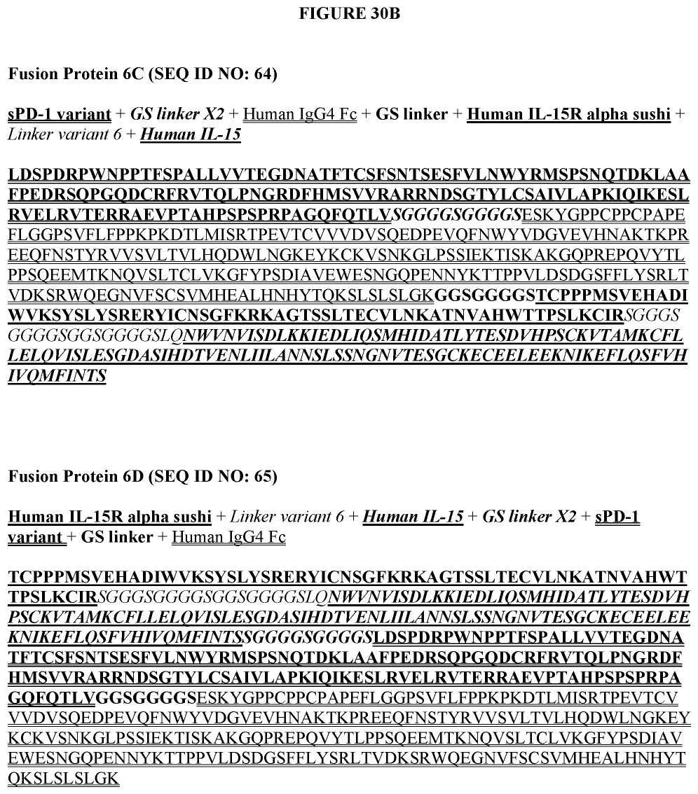

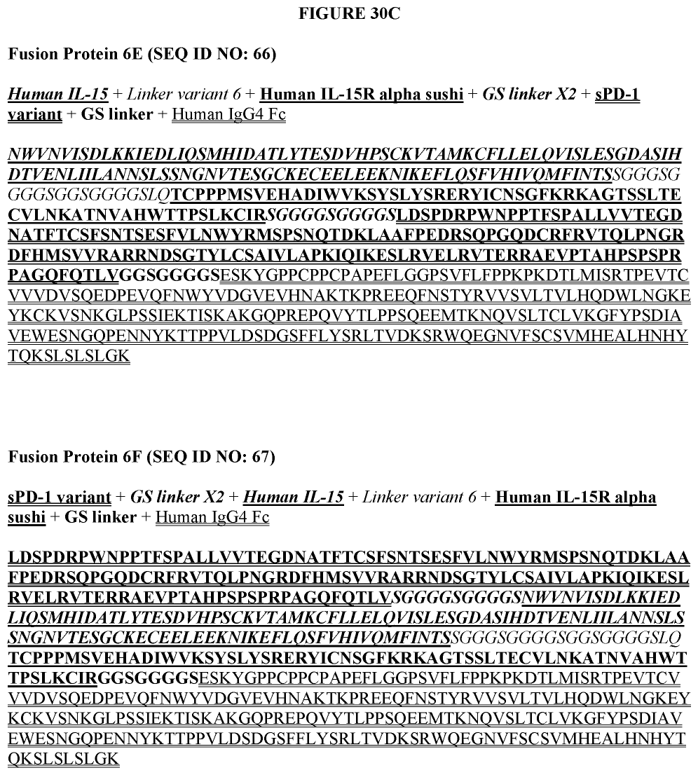

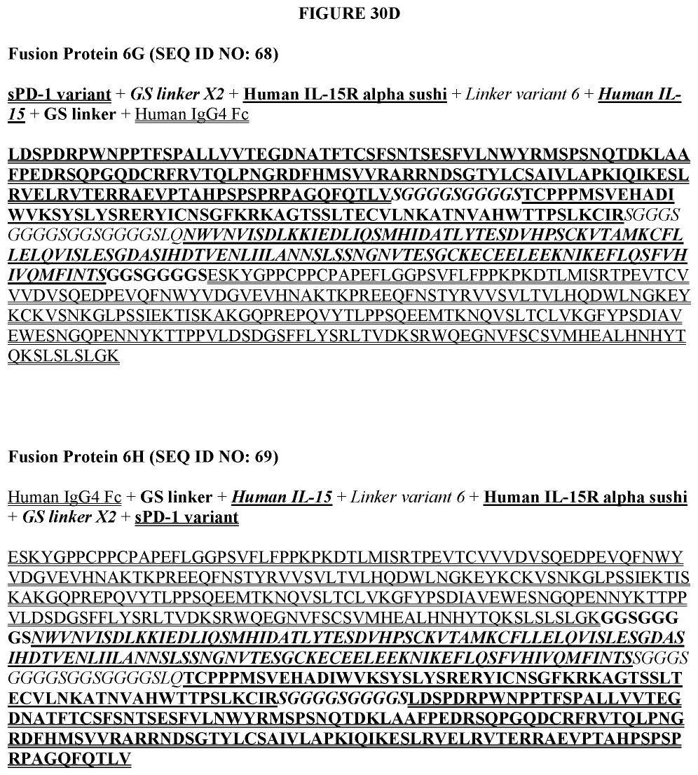

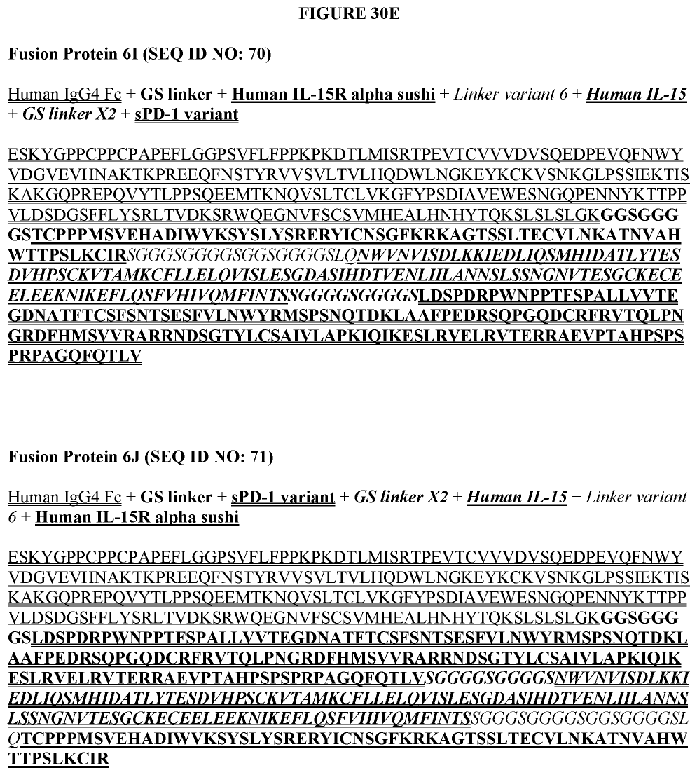

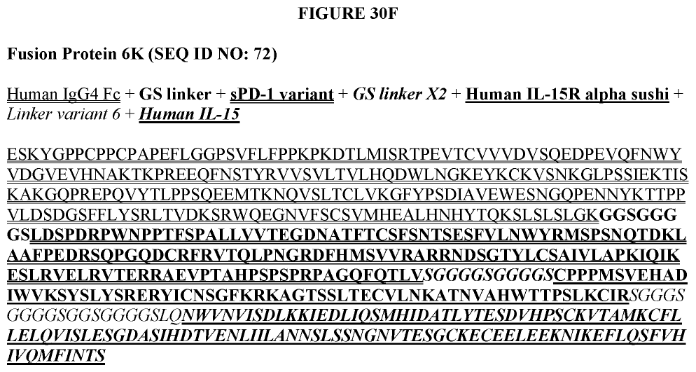

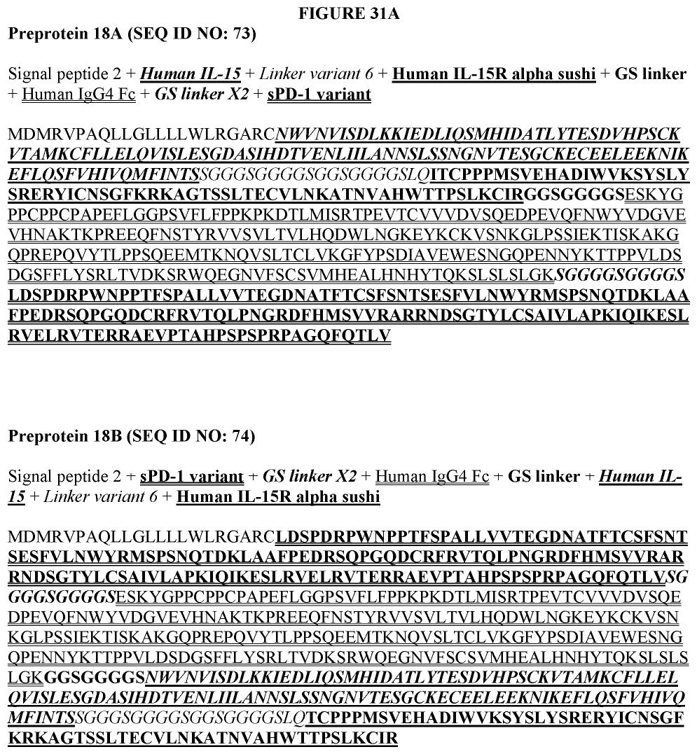

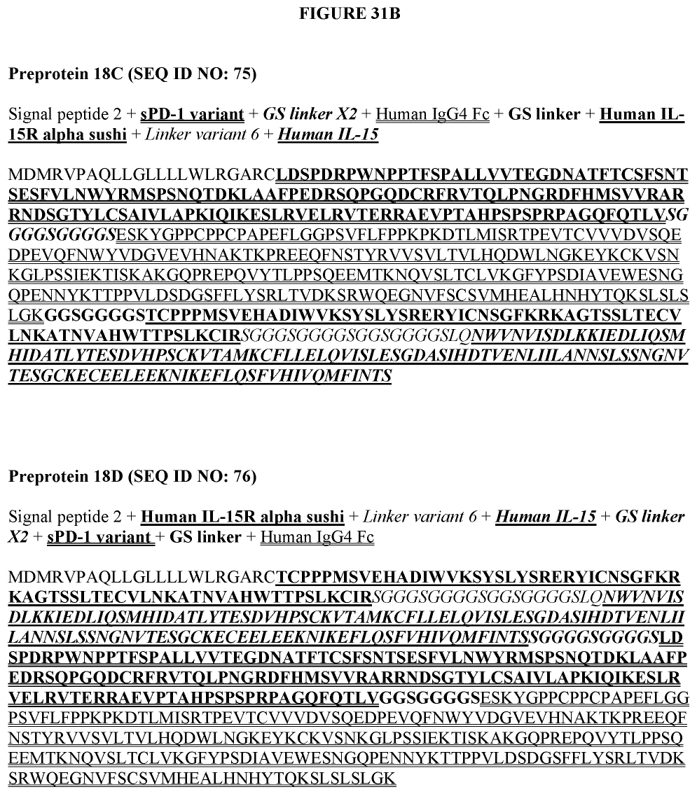

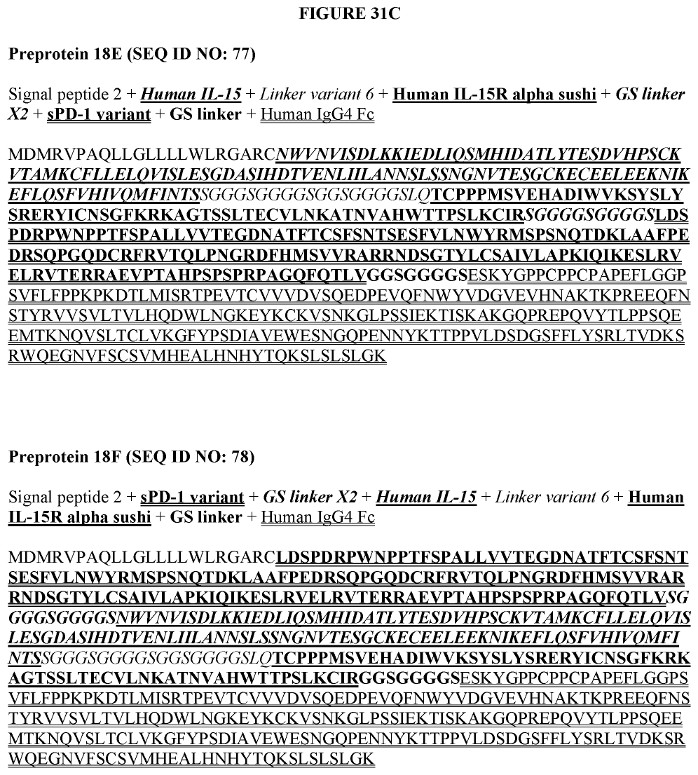

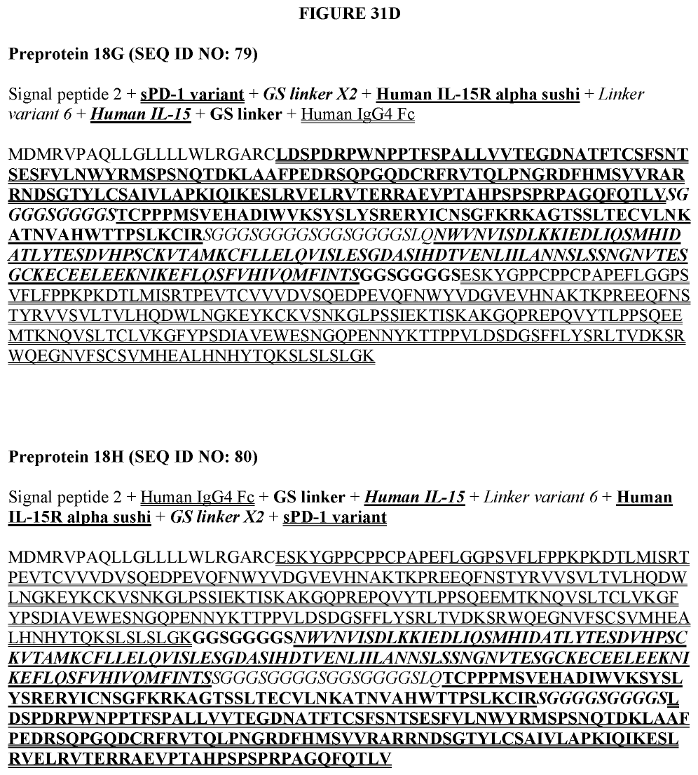

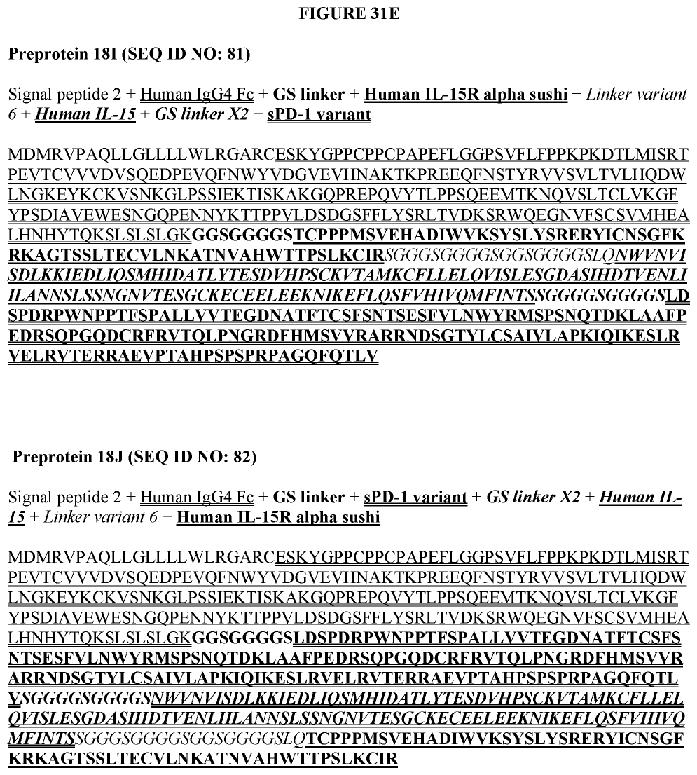

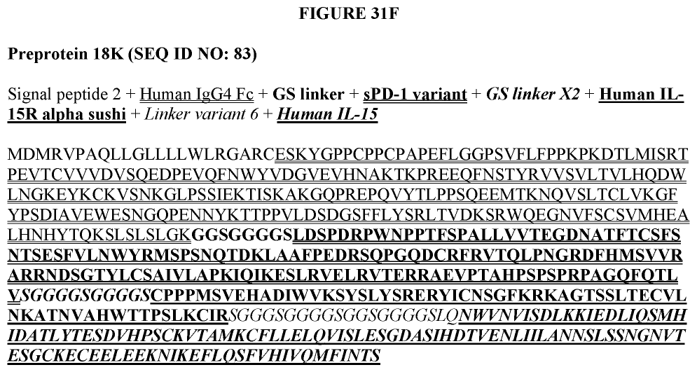

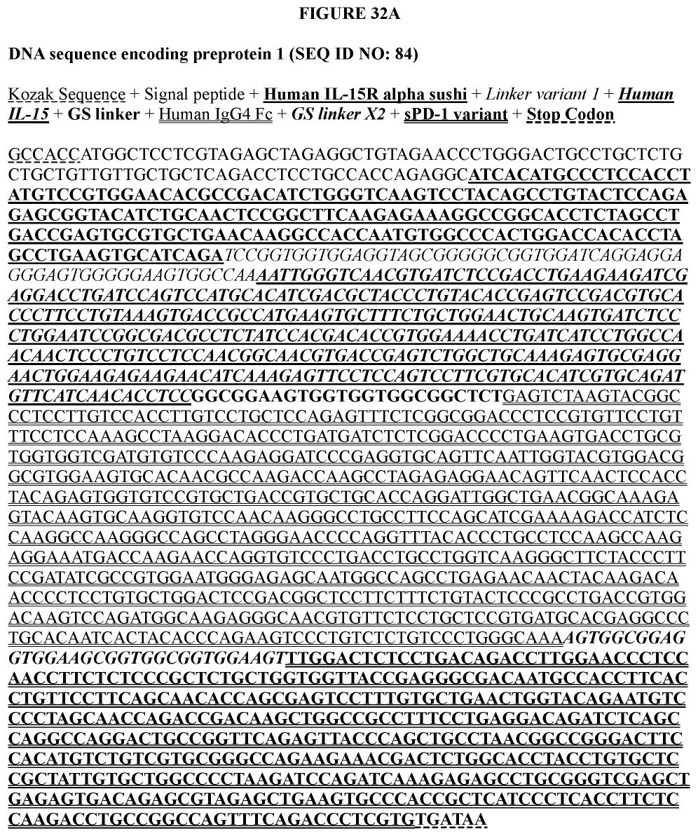

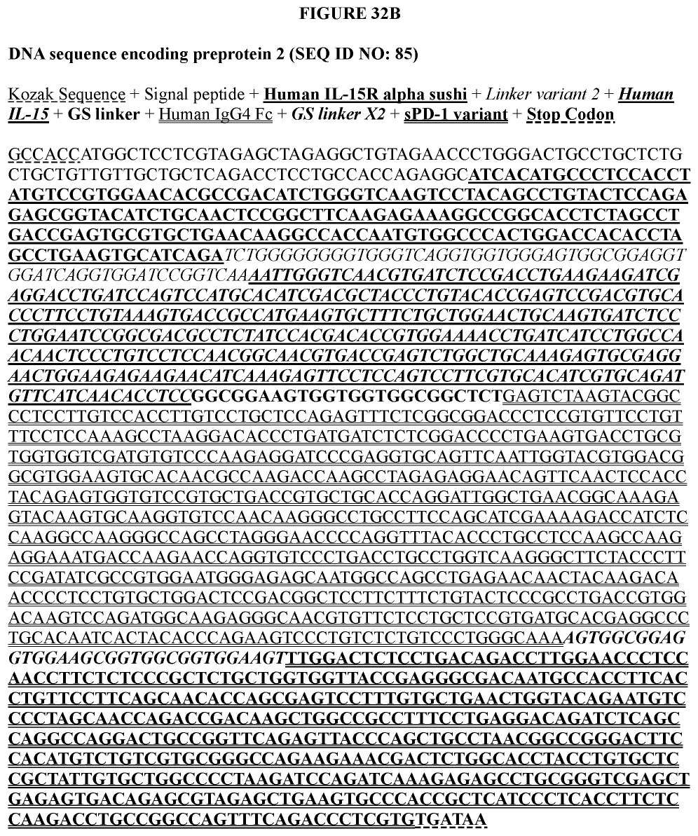

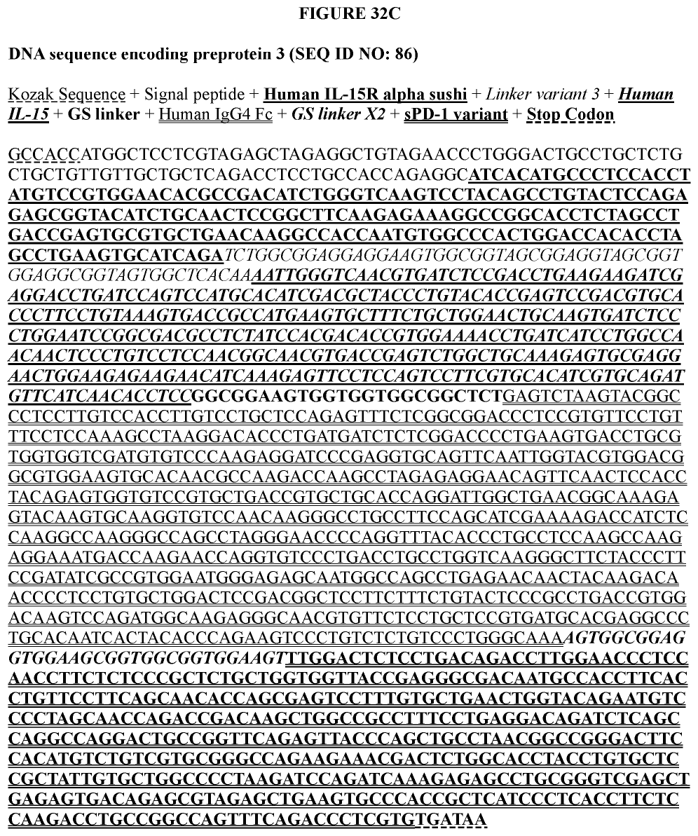

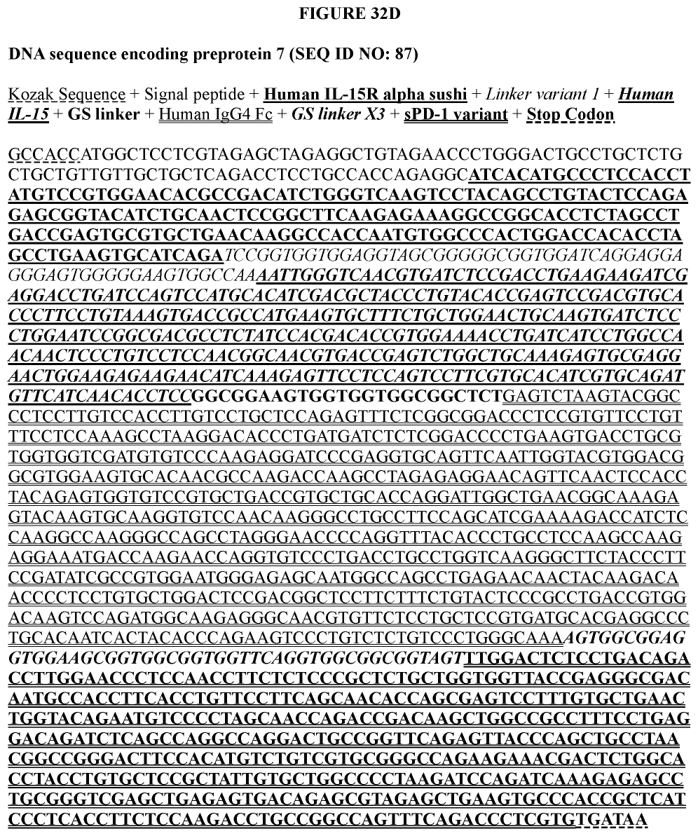

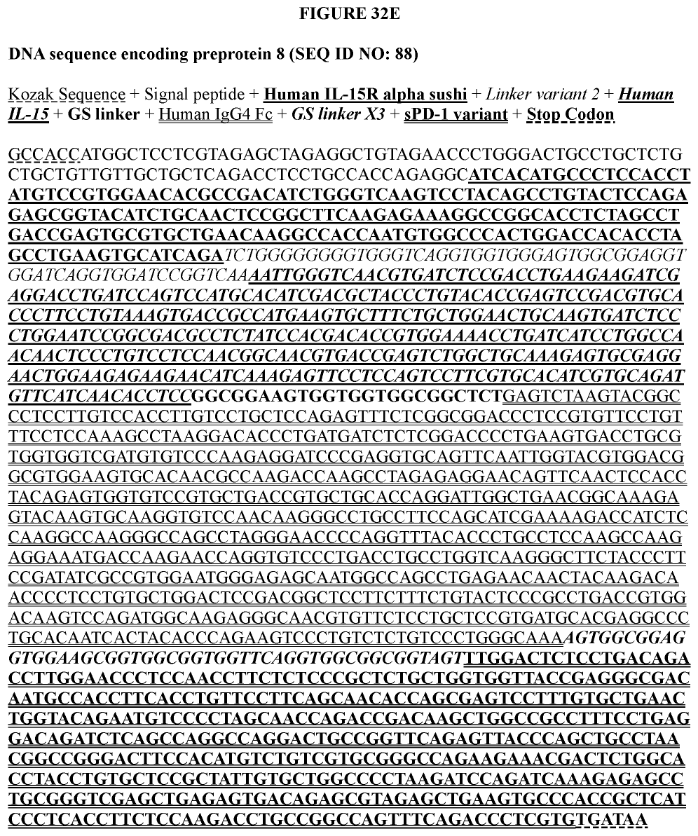

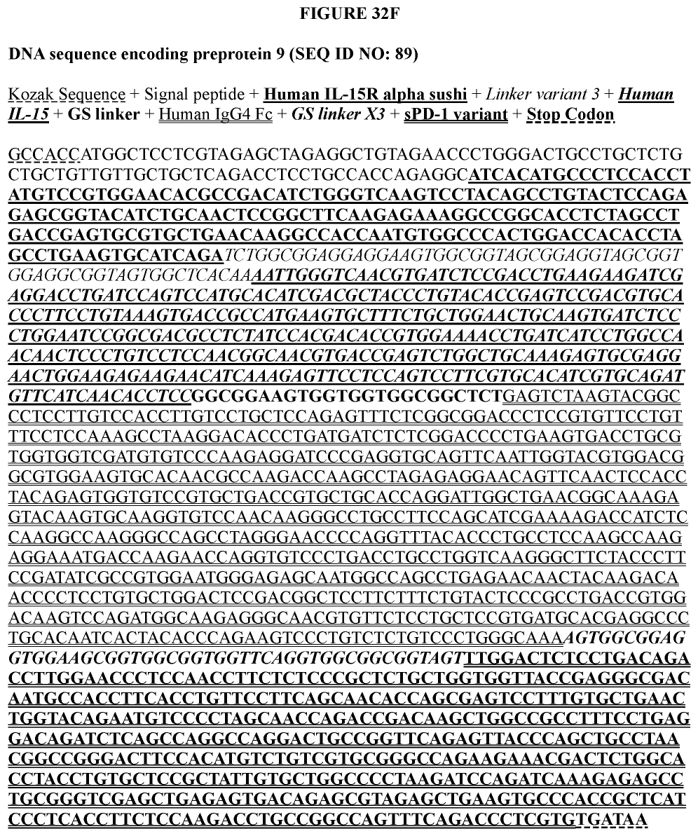

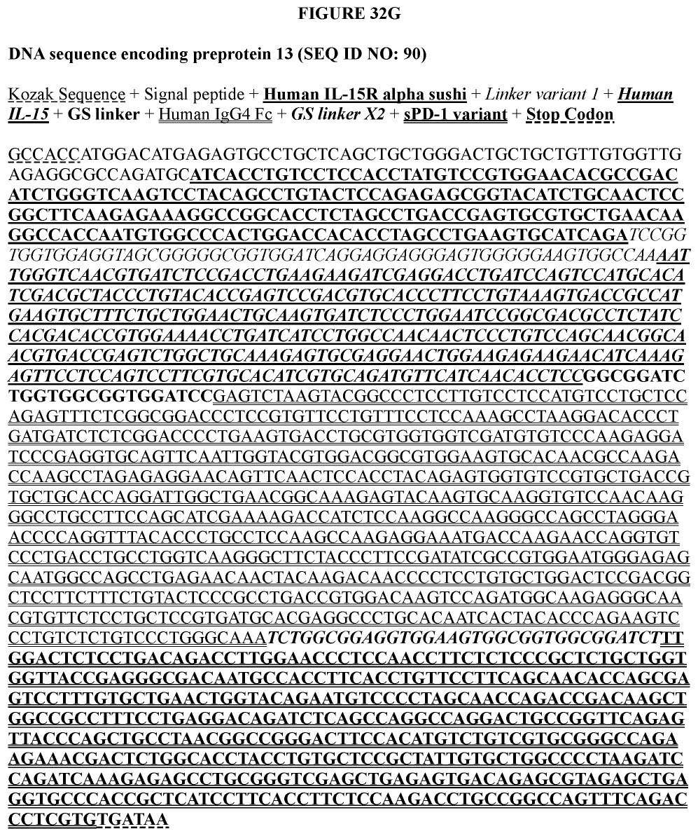

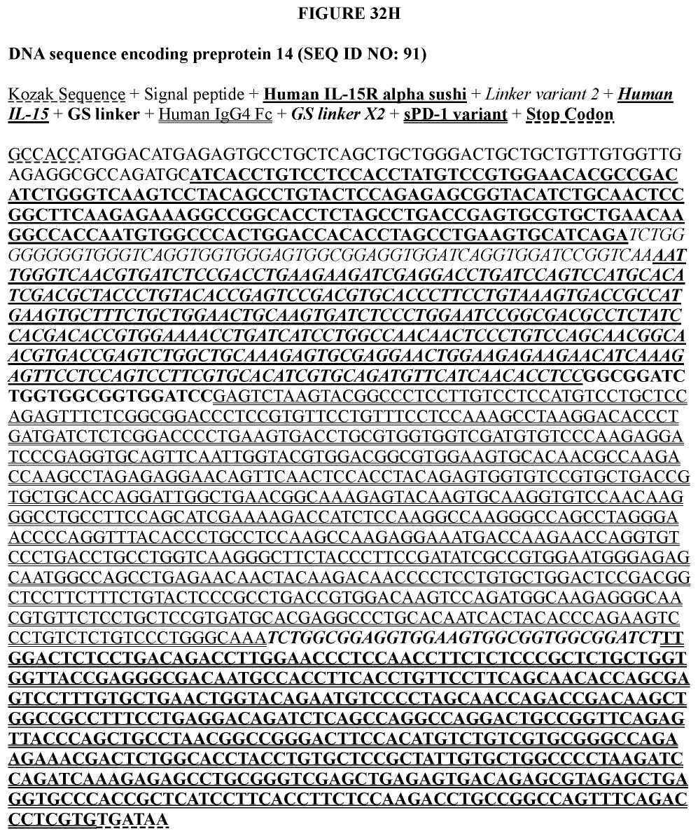

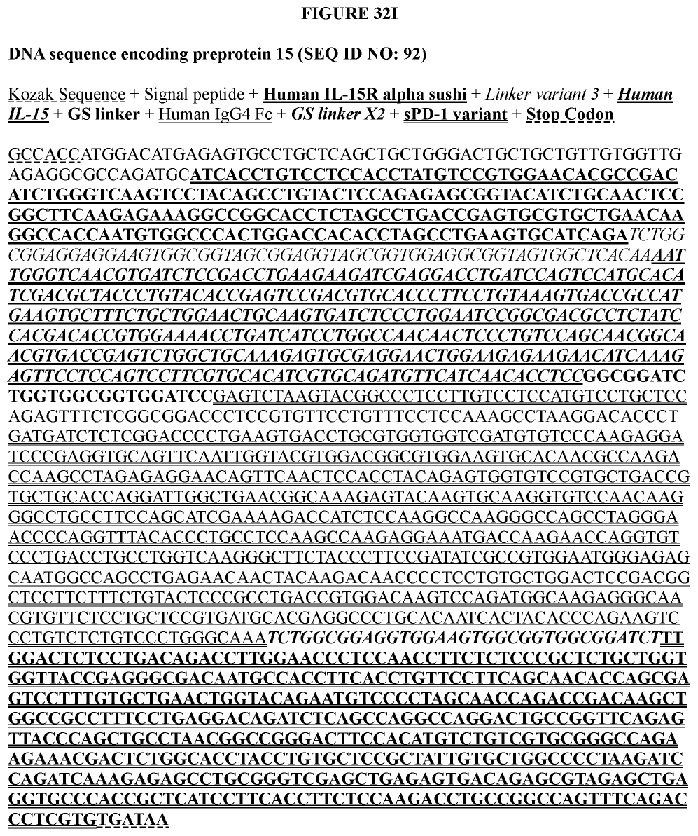

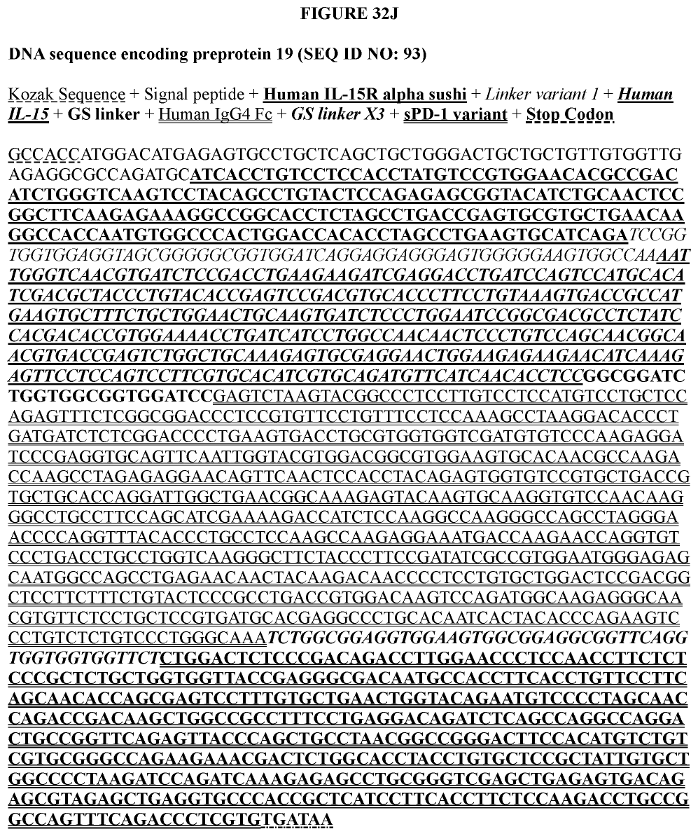

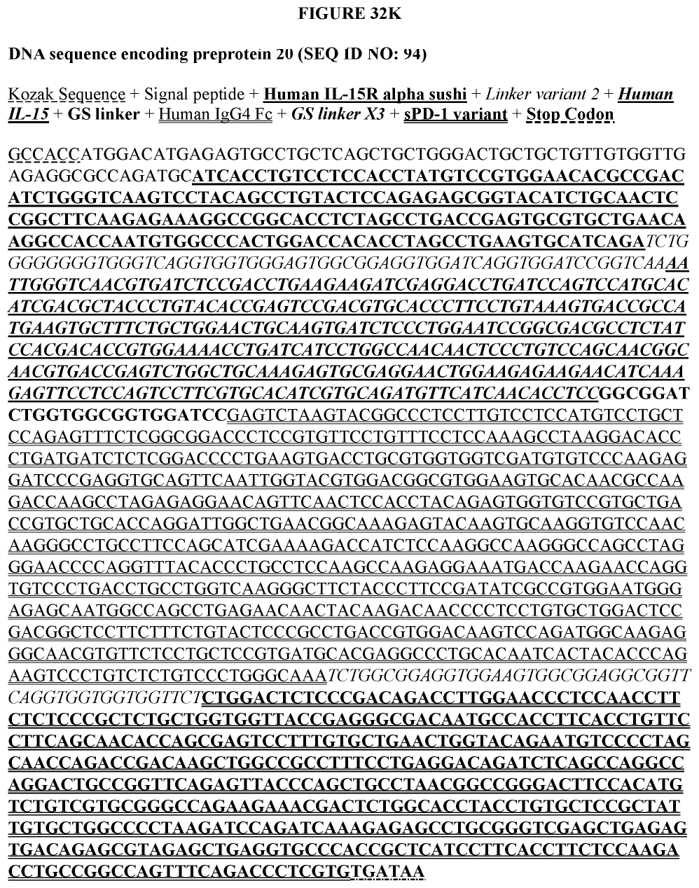

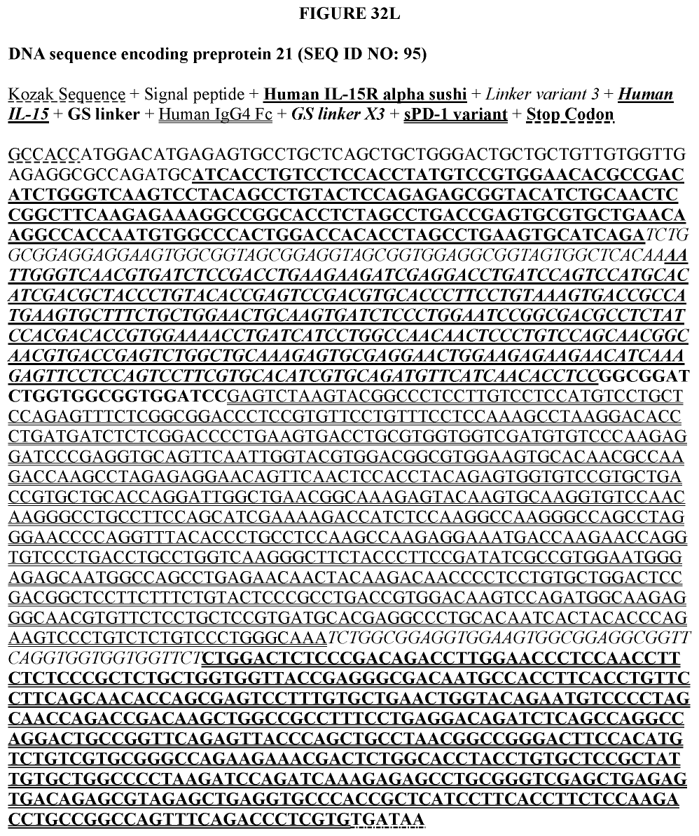

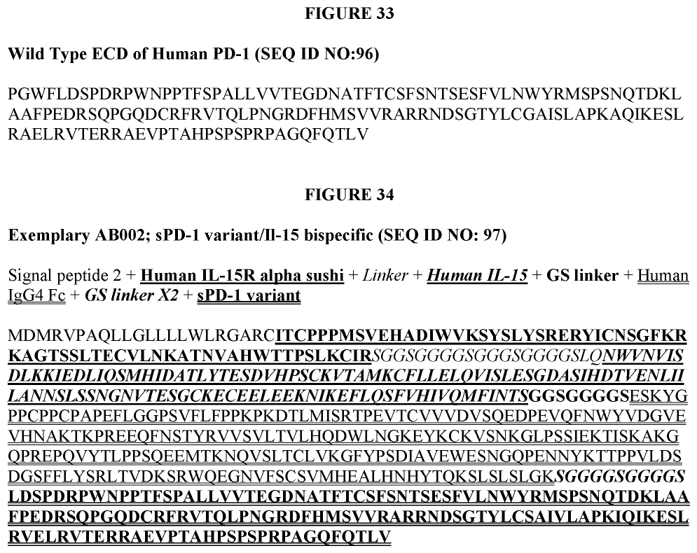

This study is designed to investigate: 1) whether changes in administrative route from intravenous (IV) dosing to subcutaneous (SQ) dosing alter the therapeutic efficacy of AB002; 2) whether the inclusion of sPD-1 domain in AB002 contributes towards enhanced antitumor activity observed with AB002; and 3) whether the therapeutic efficacy of AB002 can be further enhanced when compared with PD-1 inhibitors in the form of sPD-1, aPD-1 Ab (pembrolizumab) or aPD-L1 Ab (atezolizumab). The tumor growth curve and final tumor volume of mice MC38 tumors treated with AB002 through IV or SQ and, the tumor growth curve and final tumor volume of MC38 tumors treated with sPD-1, calculated based on the molecular weight ratio equivalent to corresponding AB002 dosing and given as a monotherapy, are shown in are shown in FIG. 23 item A and FIG. 23 item B. The tumor growth curve and final tumor volume of MC38 tumors treated with AB002 in combination with sPD-1, aPD-1 Ab (pembrolizumab) or AB002+aPD-L1 Ab (atezolizumab) shown in FIG. 23 item C and FIG. 23 item D.

### Conclusion

1) Both intravenous and subcutaneous dosing of AB002 exhibits significant antitumor activity at a various concentration. 2) sPD-1 dosed at the molecular weight sratio equivalent to AB002 dosing concentration of 2.5 mg/kg or higher showed significant signal agent activity, suggesting it contributes to the antitumor efficacy of AB002 in an additive manner. 3) Addition of sPD-1, aPD-1 Ab (pembrolizumab) or AB002+aPD-L1 Ab (atezolizumab) did not further enhance the therapeutic efficacy of AB002, suggesting that AB002 treatment alone is sufficient to achieve optimal antitumor activity without combination with other immune checkpoint inhibitors.

1. A bispecific Fc fusion protein comprising:
a) an IL-15Rα sushi domain;
b) an IL-15 domain;
c) an Fc domain; and
d) a soluble PD-1 (sPD-1) variant domain.

2. The Fc fusion protein according to claim 1, further comprising:
a first domain linker, a second domain linker, and a third domain linker.

3. The Fc fusion protein according to claim 2, wherein the first domain linker, the second domain linker, and the third domain linker are selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, (GS)n, (GSGGS)n, (GGGGS)n, and (GGGS)n, wherein n is selected from the group consisting of 1, 2, 3, 4 and 5.

4. The Fc fusion protein according to claim 2, wherein the first domain linker is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

5. The Fc fusion protein according to claim 2, wherein the second domain linker is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

6. The Fc fusion protein according to claim 2, wherein the third domain linker is selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

7. The Fc fusion protein according to any preceding claim comprising, from N- to C-terminus:
a) the IL-15Rα sushi domain;
b) the first domain linker;
c) the IL-15 domain;
d) the second domain linker;
e) the Fc domain;
f) the third domain linker; and
g) the sPD-1 variant domain.

8. The Fc fusion protein according to claim 2 comprising, from N- to C-terminus:
a) the IL-15 domain;
b) the first domain linker;
c) the IL-15Rα sushi domain;
d) the second domain linker;
e) the Fc domain;
f) the third domain linker; and
g) the sPD-1 variant domain.

9. The Fc fusion protein according to claim 2 comprising, from N- to C-terminus:
a) the sPD-1 variant domain;
b) the first domain linker;
c) the Fc domain;
d) the second domain linker;
e) the IL-15 domain;
f) the third domain linker; and
g) the IL-15Rα sushi domain.

10. The Fc fusion protein according to claim 2 comprising, from N- to C-terminus:
a) the sPD-1 variant domain;
b) the first domain linker;
c) the Fc domain;
d) the second domain linker;
e) the IL-15Rα sushi domain;
f) the third domain linker; and
g) the IL-15 domain.

11. The Fc fusion protein according to claim 2 comprising, from N- to C-terminus:
a) the IL-15Rα sushi domain;
b) the first domain linker;
c) the IL-15 domain;
d) the second domain linker;
e) the sPD-1 variant domain;
f) the third domain linker; and
g) the Fc domain.

12. The Fc fusion protein according to claim 2 comprising, from N- to C-terminus:
a) the IL-15 domain;
b) the first domain linker;
c) the IL-15Rα sushi domain;
d) the second domain linker;
e) the sPD-1 variant domain;
f) the third domain linker; and
g) the Fc domain.

13. The Fc fusion protein according to claim 2 comprising, from N- to C-terminus:
a) the sPD-1 variant domain;
b) the first domain linker;
c) the IL-15 domain;
d) the second domain linker;
e) the IL-15Rα sushi domain;
f) the third domain linker; and
g) the Fc domain.

14. The Fc fusion protein according to claim 2 comprising, from N- to C-terminus:
a) the sPD-1 variant domain;
b) the first domain linker;
c) the IL-15Rα sushi domain;
d) the second domain linker;
e) the IL-15 domain;
f) the third domain linker; and
g) the Fc domain.

15. The Fc fusion protein according to claim 2 comprising, from N- to C-terminus:
a) the Fc domain;
b) the first domain linker;
c) the IL-15 domain;
d) the second domain linker;
e) the IL-15Rα sushi domain;
f) the third domain linker; and
g) the sPD-1 variant domain.

16. The Fc fusion protein according to claim 2 comprising, from N- to C-terminus:
a) the Fc domain;
b) the first domain linker;
c) the IL-15Rα sushi domain;
d) the second domain linker;
e) the IL-15 domain;
f) the third domain linker; and
g) the sPD-1 variant domain.

17. The Fc fusion protein according to claim 2 comprising, from N- to C-terminus:
a) the Fc domain;
b) the first domain linker;
c) the sPD-1 variant domain;
d) the second domain linker;
e) the IL-15 domain;
f) the third domain linker; and
g) the IL-15Rα sushi domain.

18. The Fc fusion protein according to claim 2 comprising, from N- to C-terminus:
a) the Fc domain;
b) the first domain linker;
c) the sPD-1 variant domain;
d) the second domain linker;
e) the IL-15Rα sushi domain;
f) the third domain linker; and
g) the IL-15 domain.

19. The Fc fusion protein according to claim 7,
wherein the first domain linker is selected from the group consisting of SEQ ID NO:13, SEQ ID NO:14, SEQ ID NO:15, SEQ ID NO:16, SEQ ID NO:17, and SEQ ID NO:18;
wherein the second domain linker is selected from the group consisting of SEQ ID NO:19, SEQ ID NO:20, and SEQ ID NO:21; and
wherein the third domain linker is selected from the group consisting of SEQ ID NO:19, SEQ ID NO:20, and SEQ ID NO:21.

20. The Fc fusion protein according to claim 9,
wherein the first domain linker is selected from the group consisting of SEQ ID NO:19, SEQ ID NO:20, and SEQ ID NO:21;
wherein the second domain linker is selected from the group consisting of SEQ ID NO:19, SEQ ID NO:20, and SEQ ID NO:21; and
wherein the third domain linker is selected from the group consisting of SEQ ID NO:13, SEQ ID NO:14, SEQ ID NO:15, SEQ ID NO:16, SEQ ID NO:17, and SEQ ID NO:18.

21-101. (canceled)
